(19) United States
(12) Patent Application Publication
(10) Pub. No.: US 2024/0148722 A1
(43) Pub. Date: May 9, 2024

(54) SMALL MOLECULES THAT BIND TO TDP-43 FOR THE TREATMENT OF ALS AND RELATED DISORDERS

(71) Applicant: Biohaven Therapeutics Ltd., New Haven, CT (US)

(72) Inventors: Randall J. BINDER, Philadelphia, PA (US); Marianne CARLSEN, Yardley, PA (US); Xiao JIN, King of Prussia, PA (US); Mark E. MCDONNELL, Lansdale, PA (US); Allen B. REITZ; Camille REMEUR, Toulouse (FR); Eric STROBEL, Warrington, PA (US); Huji TURDI, Yardley, PA (US)

(21) Appl. No.: 18/546,285

(22) PCT Filed: Feb. 20, 2022
(86) PCT No.: PCT/US2022/017116

§ 371 Date: Aug. 14, 2023

Related U.S. Application Data
(60) Provisional application No. 63/151,651, filed on Feb. 20, 2021.

(51) Int. Cl. A61K 31/496; A61K 31/415; A61K 31/428; A61K 31/443; A61K 31/444; A61K 31/454; A61K 31/5377; A61K 38/06; C07D 213/74; C07D 401/04; C07D 401/10; C07D 401/12; C07D 401/14; C07D 413/04; C07D 413/14; C07D 417/04; C07D 451/04; C07D 487/04; C07D 495/04

(52) U.S. Cl. CPC A61K 31/496; A61K 31/415; A61K 31/428; A61K 31/443; A61K 31/444; A61K 31/454; A61K 31/5377; A61K 38/06; C07D 213/74; C07D 401/04; C07D 401/10; C07D 401/12; C07D 401/14; C07D 413/04; C07D 413/14; C07D 417/04; C07D 451/04; C07D 487/04; C07D 495/04

(57) ABSTRACT

The present invention relates to compounds that bind to TDP-43 and may have a therapeutic effect in treating a human disease, such as amyotrophic lateral sclerosis, frontotemporal lobar degeneration, hippocampal sclerosis of aging, chronic traumatic encephalopathy, inclusion body myositis, Alzheimer's disease, and/or Alzheimer's disease related disorders.

### CROSS-REFERENCE TO RELATED APPLICATION

This application claims priority to U.S. Provisional Patent Application No. 63/151,651 filed on Feb. 20, 2021, and all the benefits accruing therefrom under 35 U.S.C. § 119, the content of which is incorporated herein in its entirety by reference.

### GOVERNMENT SUPPORT CLAUSE

This invention was made with government support under R44 AG059278-01 rights in the invention.

### FIELD OF THE INVENTION

The present invention relates to the use of compounds that bind to the protein trans-activating response (TAR) DNA binding protein TDP-43 and block the binding of nucleic acid to TDP-43. Such compounds may be useful for the treatment of amyotrophic lateral sclerosis (ALS), frontotemporal lobar degeneration of the TDP-43 type (FTLD-TDP-43), chronic traumatic encephalopathy (CTE) and/or inclusion body myositis (IBM).

### BACKGROUND OF THE INVENTION

ALS, FTLD-TDP-43, CTE and IBM are debilitating neurodegenerative disorders linked to TDP-43 and are considered as TDP-43 proteinopathies. ALS is an orphan disease (1:1000 lifetime risk, -35,000 patients US) characterized by diminished motor function resulting in muscle wasting and death typically 3-5 years from diagnosis, often due to respiratory failure. The primary genetic correlation comprising ˜40% of familial ALS patients involves the repeat (GGGGCC)_{600-1,000} in Orf92 on chromosome 9. A smaller subset (˜10%) of ALS patients have mutations in the earlier-discovered protein superoxide dismutase SOD1, with A4V mSOD1 being the most prevalent in North America (˜50% of SOD1 mutations). FTLD is now recognized to be the leading cause in cognitive decline, making it the second largest cause of dementia after Alzheimer's disease and accounting for 20% of all dementia. The extent and devastation of both ALS and FTLD are amplified by a broad impact upon caregivers and society with especially high costs in the terminal stages of the diseases. Unfortunately, there are no FDA-approved treatments for FTLD, and current trials are focusing on symptomatic treatment for cognitive decline or behavioral issues rather than disease-modifying therapies. One of the two currently FDA-approved treatments for ALS is riluzole, which increases lifespan by two to three months after 1.5 years of treatment. Although much research and applied drug discovery and development efforts have gone into understanding or modifying various pathways hypothesized to be important in ALS and FTLD disease pathogenesis, there remains unmet medical need for disease-modifying therapeutic relief that will slow or halt disease progression, improve quality of life, and extend lifespan.

### SUMMARY OF THE INVENTION

The present invention is directed to the modulation of TDP-43 in such a way as to provide therapeutic relief to patients suffering from conditions in which TDP-43 is implicated in the pathology of the disease.

In an embodiment, disclosed is a method for treating or preventing a disease that involves TDP-43, the method including administering to a subject an effective amount of at least one compound having formula (I):

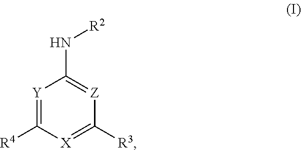

an enantiomer, a diastereomer, a hydrate, a solvate, a pharmaceutically acceptable salt, an isotopic analog, a prodrug or a complex thereof,

wherein, in formula (I):

X, Y, and Z are each independently N or CH;

R² is a substituted or unsubstituted C₁-C₂₀ linear, branched, or cyclic organic group including at least one nitrogen; and

R³ and R⁴ are each independently hydrogen, halogen, a hydroxyl group, a cyano group, a nitro group, a substituted or unsubstituted C₀-C₁₀ amino group, a substituted or unsubstituted C₁-C₁₀ alkyl group, a substituted or unsubstituted C₂-C₁₀ alkenyl group, a substituted or unsubstituted C₂-C₁₀ alkynyl group, and a substituted or unsubstituted C₁-C₁₀ alkoxy group, a substituted or unsubstituted C₃-C₁₀ cycloalkyl group, a substituted or unsubstituted C₂-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₂₀ aryl group, a substituted or unsubstituted C₆-C₂₀ aryloxy group, a substituted or unsubstituted C₆-C₂₀ arylthio group, a substituted or unsubstituted C₂-C₂₀ heteroaryl group, a substituted or unsubstituted C₂-C₂₀ heteroaryloxy group, or a substituted or unsubstituted C₂-C₂₀ heteroarylthio group.

In another embodiment, disclosed is a method for treating or preventing a disease that involves TDP-43, the method including administering to a subject an effective amount of at least one compound having formula (II):

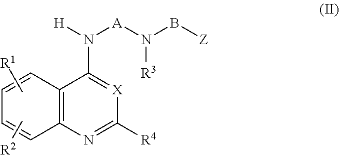

an enantiomer, a diastereomer, a hydrate, a solvate, a pharmaceutically acceptable salt, an isotopic analog, a prodrug or a complex thereof,

wherein, in formula (II):

X is N or CH;

R¹ and R² are the same or different and are each independently H, halogen, C₁-C₄ alkyl, C₁-C₄ alkoxy, CF₃, or CF₃O, wherein C₁-C₄ alkyl and C₁-C₄ alkoxy are optionally substituted with at least one fluorine;

R³ is H, C₁-C₇ alkyl, C₄-C₇ carbocyclic, aryl, heteroaryl, heterocyclic, heterocyclic alkyl, wherein R³ is optionally taken together with either A or B to form a ring consisting of 4-7 ring members;

R⁴=phenyl or phenyl substituted with C₁-C₄ alkyl, C₁-C₄ alkoxy, CF₃, CF₃O, halogen, amino, or sulfonamide;

A=(CH₂)ₙ, wherein n is 2-4, wherein A and R³ are optionally taken together to form a ring consisting of 4-7 ring members;

B=(CH₂)ₙ, wherein n is 2-6, wherein B and R³ are optionally taken together to form a ring consisting of 4-7 members, and wherein B is optionally substituted with one or more substituents selected from C₁-C₄ alkyl, hydroxy, C₁-C₄ alkoxy, CF₃, and CF₃O; and

Z is OH, C₁-C₄ alkoxy, OCF₃, or N with one or more of H, C₁-C₄ alkyl, heteroaryl, substituted heteroaryl, C₁-C₄ sulfonamido, substituted amido.

In another embodiment, disclosed is a method for treating or preventing a disease that involves TDP-43, the method including administering to a subject an effective amount of at least one compound having formulae (IIIa) or (IIIb):

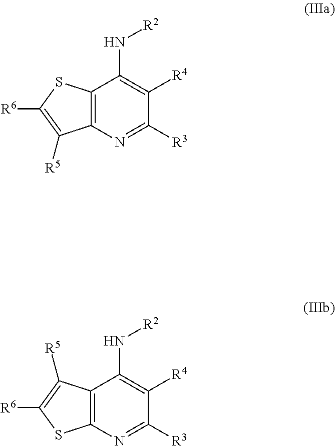

an enantiomer, a diastereomer, a hydrate, a solvate, a pharmaceutically acceptable salt, an isotopic analog, a prodrug or a complex thereof,

wherein, in formulae (IIIa) and (IIIb):

R² is a substituted or unsubstituted C₁-C₂₀ linear, branched, or cyclic organic group including at least one nitrogen; and

R³, R⁴, R⁵, and R⁶ are each independently hydrogen, halogen, a hydroxyl group, a cyano group, a nitro group, a substituted or unsubstituted C₀-C₁₀ amino group, a substituted or unsubstituted C₁-C₁₀ alkyl group, a substituted or unsubstituted C₂-C₁₀ alkenyl group, a substituted or unsubstituted C₂-C₁₀ alkynyl group, and a substituted or unsubstituted C₁-C₁₀ alkoxy group, a substituted or unsubstituted C₃-C₁₀ cycloalkyl group, a substituted or unsubstituted C₂-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₂₀ aryl group, a substituted or unsubstituted C₆-C₂₀ aryloxy group, a substituted or unsubstituted C₆-C₂₀ arylthio group, a substituted or unsubstituted C₂-C₂₀ heteroaryl group, a substituted or unsubstituted C₂-C₂₀ heteroaryloxy group, or a substituted or unsubstituted C₂-C₂₀ heteroarylthio group.

In another embodiment, disclosed is a method for treating or preventing a disease that involves TDP-43, the method including administering to a subject an effective amount of at least one compound having formula (IV):

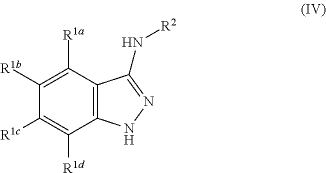

an enantiomer, a diastereomer, a hydrate, a solvate, a pharmaceutically acceptable salt, an isotopic analog, a prodrug or a complex thereof,

wherein, in formula (IV):

R² is a substituted or unsubstituted C₁-C₂₀ linear, branched, or cyclic organic group including at least one nitrogen; and

R^{1a}, R^{1b}, R^{1c}, and R^{1d} are each independently hydrogen, halogen, a hydroxyl group, a cyano group, a nitro group, a substituted or unsubstituted C₀-C₁₀ amino group, a substituted or unsubstituted C₁-C₁₀ alkyl group, a substituted or unsubstituted C2-C₁₀ alkenyl group, a substituted or unsubstituted C₂-C₁₀ alkynyl group, and a substituted or unsubstituted C₁-C₁₀ alkoxy group, a substituted or unsubstituted C₃-C₁₀ cycloalkyl group, a substituted or unsubstituted C2-C₁₀ heterocycloalkyl group, a substituted or unsubstituted C3-C₁₀ cycloalkenyl group, a substituted or unsubstituted C2-C₁₀ heterocycloalkenyl group, a substituted or unsubstituted C₆-C₂₀ aryl group, a substituted or unsubstituted C₆-C₂₀ aryloxy group, a substituted or unsubstituted C₆-C₂₀ arylthio group, a substituted or unsubstituted C₂-C₂₀ heteroaryl group, a substituted or unsubstituted C₂-C₂₀ heteroaryloxy group, or a substituted or unsubstituted C₂-C₂₀ heteroarylthio group.

In another embodiment, disclosed is a method for treating or preventing a disease that involves TDP-43, the method including administering to a subject an effective amount of at least one compound having formula (V):

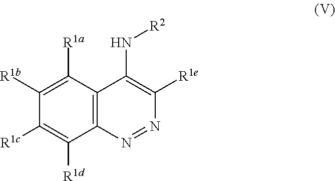

an enantiomer, a diastereomer, a hydrate, a solvate, a pharmaceutically acceptable salt, an isotopic analog, a prodrug or a complex thereof,

wherein, in formula (V):

R² is a substituted or unsubstituted C₁-C₂₀ linear, branched, or cyclic organic group including at least one nitrogen; and

R^{1a}, R^{1b}, R^{1c}, R^{1d}, and R^{1c} are each independently hydrogen, halogen, a hydroxyl group, a cyano group, a nitro group, a substituted or unsubstituted C₀-C₁₀ amino group, a substituted or unsubstituted C₁-C₁₀ alkyl group, a substituted or unsubstituted C₂-C₁₀ alkenyl group, a substituted or unsubstituted C₂-C₁₀ alkynyl group, and a substituted or unsubstituted C₁-C₁₀ alkoxy group, a substituted or unsubstituted C₃-C₁₀ cycloalkyl group, a substituted or unsubstituted C₂-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₂₀ aryl group, a substituted or unsubstituted C₆-C₂₀ aryloxy group, a substituted or unsubstituted C₆-C₂₀ arylthio group, a substituted or unsubstituted C₂-C₂₀ heteroaryl group, a substituted or unsubstituted C₂-C₂₀ heteroaryloxy group, or a substituted or unsubstituted C₂-C₂₀ heteroarylthio group.

In another embodiment, disclosed is a method for treating or preventing a disease associated with TDP-43 proteinopathies, the method including administering to a subject an effective amount of at least one of the above compounds.

In another embodiment, disclosed is a method for treating or preventing diseases that involve excess amounts of TDP-43 in the cytosol, the method including administering to a subject an effective amount of at least one of the above compounds.

In another embodiment, disclosed is a method of use of the TDP-43 binders of the present invention as positron emission tomography (PET) imaging agents, wherein the method includes administering to a subject an effective amount of an isotopically labeled compound recited above.

In another embodiment, disclosed is a method of use of the TDP-43 binders of the present invention as single-photon emission computed tomography (SPECT) imaging agents, wherein the method includes administering to a subject an effective amount of an isotopically labeled compound recited above.

In an embodiment, the disease may be amyotrophic lateral sclerosis (ALS).

In another embodiment, the disease may be frontotemporal lobar degeneration (FTLD).

In another embodiment, the disease may be chronic traumatic encephalopathy (CTE).

In another embodiment, the disease may be hippocampal sclerosis of aging (CARTS).

In another embodiment, the disease may be inclusion body myositis (IBM).

In another embodiment, the disease may be Alzheimer's disease (AD).

In another embodiment, the disease may be Alzheimer's disease (AD) related disorders.

In another embodiment, the disease may be peripheral including myopathy.

In an embodiment, the small-molecule TDP-43 binders may be used in combination with other agents to treat the disease.

### BRIEF DESCRIPTION OF THE DRAWINGS

These and/or other aspects will become apparent and more readily appreciated from the following description of the embodiments, taken in conjunction with the accompanying drawings, in which:

FIG. 1: SH-SY5Y and A172 cells were seeded in 6 well plates at 1 ×106 and 2×105 cells/well respectively. Approximately 18 hours after seeding, MG132 and compounds were added at the indicated concentrations and incubation at 37° C. was continued for 20 hours. The cells were harvested and cytoplasmic fractions were prepared according to the supplier's protocol using the Thermo NE-PER kit (#78833). The samples were denatured in 0.1M DTT, Novex NuPAGE LDS Sample Buffer at 80° C. for 10 minutes, separated on 12% polyacrylamide Tris-Glycine SDS gels, transferred to PVDF membranes and probed with a C-terminal TDP-43-specific antibody (ProteinTech 12892-1-AP).

### DETAILED DESCRIPTION OF THE INVENTION

The following detailed description is provided to aid those skilled in the art in practicing the present invention. Those of ordinary skill in the art may make modifications and variations in the embodiments described herein without departing from the spirit or scope of the present disclosure. Unless otherwise defined, all technical and scientific terms used herein have the same meaning as commonly understood by one of ordinary skill in the art to which this disclosure belongs. The terminology used in the description is for describing particular embodiments only and is not intended to be limiting.

As used in this application, except as otherwise expressly provided herein, each of the following terms shall have the meaning set forth below. Additional definitions are set forth throughout the application. In instances where a term is not specifically defined herein, that term is given an art-recognized meaning by those of ordinary skill applying that term in context to its use in describing the present invention.

The TDP-43 binding agents may be present as isotopically labeled forms of compounds detailed herein. Isotopically labeled compounds have structures depicted by the formulas given herein except that one or more atoms are replaced by an atom having a selected atomic mass or mass number. Examples of isotopes that can be incorporated into compounds of the disclosure include isotopes of hydrogen, carbon, nitrogen, oxygen, phosphorous, fluorine and chlorine, such as, but not limited to ²H (deuterium, D), ³H (tritium), ¹³C, ¹⁴C, ¹⁵N, ¹⁸F, ³¹P, ³²p, ³⁵S, Cl and I.

Various isotopically labeled compounds of the present disclosure, for example those into which radioactive isotopes such as 3H, ¹³C and 14C are incorporated, are provided. Such isotopically labeled compounds may be useful in metabolic studies, reaction kinetic studies, detection or imaging techniques, such as positron emission tomography (PET) or single-photon emission computed tomography (SPECT) including drug or substrate tissue distribution assays or in radioactive treatment of subjects (e.g. humans). Also provided for isotopically labeled compounds described herein are any pharmaceutically acceptable salts, or hydrates, as the case may be.

In some variations, the compounds disclosed herein may be varied such that from 1 to “n” hydrogens attached to a carbon atom is/are replaced by deuterium, in which “n” is the number of hydrogens in the molecule. Such compounds may exhibit increased resistance to metabolism and are thus useful for increasing the half-life of the compound when administered to a subject. See, for example, Foster, “Deuterium Isotope Effects in Studies of Drug Metabolism”, Trends Pharmacol. Sci. 5(12):524-527 (1984). Such compounds are synthesized by means well known in the art, for example by employing starting materials in which one or more hydrogens have been replaced by deuterium.

Deuterium labeled or substituted therapeutic compounds of the disclosure may have improved drug metabolism and pharmacokinetics (DMPK) properties, relating to absorption, distribution, metabolism and excretion (ADME). Substitution with heavier isotopes such as deuterium may afford certain therapeutic advantages resulting from greater metabolic stability, for example increased in vivo half-life, reduced dosage requirements and/or an improvement in therapeutic index. An ¹⁸F labeled compound may be useful for PET or SPECT studies.

Isotopically labeled compounds of this disclosure can generally be prepared by carrying out the procedures known to those skilled in the art by substituting a readily available isotopically labeled reagent for a non-isotopically labeled reagent. It is understood that deuterium in this context is regarded as a substituent in the compounds provided herein.

The concentration of such a heavier isotope, specifically deuterium, may be defined by an isotopic enrichment factor. In the compounds of this disclosure any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition.

The pharmaceutical compositions of the present invention including a TDP-43 binding agent typically also include other pharmaceutically acceptable carriers and/or excipients such as binders, lubricants, diluents, coatings, disintegrants, barrier layer components, glidants, coloring agents, solubility enhancers, gelling agents, fillers, proteins, co-factors, emulsifiers, solubilizing agents, suspending agents and mixtures thereof. A skilled artisan in the art would know what other pharmaceutically acceptable carriers and/or excipients could be included in the formulations according to the invention. The choice of excipients would depend on the characteristics of the compositions and on the nature of other pharmacologically active compounds in the formulation. Appropriate excipients are known to those skilled in the art (see Handbook of Pharmaceutical Excipients, fifth edition, 2005 edited by Rowe et al., McGraw Hill) and have been utilized to yield a novel sublingual formulation with unexpected properties.

Examples of pharmaceutically acceptable carriers that may be used in preparing the pharmaceutical compositions of the present invention may include, but are not limited to, fillers such as sugars, including lactose, sucrose, mannitol, or sorbitol; cellulose preparations such as maize starch, wheat starch, rice starch, potato starch, gelatin, gum tragacanth, methyl cellulose, hydroxypropyl methyl-cellulose, sodium carboxymethylcellulose, polyvinyl-pyrrolidone (PVP), talc, calcium sulphate, vegetable oils, synthetic oils, polyols, alginic acid, phosphate buffered solutions, emulsifiers, isotonic saline, pyrogen-free water and combinations thereof. If desired, disintegrating agents may be combined as well, and exemplary disintegrating agents may be, but not limited to, cross-linked polyvinyl pyrrolidone, agar, or alginic acid or a salt thereof such as sodium alginate. The compositions may be prepared by any of the methods of pharmacy but all methods include the step of bringing into association one or more chemical agents as described above with the carrier which constitutes one or more necessary ingredients. In general, the pharmaceutical compositions of the present invention may be manufactured in conventional methods known in the art, for example, by means of conventional mixing, dissolving, granulating, dragee-making, levigating, emulsifying, encapsulating, entrapping, lyophilizing processes and the like.

Identifying the subject in need of such treatment can be in the judgment of the subject or a health care professional and can be subjective (e.g., opinion) or objective (e.g., measurable by a test or diagnostic method). The identified subject may be an animal or human in need thereof, particularly a human. Such treatment will be suitably administered to subjects, particularly humans, suffering from the disease.

Typical dosage frequencies for TDP-43 modulating agents include once a day, twice a day, three times a day, four times a day, once every other day, once a week, twice a week, three times a week, four times a week, once every two weeks, once or twice monthly, and the like.

Compounds of the present invention are prepared by several synthetic pathways readily applied by those skilled in the art of synthetic chemistry. In the first method (Scheme 1 and 2), a 2,4,6-trichloropyridine, pyrimidine, triazine or benzene is subjected to a Suzuki coupling reaction, consisting of reaction with an aryl boronic acid in the presence of PddppfCl₂, TBAB and Na₂CO₃ in Dioxane water (2:1). The resulting 2-aryl pyridine (Intermediate A), pyrimidine, triazine or benzene is then reacted with an aryl or alkyl boronic acid in the presence of PddppfCl₂, TBAB and NaCO₃ in Dioxane water (2:1). The disubstituted pyridine, pyrimidine, triazine or benzene (Intermediate C, E) Scheme 2, is reacted with a primary, secondary amine, using standard Buchwald coupling reaction, consisting of reacting with a primary, secondary amine in the presence of Pd₂dba₃, X-Phos, NaOtBu in t-Butanol. Further homologation of intermediates to final products can be achieved as needed by standard condensation, reductive amination, deprotection and coupling reactions. Further reaction Intermediate D with an aldehyde under standard reductive amination reaction conditions known to those skilled in the art would produce further homologated compounds of the invention. In another embodiment Scheme 3 the 4-aryl-pyridine (Intermediate B) is then reacted with an aryl or alkyl boronic acid in the presence of PddppfCl₂, TBAB and Na₂CO₃ in Dioxane water (2:1). The 2,4—disubstituted pyridine is reacted with a primary amine, using standard Buchwald coupling reaction, consisting of reacting with a primary amine in the presence of Pd₂dba₃, X-Phos, NaOtBu in t-Butanol.

Final products are purified by normal phase silica gel chromatography using solvent mixtures containing dichloromethane, methanol and/or ammonium hydroxide, or preparative HPLC chromatography using solvent mixtures such as high purity water and acetonitrile modified with trifluoroacetic acid and characterized by proton NMR spectroscopy and mass spectrometry.

### Embodiments

In an aspect, the present invention is directed toward novel small molecules capable of binding to TDP-43 (TDP-43 binders), a compound of formula (I),

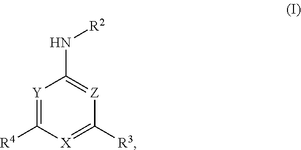

an enantiomer, a diastereomer, a hydrate, a solvate, a pharmaceutically acceptable salt, an isotopic analog, a prodrug or a complex thereof, wherein:

wherein, in formula (I):

X, Y, and Z are each independently N or CH;

R² is a substituted or unsubstituted C₁-C₂₀ linear, branched, or cyclic organic group including at least one nitrogen; and

R³ and R⁴ are each independently hydrogen, halogen, a hydroxyl group, a cyano group, a nitro group, a substituted or unsubstituted C₀-C₁₀ amino group, a substituted or unsubstituted C₁-C₁₀ alkyl group, a substituted or unsubstituted C₂-C₁₀ alkenyl group, a substituted or unsubstituted C₂-C₁₀ alkynyl group, and a substituted or unsubstituted C₁-C₁₀ alkoxy group, a substituted or unsubstituted C₃-C₁₀ cycloalkyl group, a substituted or unsubstituted C₂-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₂₀ aryl group, a substituted or unsubstituted C₆-C₂₀ aryloxy group, a substituted or unsubstituted C₆-C₂₀ arylthio group, a substituted or unsubstituted C₂-C₂₀ heteroaryl group, a substituted or unsubstituted C₂-C₂₀ heteroaryloxy group, or a substituted or unsubstituted C₂-C₂₀ heteroarylthio group.

The present invention is further directed toward novel small molecules capable of binding to TDP-43 (TDP-43 binders), a compound of formula (I),

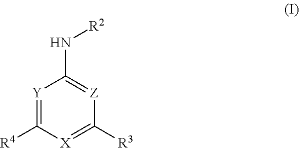

an enantiomer, a diastereomer, a hydrate, a solvate, a pharmaceutically acceptable salt, an isotopic analog, a prodrug or a complex thereof, wherein:

X, Y, and Z are each independently N or CH;

R² is selected from the group consisting of —(CH₂)ₙ—NR⁵R⁶, —(CH₂),C(O)—NR⁵R⁶, —(CH₂)ₙO(CH₂)ₙ—NR⁵R⁶, —(CH₂)ₙCF₂(CH₂)ₙ

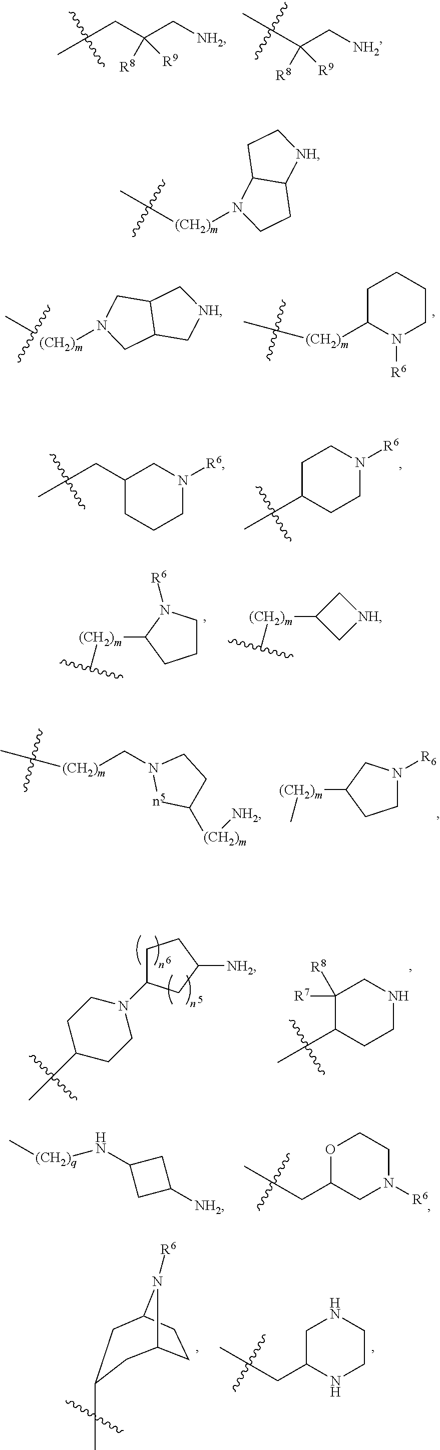

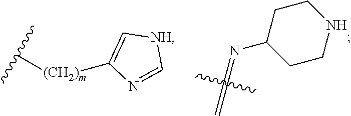

n⁵is 1 or 2;

n⁶ is 1 or 2;

q is 1 or 2;

m is 1 or 2;

R⁵ is selected from the group consisting of H, CH₃, (CH₂),NH₂, C(O)O(CH₂)m;

R⁶ is selected from the group consisting of H, CH₃, (CH₂),NH₂, C(O)O(CH₂)m;

R⁷ is at each occurrence independently selected from the group consisting of H and F;

R⁸ is at each occurrence independently selected from the group consisting of H and F;

R³ is hydrogen; CF₃; a five-membered monocyclic heteroaryl ring including at least one heteroatom selected from the group consisting from O, N, and S that is optionally substituted with up to 2 groups selected from C₁₋₄ linear alkyl, C₃₋₄ branched alkyl, C₁₋₄ linear alkoxy, C₃₋₄ branched alkoxy, CF₃, CF₃O, halogen

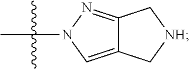 [SMILES: *n1cc2c(n1)CNC2]

a five-membered monocyclic heteroaryl ring including at least one heteroatom selected from the group consisting from O, N, and S; a phenyl ring that is optionally substituted with up to 2 groups selected from C₁₋₄ linear alkyl, C₃₋₄ branched alkyl, C₁₋₄ linear alkoxy, C₃₋₄ branched alkoxy, C₃₋₆ cycloalkyl, C₃0.6 cycloalkoxy, CF₃, CF₃O, halogen, NR^{3a}R^{3b}, SO₂NHR^{3a},

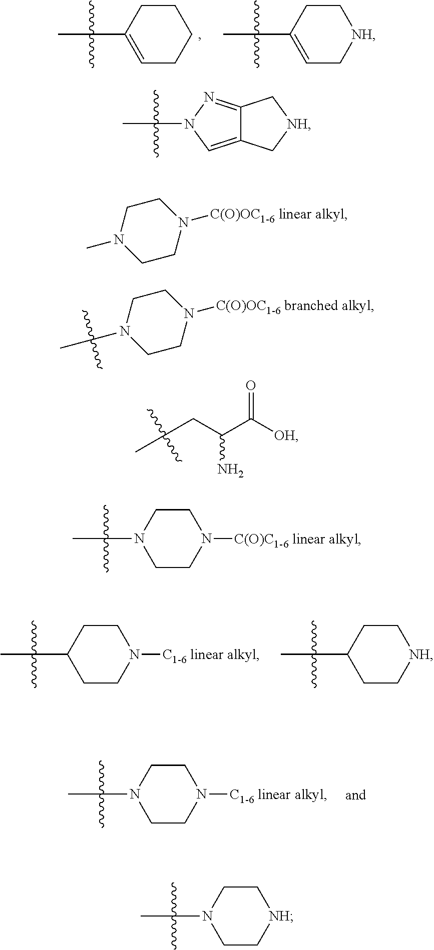

a pyridine ring that is optionally substituted with up to two groups selected from

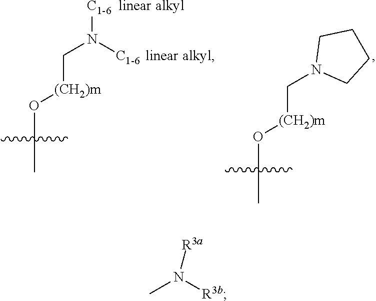

R^{3a} is at each occurrence independently selected from the group consisting of C₁₋₄ linear alkyl and C₃_₄ branched alkyl;

R^{3b} is at each occurrence independently selected from the group consisting of C₁₋₄ linear alkyl and C₃_₄ branched alkyl;

m is 1 or 2;

R⁴ is hydrogen; a phenyl ring optionally substituted with up to two groups selected from, F, linear alkoxy, branched alkoxy, CF₃O,

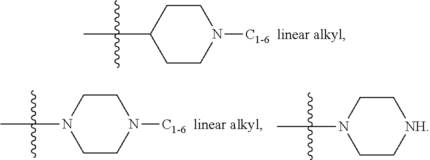

In an embodiment of Formula (I), X may be N, Y may be CH, and Z may be CH. In another embodiment, X may be CH, Y may be N, and Z may be CH. In another embodiment, X may be CH, Y may be CH, and Z may be N. In another embodiment, X may be N, Y may be N, and Z may be CH. In another embodiment, X may be CH, Y may be N, and Z may be N. In another embodiment, X may be N, Y may be CH, and Z may be N. In another embodiment, X, Y, and Z may each be N. In another embodiment, X, Y, and Z may each be CH.

In an embodiment of Formula (I), R² may be an amino(C₃-C₇)cycloalkyl group, for example, an aminocyclopropyl group, an aminocyclobutyl group, an aminocyclopentyl group, an aminocyclohexyl group, or aminocycloheptyl group. The amino group (—NH₂) may be attached to any position of the cycloalkyl group. For example, an aminocyclobutyl group may be 2-aminocyclobutyl group or 3-aminocyclobutyl group. In another example, an aminocyclopentyl group may be 2-aminocyclopentyl group or 3-aminocyclopentyl group. In yet another example, an aminocyclohexyl group may be 2-aminocyclopentyl group, 3-aminocyclohexyl group or 4-aminocyclopentyl group. The configuration of the amino groups at the cycloalkyl ring may be cis- or trans-. For example, R² may be a cis-aminocyclopropyl group, a trans-aminocyclopropyl group, a cis-aminocyclobutyl group, a trans-aminocyclobutyl group, a cis-aminocyclopentyl group, a trans-aminocyclopentyl group, a cis-aminocyclohexyl group, a trans-aminocyclohexyl group, a cis-aminocycloheptyl group or a trans-aminocycloheptyl group. In another embodiment, R² may be a (C₁-C₅alkyl)₂amino(C₃-C₇)cycloalkyl group, wherein the “amino(C₃-C₇)cycloalkyl group” is the same as described above.

In an embodiment of Formula (I), R³ may be a substituted or unsubstituted phenyl group. The substituted phenyl group may be a para-substituted phenyl group, a meta-substituted phenyl group, an ortho-substituted phenyl group, or a combination thereof. For example, R³ may be a phenyl group substituted in a para-position with a group listed above as a non-limited example of the “substituted” term. For example, R³ may be a phenyl group (C₆H5—), a chlorophenyl group (ClC₆H₄—), or a methoxyphenyl group (CH₃OC₆H₄—).

In an embodiment of Formula (I), R⁴ may be a (C₀-C₅alkyl)₂amino(C₂-C₅)alkyloxy group. For example, R⁴ may be a (C₁-C₅alkyl)₂amino(C₂-C₅)alkyloxy group, such as a (C₁-C₅alkyl)₂aminoethyloxy group, a (C₁-C₅alkyl)₂aminopropyloxy group, a (C₁-C₅alkyl)₂aminobutyloxy group, or a (C₁-C₅alkyl)₂aminopentyloxy group. The C₁-C₅alkyl substituents at the amino group may be connected so as to form a ring. For example, the (C₁-C₅alkyl)₂aminoethyloxy group may be a N,N-dimethylaminoethyloxy group or pyrrolidinethyloxy group. In another example, the (C₁-C₅alkyl)₂aminopropyloxy group may be a N,N-dimethylaminopropyloxy group or pyrrolidinpropyloxy group. In another embodiment, R⁴ may be a heterocyclyl(C₂-C₅)alkyloxy group or a heteroaryl(C₂-C₅)alkyloxy group, wherein “heterocyclyl” is a substituted or unsubstituted nitrogen-containing non-aromatic mono-heterocycle and wherein “heteroaryl” is a substituted or substituted nitrogen-containing aromatic mono-heterocycle. Both heterocyclyl and heteroaryl groups may be connected to the “(C₂-C₅)alkyloxy” through a nitrogen atom. Examples of substituted or unsubstituted “heterocyclyl” and “heteroaryl” groups are known to one of ordinary skill in the art and some of them are recited in the disclosure above. An example of the “heterocyclyl” group may be a substituted or unsubstituted piperazine group. An example of the “heteroaryl” group may be a substituted or unsubstituted pyrazole group.

In another embodiment of Formula (I), R⁴ may be a (C₀-C₅alkyl)₂amino(C₂-C₅)alkyl group. The description of the (C₀-C₅alkyl)₂amino(C₂-C₅)alkyl group is the same as the (C₀-C₅alkyl)₂amino(C₂-C₅)alkyloxy group above, so it will not be repeated here. In another embodiment, R⁴ may be a heterocyclyl(C₂-C₅)alkyl group or a heteroaryl(C₂-C₅)alkyl group.

The description of the heterocyclyl(C₂-C₅)alkyl group or a heteroaryl(C₂-C₅)alkyl group is the same as the heterocyclyl(C₂-C₅)alkyloxy group or a heteroaryl(C₂-C₅)alkyloxy group above, so it will not be repeated here.

In another embodiment, disclosed is a method for treating or preventing a disease that involves TDP-43, the method including administering to a subject an effective amount of at least one compound having formula (II):

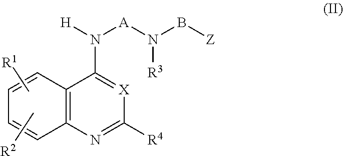

an enantiomer, a diastereomer, a hydrate, a solvate, a pharmaceutically acceptable salt, an isotopic analog, a prodrug or a complex thereof,

wherein, in formula (II):

X is N or CH;

R¹ and R² are the same or different and are each independently H, halogen, C₁-C₄ alkyl, C₁-C₄ alkoxy, CF₃, or CF₃O, wherein C₁-C₄ alkyl and C₁-C₄ alkoxy are optionally with at least one fluorine;

R³ is H, C₁-C₇ alkyl, C₄-C₇ carbocyclic, aryl, heteroaryl, heterocyclic, heterocyclic alkyl, wherein R³ is optionally taken together with either A or B to form a ring consisting of 4-7 ring members;

R⁴=phenyl or phenyl substituted with C₁-C₄ alkyl, C₁-C₄ alkoxy, CF₃, CF₃O, halogen, amino, or sulfonamide;

A=(CH₂)ₙ, wherein n is 2-4, wherein A and R³ are optionally taken together to form a ring consisting of 4-7 ring members;

B=(CH₂)ₙ, wherein n is 2-6, wherein B and R³ are optionally taken together to form a ring consisting of 4-7 members, and wherein B is optionally substituted with one or more substituents selected from C₁-C₄ alkyl, hydroxy, C₁-C₄ alkoxy, CF₃, and CF₃O; and

Z is OH, C₁-C₄ alkoxy, OCF₃, or N with one or more of H, C₁-C₄ alkyl, heteroaryl, substituted heteroaryl, C₁-C₄ sulfonamido, substituted amido.

In another embodiment, disclosed is a method for treating or preventing a disease that involves TDP-43, the method including administering to a subject an effective amount of at least one compound having formulae (IIIa) or (IIIb):

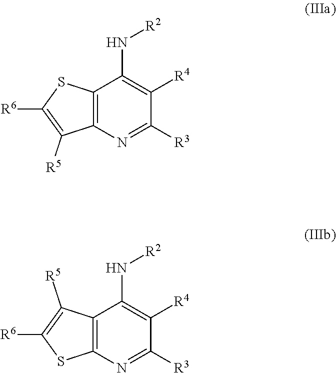

an enantiomer, a diastereomer, a hydrate, a solvate, a pharmaceutically acceptable salt, an isotopic analog, a prodrug or a complex thereof,

wherein, in formulae (IIIa) and (IIIb):

R² is a substituted or unsubstituted C₁-C₂₀ linear, branched, or cyclic organic group including at least one nitrogen; and

R³, R⁴, R⁵, and R⁶ are each independently hydrogen, halogen, a hydroxyl group, a cyano group, a nitro group, a substituted or unsubstituted C₀-C₁₀ amino group, a substituted or unsubstituted C₁-C₁₀ alkyl group, a substituted or unsubstituted C₂-C₁₀ alkenyl group, a substituted or unsubstituted C₂-C₁₀ alkynyl group, and a substituted or unsubstituted C₁-C₁₀ alkoxy group, a substituted or unsubstituted C₃-C₁₀ cycloalkyl group, a substituted or unsubstituted C₂-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₂₀ aryl group, a substituted or unsubstituted C₆-C₂₀ aryloxy group, a substituted or unsubstituted C₆-C₂₀ arylthio group, a substituted or unsubstituted C₂-C₂₀ heteroaryl group, a substituted or unsubstituted C₂-C₂₀ heteroaryloxy group, or a substituted or unsubstituted C₂-C₂₀ heteroarylthio group.

In another embodiment, disclosed is a method for treating or preventing a disease that involves TDP-43, the method including administering to a subject an effective amount of at least one compound having formula (IV):

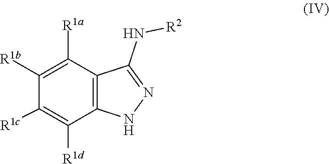

an enantiomer, a diastereomer, a hydrate, a solvate, a pharmaceutically acceptable salt, an isotopic analog, a prodrug or a complex thereof,

wherein, in formula (IV):

R² is a substituted or unsubstituted C₁-C₂₀ linear, branched, or cyclic organic group including at least one nitrogen; and

R^{1a}, R^{1l}, R^{1c}, and R^{1d} are each independently hydrogen, halogen, a hydroxyl group, a cyano group, a nitro group, a substituted or unsubstituted C₀-C₁₀ amino group, a substituted or unsubstituted C₁-C₁₀ alkyl group, a substituted or unsubstituted C₂-C₁₀ alkenyl group, a substituted or unsubstituted C₂-C₁₀ alkynyl group, and a substituted or unsubstituted C₁-C₁₀ alkoxy group, a substituted or unsubstituted C₃-C₁₀ cycloalkyl group, a substituted or unsubstituted C₂-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₂₀ aryl group, a substituted or unsubstituted C₆-C₂₀ aryloxy group, a substituted or unsubstituted C₆-C₂₀ arylthio group, a substituted or unsubstituted C₂-C₂₀ heteroaryl group, a substituted or unsubstituted C₂-C₂₀ heteroaryloxy group, or a substituted or unsubstituted C₂-C₂₀ heteroarylthio group.

In another embodiment, disclosed is a method for treating or preventing a disease that involves TDP-43, the method including administering to a subject an effective amount of at least one compound having formula (V):

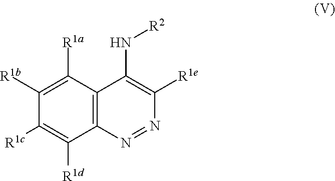

an enantiomer, a diastereomer, a hydrate, a solvate, a pharmaceutically acceptable salt, an isotopic analog, a prodrug or a complex thereof,

wherein, in formula (IV):

R² is a substituted or unsubstituted C₁-C₂₀ linear, branched, or cyclic organic group including at least one nitrogen; and

R^{1a}, R^{1l}, R^{1c}, R^{1d}, and R^{1c} are each independently hydrogen, halogen, a hydroxyl group, a cyano group, a nitro group, a substituted or unsubstituted C₀-C₁₀ amino group, a substituted or unsubstituted C₁-C₁₀ alkyl group, a substituted or unsubstituted C₂-C₁₀ alkenyl group, a substituted or unsubstituted C₂-C₁₀ alkynyl group, and a substituted or unsubstituted C₁-C₁₀ alkoxy group, a substituted or unsubstituted C₃-C₁₀ cycloalkyl group, a substituted or unsubstituted C₂-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₂₀ aryl group, a substituted or unsubstituted C₆-C₂₀ aryloxy group, a substituted or unsubstituted C₆-C₂₀ arylthio group, a substituted or unsubstituted C₂-C₂₀ heteroaryl group, a substituted or unsubstituted C₂-C₂₀ heteroaryloxy group, or a substituted or unsubstituted C₂-C₂₀ heteroarylthio group.

Embodiments of the present invention further relate to compositions including an effective amount of one or more compounds according to the present invention and an excipient.

Embodiments of the present invention also relates to a method for treating or preventing diseases that involve TDP-43, including, for example, ALS, FTLD, CTE, hippocampal sclerosis of aging (CARTS), Alzheimer's disease, or an Alzheimer's disease related disorder, said method including administering to a subject an effective amount of a compound or composition according to the present invention.

Embodiments of the present invention yet further relates to a method for treating or preventing diseases that involve TDP-43, including, for example, ALS, FTLD, CTE, hippocampal sclerosis of aging (CARTS), Alzheimer's disease, or an Alzheimer's disease related disorder, wherein said method includes administering to a subject a composition including an effective amount of one or more compounds according to the present invention and an excipient.

Embodiments of the present invention yet further relates to a method for treating or preventing diseases that involve TDP-43, including, for example, ALS, FTLD, CTE, hippocampal sclerosis of aging (CARTS), Alzheimer's disease, or an Alzheimer's disease related disorder, wherein said method includes administering to a subject a composition including an effective amount of one or more compounds according to the present invention and an excipient, and on or more compounds selected from the group consisting of riluzole, troriluzole (trigriluzole), and edavarone.

Embodiments of the present invention also relates to a method for treating or preventing disease or conditions associated with TDP-43 proteinopathies, and diseases that involve excess amounts of TDP-43 in the cytosol. Said methods include administering to a subject an effective amount of a compound or composition according to the present invention.

Embodiments of the present invention yet further relates to a method for treating or preventing disease or conditions associated with TDP-43 proteinopathies, and diseases that involve excess amounts of TDP-43 in the cytosol, wherein said method includes administering to a subject a composition including an effective amount of one or more compounds according to the present invention and an excipient.

Embodiments of the present invention yet further relates to a method for treating or preventing disease or conditions associated with TDP-43 proteinopathies, and diseases that involve excess amounts of TDP-43 in the cytosol, wherein said method includes administering to a subject a composition including an effective amount of one or more compounds according to the present invention and an excipient, and one or more compounds selected from the group consisting of riluzole, troriluzole (trigriluzole), and edavarone.

Embodiments of the present invention also relates to a method for treating or preventing disease or conditions associated with TDP-43. Said methods include administering to a subject an effective amount of a compound or composition according to the present invention.

Embodiments of the present invention yet further relates to a method for treating or preventing disease or conditions associated with TDP-43, wherein said method includes administering to a subject a composition including an effective amount of one or more compounds according to the present invention and an excipient.

Embodiments of the present invention yet further relates to a method for treating or preventing disease or conditions associated with TDP-43, wherein said method includes administering to a subject a composition including an effective amount of one or more compounds according to the present invention and an excipient, and on or more compounds selected from the group consisting of riluzole, troriluzole, (trigriluzole), and edavarone.

Embodiments of the present invention yet further relates to the method of use of the TDP-43 binders of the present invention as positron emission tomography (PET) imaging agents, wherein said method includes administering to a subject an effective amount of an isotopically labeled compound or composition according to the present invention.

Embodiments of the present invention yet further relates to the method of use of the TDP-43 binders of the present invention as single-photon emission computed tomography (SPECT) imaging agents, wherein said method includes administering to a subject an effective amount of an isotopically labeled compound or composition according to the present invention.

Embodiments of the present invention further relates to a process for preparing the TDP-43 binders of the present invention.

These and other objects, features, and advantages will become apparent to those of ordinary skill in the art from a reading of the following detailed description and the appended claims. All percentages, ratios and proportions herein are by weight, unless otherwise specified. All temperatures are in degrees Celsius (° C.) unless otherwise specified. All documents cited are in relevant part, incorporated herein by reference; the citation of any document is not to be construed as an admission that it is prior art with respect to the present invention.

The TDP-43 binders of the present invention are capable of treating and preventing diseases associated with TDP-43, for example ALS, FTLD, CTE, hippocampal sclerosis of aging (CARTS), Alzheimer's disease, and Alzheimer's disease related disorders. In addition, the TDP-43 binders of the present invention are also useful as positron emission tomography (PET) imaging agents, useful for the diagnosis of diseases and conditions associated with TDP-43. Further, the TDP-43 binders of the present invention are also useful as single-photon emission computed tomography (SPECT) imaging agents, useful for the diagnosis of diseases and conditions associated with TDP-43.

Throughout the description, where compositions are described as having, including, or including specific components, or where processes are described as having, including, or including specific process steps, it is contemplated that compositions of the present teachings also consist essentially of, or consist of, the recited components, and that the processes of the present teachings also consist essentially of, or consist of, the recited processing steps.

In the application, where an element or component is said to be included in and/or selected from a list of recited elements or components, it should be understood that the element or component can be any one of the recited elements or components and can be selected from a group consisting of two or more of the recited elements or components.

The use of the singular herein includes the plural (and vice versa) unless specifically stated otherwise. In addition, where the use of the term “about” is before a quantitative value, the present teachings also include the specific quantitative value itself, unless specifically stated otherwise.

It should be understood that the order of steps or order for performing certain actions is immaterial so long as the present teachings remain operable. Moreover, two or more steps or actions can be conducted simultaneously.

As used herein, the term “halogen” shall mean chlorine, bromine, fluorine and iodine.

As used herein, unless otherwise noted, “alkyl” and/or “aliphatic” whether used alone or as part of a substituent group refers to straight and branched carbon chains having 1 to 20 carbon atoms or any number within this range, for example 1 to 6 carbon atoms or 1 to 4 carbon atoms.

Designated numbers of carbon atoms (e.g., C₁.₆) shall refer independently to the number of carbon atoms in an alkyl moiety or to the alkyl portion of a larger alkyl-containing substituent. Non-limiting examples of alkyl groups include methyl, ethyl, n-propyl, iso-propyl, n-butyl, sec-butyl, iso-butyl, tert-butyl, and the like. Alkyl groups can be optionally substituted. Non-limiting examples of substituted alkyl groups include hydroxymethyl, chloromethyl, trifluoromethyl, aminomethyl, 1-chloroethyl, 2-hydroxyethyl, 1,2-difluoroethyl, 3-carboxypropyl, and the like. In substituent groups with multiple alkyl groups such as (C₁₋₆alkyl)₂amino, the alkyl groups may be the same or different.

As used herein, unless otherwise noted, “heteroalkyl” whether used alone or as part of a substituent group refers to an “alkyl” group as defined above in which at least one carbon atom is replaced with a heteroatom selected from the group consisting of nitrogen (N), oxygen (O), sulfur (S), and phosphorus (P). Non-limiting examples of alkyl groups include methoxy, ethoxy, methoxyethyl, methoxyethoxy, dimethylaminoethyl, dimethylaminopropyl, diethylaminoethyl, diethylaminopropyl, isopropylaminopropyl, azetidinopropyl, pyrrolidinopropyl, piperidinopropyl, pyrrolidinopropoxy, piperidinopropoxy, and the like.

As used herein, the terms “alkenyl” and “alkynyl” groups, whether used alone or as part of a substituent group, refer to straight and branched carbon chains having 2 or more carbon atoms, preferably 2 to 20, wherein an alkenyl chain has at least one double bond in the chain and an alkynyl chain has at least one triple bond in the chain. Alkenyl and alkynyl groups can be optionally substituted. Non-limiting examples of alkenyl groups include ethenyl, 3-propenyl, 1-propenyl (also 2-methylethenyl), isopropenyl (also 2-methylethen-2-yl), buten-4-yl, and the like.

Non-limiting examples of substituted alkenyl groups include 2-chloroethenyl (also 2-chlorovinyl), 4-hydroxybuten-1-yl, 7-hydroxy-7-methyloct-4-en-2-yl, 7-hydroxy-7-methyloct-3,5-dien-2-yl, and the like. Non-limiting examples of alkynyl groups include ethynyl, prop-2-ynyl (also propargyl), propyn-1-yl, and 2-methyl-hex-4-yn-1-yl. Non-limiting examples of substituted alkynyl groups include, 5-hydroxy-5-methylhex-3-ynyl, 6-hydroxy-6-methylhept-3-yn-2-yl, 5-hydroxy-5-ethylhept-3-ynyl, and the like.

As used herein, “cycloalkyl,” whether used alone or as part of another group, refers to a non-aromatic carbon-containing ring including cyclized alkyl, alkenyl, and alkynyl groups, e.g., having from 3 to 14 ring carbon atoms, preferably from 3 to 7 or 3 to 6 ring carbon atoms, or even 3 to 4 ring carbon atoms, and optionally containing one or more (e.g., 1, 2, or 3) double or triple bond. Cycloalkyl groups can be monocyclic (e.g., cyclohexyl) or polycyclic (e.g., containing fused, bridged, and/or spiro ring systems), wherein the carbon atoms are located inside or outside of the ring system. Any suitable ring position of the cycloalkyl group can be covalently linked to the defined chemical structure. Cycloalkyl rings can be optionally substituted. Non-limiting examples of cycloalkyl groups include: cyclopropyl, 2-methyl-cyclopropyl, cyclopropenyl, cyclobutyl, 2,3-dihydroxycyclobutyl, cyclobutenyl, cyclopentyl, cyclopentenyl, cyclopentadienyl, cyclohexyl, cyclohexenyl, cycloheptyl, cyclooctanyl, decalinyl, 2,5-dimethylcyclopentyl, 3,5-dichlorocyclohexyl, 4-hydroxycyclohexyl, 3,3,5-trimethylcyclohex-1-yl, octahydropentalenyl, octahydro-1H-indenyl, 3a,4,5,6,7,7a-hexahydro-3H-inden-4-yl, decahydroazulenyl; bicyclo[6.2.0]decanyl, decahydronaphthalenyl, and dodecahydro-1H-fluorenyl. The term “cycloalkyl” also includes carbocyclic rings which are bicyclic hydrocarbon rings, non-limiting examples of which include, bicyclo-[2.1.1]hexanyl, bicyclo[2.2.1]heptanyl, bicyclo[3.1.1]heptanyl, 1,3-dimethyl[2.2.1]heptan-2-yl, bicyclo[2.2.2]octanyl, and bicyclo[3.3.3]undecanyl.

“Haloalkyl” is intended to include both branched and straight-chain saturated aliphatic hydrocarbon groups having the specified number of carbon atoms, substituted with 1 or more halogen. Haloalkyl groups include perhaloalkyl groups, wherein all hydrogens of an alkyl group have been replaced with halogens (e.g., —CF₃, —CF₂CF₃). Haloalkyl groups can optionally be substituted with one or more substituents in addition to halogen. Examples of haloalkyl groups include, but are not limited to, fluoromethyl, dichloroethyl, trifluoromethyl, trichloromethyl, pentafluoroethyl, and pentachloroethyl groups.

The term “alkoxy” refers to the group —O-alkyl, wherein the alkyl group is as defined above. Alkoxy groups optionally may be substituted. The term C₃-C₆ cyclic alkoxy refers to a ring containing 3 to 6 carbon atoms and at least one oxygen atom (e.g., tetrahydrofuran, tetrahydro-2H-pyran). C₃-C₆ cyclic alkoxy groups optionally may be substituted.

The term “aryl,” wherein used alone or as part of another group, is defined herein as an unsaturated, aromatic monocyclic ring of 6 carbon members or to an unsaturated, aromatic polycyclic ring of from 10 to 14 carbon members. Aryl rings can be, for example, phenyl or naphthyl ring each optionally substituted with one or more moieties capable of replacing one or more hydrogen atoms. Non-limiting examples of aryl groups include: phenyl, naphthylen-1-yl, naphthylen-2-yl, 4-fluorophenyl, 2-hydroxyphenyl, 3-methylphenyl, 2-amino-4-fluorophenyl, 2-(N,N-diethylamino)phenyl, 2-cyanophenyl, 2,6-di-tert-butylphenyl, 3-methoxyphenyl, 8-hydroxynaphthylen-2-yl 4,5-dimethoxynaphthylen-1-yl, and 6-cyano-naphthylen-1-yl. Aryl groups also include, for example, phenyl or naphthyl rings fused with one or more saturated or partially saturated carbon rings (e.g., bicyclo[4.2.0]octa-1,3,5-trienyl, indanyl), which can be substituted at one or more carbon atoms of the aromatic and/or saturated or partially saturated rings.

The term “arylalkyl” or “aralkyl” refers to the group -alkyl-aryl, where the alkyl and aryl groups are as defined herein. Aralkyl groups of the present invention are optionally substituted.

Examples of arylalkyl groups include, for example, benzyl, 1-phenylethyl, 2-phenylethyl, 3-phenylpropyl, 2-phenylpropyl, fluorenylmethyl and the like.

The terms “heterocyclic” and/or “heterocycle” and/or “heterocyclyl,” whether used alone or as part of another group, are defined herein as one or more ring having from 3 to 20 atoms wherein at least one atom in at least one ring is a heteroatom selected from nitrogen (N), oxygen (O), or sulfur (S), and wherein further the ring that includes the heteroatom is non-aromatic. In heterocycle groups that include 2 or more fused rings, the non-heteroatom bearing ring may be aryl (e.g., indolinyl, tetrahydroquinolinyl, chromanyl). Exemplary heterocycle groups have from 3 to 14 ring atoms of which from 1 to 5 are heteroatoms independently selected from nitrogen (N), oxygen (O), or sulfur (S). One or more N or S atoms in a heterocycle group can be oxidized.

Heterocycle groups can be optionally substituted.

Non-limiting examples of heterocyclic units having a single ring include: diazirinyl, aziridinyl, urazolyl, azetidinyl, pyrazolidinyl, imidazolidinyl, oxazolidinyl, isoxazolinyl, isoxazolyl, thiazolidinyl, isothiazolyl, isothiazolinyl oxathiazolidinonyl, oxazolidinonyl, hydantoinyl, tetrahydrofuranyl, pyrrolidinyl, morpholinyl, piperazinyl, piperidinyl, dihydropyranyl, tetrahydropyranyl, piperidin-2-onyl (valerolactam), 2,3,4,5-tetrahydro-1H-azepinyl, 2,3-dihydro-1H-indole, and 1,2,3,4-tetrahydro-quinoline. Non-limiting examples of heterocyclic units having 2 or more rings include: hexahydro-1H-pyrrolizinyl, 3a,4,5,6,7,7a-hexahydro-1H-benzo[d]imidazolyl, 3a,4,5,6,7,7a-hexahydro-1H-indolyl, 1,2,3,4-tetrahydroquinolinyl, chromanyl, isochromanyl, indolinyl, isoindolinyl, and decahydro-1H-cycloocta[b]pyrrolyl.

The term “heteroaryl,” whether used alone or as part of another group, is defined herein as one or more rings having from 5 to 20 atoms wherein at least one atom in at least one ring is a heteroatom chosen from nitrogen (N), oxygen (O), or sulfur (S), and wherein further at least one of the rings that includes a heteroatom is aromatic. In heteroaryl groups that include 2 or more fused rings, the non-heteroatom bearing ring may be a carbocycle (e.g., 6,7-Dihydro-5H-cyclopentapyrimidine) or aryl (e.g., benzofuranyl, benzothiophenyl, indolyl). Exemplary heteroaryl groups have from 5 to 14 ring atoms and contain from 1 to 5 ring heteroatoms independently selected from nitrogen (N), oxygen (O), or sulfur (S). One or more N or S atoms in a heteroaryl group can be oxidized. Heteroaryl groups can be substituted. Non-limiting examples of heteroaryl rings containing a single ring include: 1,2,3,4-tetrazolyl, [1,2,3]triazolyl, [1,2,4]triazolyl, triazinyl, thiazolyl, 1H-imidazolyl, oxazolyl, furanyl, thiopheneyl, pyrimidinyl, 2-phenylpyrimidinyl, pyridinyl, 3-methylpyridinyl, and 4-dimethylaminopyridinyl. Non-limiting examples of heteroaryl rings containing 2 or more fused rings include: benzofuranyl, benzothiophenyl, benzoxazolyl, benzthiazolyl, benztriazolyl, cinnolinyl, naphthyridinyl, phenanthridinyl, 7H-purinyl, 9H-purinyl, 6-amino-9H-purinyl, 5H-pyrrolo[3,2-d]pyrimidinyl, 7H-pyrrolo[2,3-d]pyrimidinyl, pyrido[2,3-d]pyrimidinyl, 2-phenylbenzo[d]thiazolyl, 1H-indolyl, 4,5,6,7-tetrahydro-1-H-indolyl, quinoxalinyl, 5-methylquinoxalinyl, quinazolinyl, quinolinyl, 8-hydroxy-quinolinyl, and isoquinolinyl.

One non-limiting example of a heteroaryl group as described above is C₁-C₅ heteroaryl, which has 1 to 5 carbon ring atoms and at least one additional ring atom that is a heteroatom (preferably 1 to 4 additional ring atoms that are heteroatoms) independently selected from nitrogen (N), oxygen (O), or sulfur (S). Examples of C₁-C₅ heteroaryl include, but are not limited to, triazinyl, thiazol-2-yl, thiazol-4-yl, imidazol-1-yl, 1H-imidazol-2-yl, 1H-imidazol-4-yl, isoxazolin-5-yl, furan-2-yl, furan-3-yl, thiophen-2-yl, thiophen-4-yl, pyrimidin-2-yl, pyrimidin-4-yl, pyrimidin-5-yl, pyridin-2-yl, pyridin-3-yl, and pyridin-4-yl.

Unless otherwise noted, when two substituents are taken together to form a ring having a specified number of ring atoms (e.g., R² and R³ taken together with the nitrogen (N) to which they are attached to form a ring having from 3 to 7 ring members), the ring can have carbon atoms and optionally one or more (e.g., 1 to 3) additional heteroatoms independently selected from nitrogen (N), oxygen (O), or sulfur (S). The ring can be saturated or partially saturated and can be optionally substituted.

For the purposed of the present invention fused ring units, as well as spirocyclic rings, bicyclic rings and the like, which include a single heteroatom will be considered to belong to the cyclic family corresponding to the heteroatom containing ring. For example, 1,2,3,4-tetrahydroisoquinoline having the formula:

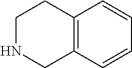

is, for the purposes of the present invention, considered a heterocyclic unit. For example 2,4,5,6-tetrahydropyrrolo[3,4-c]pyrazole having the formula:

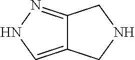

is, for the purposes of the present invention, considered a heterocyclic unit.

Whenever a term or either of their prefix roots appear in a name of a substituent the name is to be interpreted as including those limitations provided herein. For example, whenever the term “alkyl” or “aryl” or either of their prefix roots appear in a name of a substituent (e.g., arylalkyl, alkylamino) the name is to be interpreted as including those limitations given above for “alkyl” and “aryl.”

The term “substituted” is used throughout the specification. The term “substituted” is defined herein as a moiety, whether acyclic or cyclic, which has one or more hydrogen atoms replaced by a substituent or several (e.g., 1 to 10) substituents as defined herein below. The substituents are capable of replacing one or two hydrogen atoms of a single moiety at a time. In addition, these substituents can replace two hydrogen atoms on two adjacent carbons to form said substituent, new moiety or unit. For example, a substituted unit that requires a single hydrogen atom replacement includes halogen, hydroxyl, and the like. A two hydrogen atom replacement includes carbonyl, oximino, and the like. A two hydrogen atom replacement from adjacent carbon atoms includes epoxy, and the like. The term “substituted” is used throughout the present specification to indicate that a moiety can have one or more of the hydrogen atoms replaced by a substituent. When a moiety is described as “substituted” any number of the hydrogen atoms may be replaced. For example, difluoromethyl is a substituted C₁ alkyl; trifluoromethyl is a substituted C₁ alkyl; 4-hydroxyphenyl is a substituted aromatic ring; (N,N-dimethyl-5-amino)octanyl is a substituted C₈ alkyl; 3-guanidinopropyl is a substituted C₃ alkyl; and 2-carboxypyridinyl is a substituted heteroaryl.

The variable groups defined herein, e.g., alkyl, alkenyl, alkynyl, cycloalkyl, alkoxy, aryloxy, aryl, heterocycle and heteroaryl groups defined herein, whether used alone or as part of another group, can be optionally substituted. Optionally substituted groups will be so indicated.

The following are non-limiting examples of substituents which can substitute for hydrogen atoms on a moiety: halogen (chlorine (C₁), bromine (Br), fluorine (F) and iodine(I)), —CN, —NO₂, oxo (═O), —OR²³, —SR²³, —N(R²³)₂, —NR²³C(O)R²³, —SO₂R²³, —SO₂OR²³, —SO₂N(R²³)₂, —C(O)R²³, —C(O)OR²³, —C(O)N(R²³)₂, C₁₋₆ alkyl, C₁₋₆ haloalkyl, C₁₋₆ alkoxy, C₂₋ₛ alkenyl, C₂₋₈ alkynyl, C₃₋₁₄ cycloalkyl, aryl, heterocycle, or heteroaryl, wherein each of the alkyl, haloalkyl, alkenyl, alkynyl, alkoxy, cycloalkyl, aryl, heterocycle, and heteroaryl groups is optionally substituted with 1-10 (e.g., 1-6 or 1-4) groups selected independently from halogen, —CN, —NO₂, oxo, and R²³; wherein R²³, at each occurrence, independently is hydrogen, —OR²⁴, —SR²⁴, —C(O)R²⁴, —C(O)OR²⁴, —C(O)N(R²⁴)₂, —SO₂R²⁴, —S(O)₂OR²⁴, —N(R²⁴)₂, —NR²⁴C(O)R²⁴, C₁₋₆ alkyl, C₁₋₆ haloalkyl, C₂₋₈ alkenyl, C₂₋₈ alkynyl, cycloalkyl (e.g., C₃₋₆ cycloalkyl), aryl, heterocycle, or heteroaryl, or two R²³ units taken together with the atom(s) to which they are bound form an optionally substituted carbocycle or heterocycle wherein said carbocycle or heterocycle has 3 to 7 ring atoms; wherein R²⁴, at each occurrence, independently is hydrogen, C₁₋₆ alkyl, C₁₋₆ haloalkyl, C₂₋₈ alkenyl, C₂₋₈ alkynyl, cycloalkyl (e.g., C₃₋₆ cycloalkyl), aryl, heterocycle, or heteroaryl, or two R²⁴ units taken together with the atom(s) to which they are bound form an optionally substituted carbocycle or heterocycle wherein said carbocycle or heterocycle preferably has 3 to 7 ring atoms.

In some embodiments, the substituents are selected from
i) —OR²⁵; for example, —OH, —OCH₃, —OCH₂CH₃, —OCH₂CH₂CH₃;
ii) —C(O)R²⁵; for example, —COCH₃, —COCH₂CH₃, —COCH₂CH₂CH₃;
iii) —C(O)OR²⁵; for example, —CO₂CH₃, —CO₂CH₂CH₃, —CO₂CH₂CH₂CH₃;
iv) —C(O)N(R²⁵)₂; for example, —CONH₂, —CONHCH₃, —CON(CH₃)₂;
v) —N(R²⁵)₂; for example, —NH₂, —NHCH₃, —N(CH₃)₂, —NH(CH₂CH₃);
vi) halogen: —F, —Cl, —Br, and —I;
vii) —CHₑX_{g}; wherein X is halogen, e and g are each from 0 to 2, provided that e+g=3; for example, —CH₂F, —CHF₂, —CF₃, —CCl₃, or —CBr₃;
viii) —SO₂R²⁵; for example, —SO₂H; —SO₂CH₃; —SO₂C₆H₅;
ix) C₁-C₆ linear, branched, or cyclic alkyl;
x) cyano
xi) nitro;
xii) N(R²⁵)C(O)R²⁵;
xiii) oxo (═O);
xiv) heterocycle; and
xv) heteroaryl,

wherein each R²⁵ is independently hydrogen, optionally substituted C₁-C₆ linear or branched alkyl (e.g., optionally substituted C₁-C₄ linear or branched alkyl), or optionally substituted C₃-C₆ cycloalkyl (e.g., optionally substituted C₃-C₄ cycloalkyl); or two R²⁵ units can be taken together to form a ring including 3-7 ring atoms. In certain aspects, each R²⁵ is independently hydrogen, C₁-C₆ linear or branched alkyl optionally substituted with halogen or C₃-C₆ cycloalkyl or C₃-C₆ cycloalkyl.

At various places in the present specification, substituents of compounds are disclosed in groups or in ranges. It is specifically intended that the description include each and every individual subcombination of the members of such groups and ranges. For example, the term “C₁₋₆alkyl” is specifically intended to individually disclose C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆, alkyl.

For the purposes of the present invention the terms “compound,” “analog,” and “composition of matter” stand equally well for the TDP-43 binders described herein, including all enantiomeric forms, diastereomeric forms, salts, and the like, and the terms “compound,” “analog,” and “composition of matter” are used interchangeably throughout the present specification.

Compounds described herein can contain an asymmetric atom (also referred as a chiral center), and some of the compounds can contain one or more asymmetric atoms or centers, which can thus give rise to optical isomers (enantiomers) and diastereomers. The present teachings and compounds disclosed herein include such enantiomers and diastereomers, as well as the racemic and resolved, enantiomerically pure R and S stereoisomers, as well as other mixtures of the R and S stereoisomers and pharmaceutically acceptable salts thereof. Optical isomers can be obtained in pure form by standard procedures known to those skilled in the art, which include, but are not limited to, diastereomeric salt formation, kinetic resolution, and asymmetric synthesis. The present teachings also encompass cis and trans isomers of compounds containing alkenyl moieties (e.g., alkenes and imines). It is also understood that the present teachings encompass all possible regioisomers, and mixtures thereof, which can be obtained in pure form by standard separation procedures known to those skilled in the art, and include, but are not limited to, column chromatography, thin-layer chromatography, and high-performance liquid chromatography.

Pharmaceutically acceptable salts of compounds of the present teachings, which can have an acidic moiety, can be formed using organic and inorganic bases. Both mono and polyanionic salts are contemplated, depending on the number of acidic hydrogens available for deprotonation. Suitable salts formed with bases include metal salts, such as alkali metal or alkaline earth metal salts, for example sodium, potassium, or magnesium salts; ammonia salts and organic amine salts, such as those formed with morpholine, thiomorpholine, piperidine, pyrrolidine, a mono-, di- or tri-lower alkylamine (e.g., ethyl-tert-butyl-, diethyl-, diisopropyl-, triethyl-, tributyl- or dimethylpropylamine), or a mono-, di-, or trihydroxy lower alkylamine (e.g., mono-, di- or triethanolamine). Specific non-limiting examples of inorganic bases include NaHCO₃, Na₂CO₃, KHCO₃, K₂CO₃, Cs₂CO₃, LiGH, NaOH, KOH, NaH₂PO₄, Na₂HPO₄, and Na₃PO₄. Internal salts also can be formed. Similarly, when a compound disclosed herein contains a basic moiety, salts can be formed using organic and inorganic acids. For example, salts can be formed from the following acids: acetic, propionic, lactic, benzenesulfonic, benzoic, camphorsulfonic, citric, tartaric, succinic, dichloroacetic, ethenesulfonic, formic, fumaric, gluconic, glutamic, hippuric, hydrobromic, hydrochloric, isethionic, lactic, maleic, malic, malonic, mandelic, methanesulfonic, mucic, napthalenesulfonic, nitric, oxalic, pamoic, pantothenic, phosphoric, phthalic, propionic, succinic, sulfuric, tartaric, toluenesulfonic, and camphorsulfonic as well as other known pharmaceutically acceptable acids.

When any variable occurs more than one time in any constituent or in any formula, its definition in each occurrence is independent of its definition at every other occurrence (e.g., in N(R²⁴)₂, each R²⁴ may be the same or different than the other). Combinations of substituents and/or variables are permissible only if such combinations result in stable compounds

The terms “treat” and “treating” and “treatment” as used herein, refer to partially or completely alleviating, inhibiting, ameliorating and/or relieving a condition from which a patient is suspected to suffer.

As used herein, “therapeutically effective” and “effective dose” refer to a substance or an amount that elicits a desirable biological activity or effect.

Except when noted, the terms “subject” or “patient” are used interchangeably and refer to mammals such as human patients and non-human primates, as well as experimental animals such as rabbits, rats, and mice, and other animals. Accordingly, the term “subject” or “patient” as used herein means any mammalian patient or subject to which the compounds of the invention can be administered. In an exemplary embodiment of the present invention, to identify subject patients for treatment according to the methods of the invention, accepted screening methods are employed to determine risk factors associated with a targeted or suspected disease or condition or to determine the status of an existing disease or condition in a subject. These screening methods include, for example, conventional work-ups to determine risk factors that may be associated with the targeted or suspected disease or condition. These and other routine methods allow the clinician to select patients in need of therapy using the methods and compounds of the present invention.

The TDP-43 binders of the present invention are compounds, which include all enantiomeric and diastereomeric forms and pharmaceutically accepted salts thereof. The TDP-43 binders of the present invention may include a compound having the formula (I):

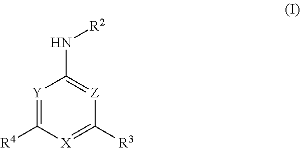

including an enantiomer, a diastereomer, a hydrate, a solvate, a pharmaceutically acceptable salt, an isotopic analog, a prodrug, and a complex thereof.

In formula (I), R² is a C₁-C₂₀ organic group including at least one nitrogen atom. The C₁-C₂₀ organic group may be linear, branched, or cyclic, and may be either substituted or unsubstituted. The C₁-C₂₀ organic group may be a substituted or unsubstituted C₁-C₂₀ hydrocarbon group, in which at least one carbon atom is replaced by nitrogen. The C₁-C₂₀ organic group including at least one nitrogen atom may include at least one C₀-C₁₀ amino group, which may be primary, secondary, or tertiary, and which may be monocyclic, bicyclic, or tricyclic. The C₁-C₂₀ organic group may include a C₄-C₈ monocyclic amino group, a C₄-C₁₀ bicyclic amino group, or a C₆-C₁₂ triyclic amino group.

In an embodiment, at least one of the substituents of the C₁-C₂₀ heteroalkyl group may be selected from: —F, —Cl, —Br, —I, a hydroxyl group, a cyano group, a nitro group, a C₀-C₁₀ amino group, a C₁-C₁₀ alkyl group, a C₂-C₁₀ alkenyl group, a C₂-C₁₀ alkynyl group, and a C₁-C₁₀ alkoxy group; a C₁-C₁₀ alkyl group, a C₂-C₁₀ alkenyl group, a C₂-C₁₀ alkynyl group, and a C₁-C₁₀ alkoxy group, each substituted with at least one selected from —F, —Cl, —Br, —I, a hydroxyl group, a cyano group, a nitro group, a C₀-C₁₀ amino group, a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₂₀ aryl group, a C₆-C₂₀ aryloxy group, a C₆-C₂₀ arylthio group, a C₂-C₂₀ heteroaryl group, a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₂₀ aryl group, a C₆-C₂₀ aryloxy group, a C₆-C₂₀ arylthio group, a C₂-C₂₀ heteroaryl group, and a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₂₀ aryl group, a C₆-C₂₀ aryloxy group, a C₆-C₂₀ arylthio group, a C₂-C₂₀ heteroaryl group, each substituted with at least one selected from —F, —Cl, —Br, —I, a hydroxyl group, a cyano group, a nitro group, a C₀-C₁₀ amino group, a C₁-C₁₀ alkyl group, a C₂-C₁₀ alkenyl group, a C₂-C₁₀ alkynyl group, a C₁-C₁₀ alkoxy group, a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₂₀ aryl group, a C₆-C₂₀ aryloxy group, a C₆-C₂₀ arylthio group, a C₂-C₂₀ heteroaryl group.

In an embodiment of formula (I), R⁴ is a phenyl group, a pentalenyl group, an indenyl group, a naphthyl group, an azulenyl group, a heptalenyl group, an indacenyl group, an acenaphthyl group, a fluorenyl group, a spiro-fluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pentacenyl group, a rubicenyl group, a coronenyl group, an ovalenyl group, a pyrrolyl group, a thiophenyl group, a furanyl group, an imidazolyl group, a pyrazolyl group, a thiazolyl group, an isothiazolyl group, an oxazolyl group, an isooxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a carbazolyl group, a phenanthridinyl group, an acridinyl group, a phenanthrolinyl group, a phenazinyl group, a benzoimidazolyl group, a benzofuranyl group, a benzothiophenyl group, an isobenzothiazolyl group, a benzooxazolyl group, an isobenzooxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, and an imidazopyrimidinyl group;

a phenyl group, a pentalenyl group, an indenyl group, a naphthyl group, an azulenyl group, a heptalenyl group, an indacenyl group, an acenaphthyl group, a fluorenyl group, a spiro-fluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pentacenyl group, a rubicenyl group, a coronenyl group, an ovalenyl group, a pyrrolyl group, a thiophenyl group, a furanyl group, an imidazolyl group, a pyrazolyl group, a thiazolyl group, an isothiazolyl group, an oxazolyl group, an isooxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a carbazolyl group, phenanthridinyl, acridinyl, phenanthrolinyl, phenazinyl, a benzoimidazolyl group, a benzofuranyl group, a benzothiophenyl group, an isobenzothiazolyl group, a benzooxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, and an imidazopyrimidinyl group, each substituted with at least one selected from —F, —Cl, —Br, —I, a hydroxyl group, a cyano group, a nitro group, a C₀-C₁₀ amino group, a C₁-C₁₀ alkyl group, a C₁-C₁₀ alkoxy group, a phenyl group, a phenyl group substituted with a C₁-C₁₀ alkyl group, a pentalenyl group, an indenyl group, a naphthyl group, an azulenyl group, a heptalenyl group, an indacenyl group, an acenaphthyl group, a fluorenyl group, a spiro-fluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl i

group, a hexacenyl group, a pentacenyl group, a rubicenyl group, a coronenyl group, an ovalenyl group, a pyrrolyl group, a thiophenyl group, a furanyl group, an imidazolyl group, a pyrazolyl group, a thiazolyl group, an isothiazolyl group, an oxazolyl group, an isooxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a carbazolyl group, a phenanthridinyl group, an acridinyl group, a phenanthrolinyl group, a phenazinyl group, a benzoimidazolyl group, a benzofuranyl group, a benzothiophenyl group, an isobenzothiazolyl group, a benzooxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, an imidazopyrimidinyl group, a biphenyl group.

In an embodiment of formula (I):

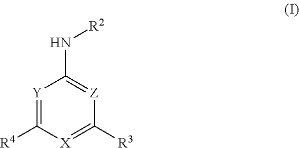

X, Y, and Z are each independently N or CH;

R² is selected from the group consisting of —(CH₂)ₙ—NR⁵R⁶, —(CH₂),C(O)—NR⁵R⁶, —(CH₂)ₙO(CH₂)ₙ—NR⁵R⁶, —(CH₂),CF₂(CH₂)ₙ

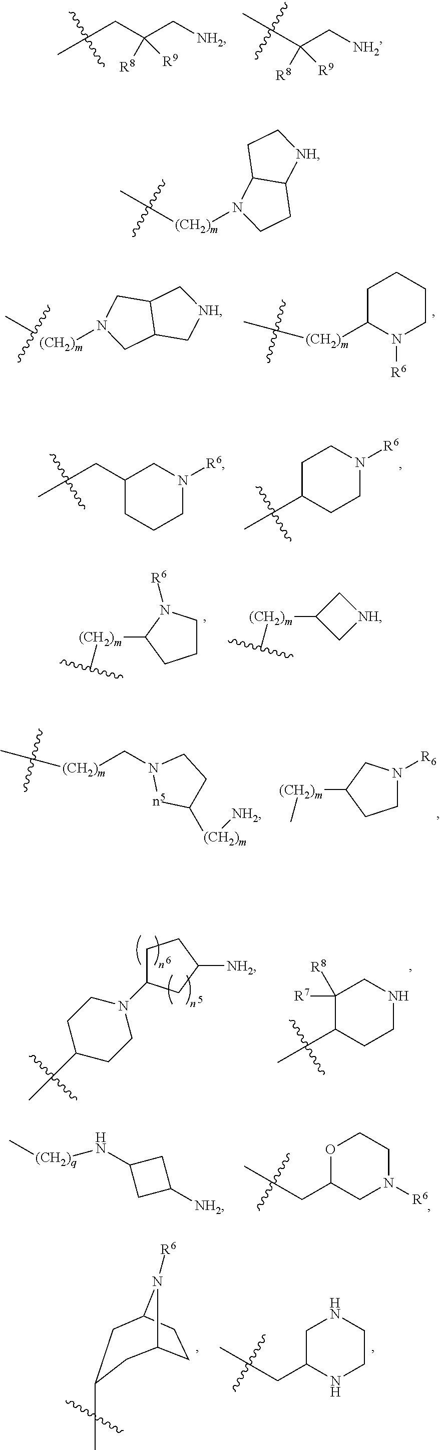

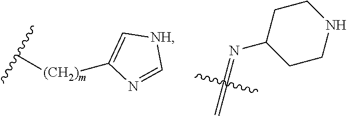

n⁵is 1 or 2;

n⁶ is 1 or 2,

q is 1 or 2,

m is 1 or 2,

R⁵ is selected from the group consisting of H, CH₃, (CH₂)NH₂, C(O)O(CH₂)ₘ,

R⁶ is selected from the group consisting of H, CH₃, (CH₂)ₙNH₂, C(O)O(CH₂)ₘ,

R⁷ is at each occurrence independently selected from the group consisting of H and F

R⁸ is at each occurrence independently selected from the group consisting of H and F

R³ is hydrogen; CF₃; a five-membered monocyclic heteroaryl ring including at least one heteroatom selected from the group consisting from O, N, and S that is optionally substituted with up to 2 groups selected from C₁₋₄ linear alkyl, C₃₋₄ branched alkyl, C₁₋₄ linear alkoxy, C₃₋₄ branched alkoxy, CF₃, CF₃O, halogen

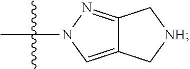 [SMILES: *n1cc2c(n1)CNC2]

a five-membered monocyclic heteroaryl ring including at least one heteroatom selected from the group consisting from O, N, and S; a phenyl ring that is optionally substituted with up to 2 groups selected from C₁₋₄ linear alkyl, C₃₋₄ branched alkyl, C₁₋₄ linear alkoxy, C₃₋₄ branched alkoxy, C₃₋₆ cycloalkyl, C₃₋₆ cycloalkoxy, CF₃, CF₃O, halogen, NR^{3a}R^{3b}, SO₂NHR^{3a},

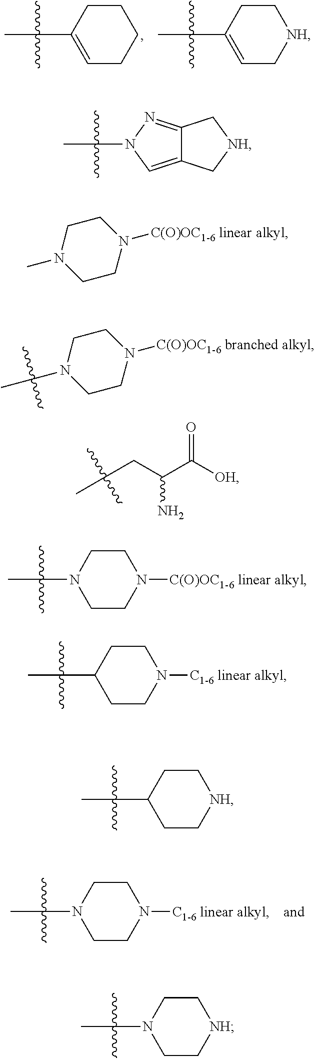

a pyridine ring that is optionally substituted with up to two groups selected from

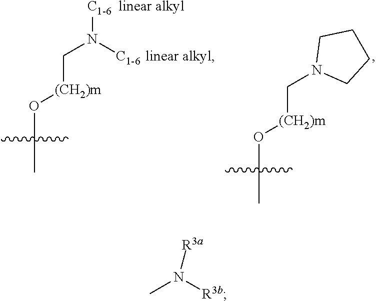

R^{3a} is at each occurrence independently selected from the group consisting of C₁₋₄ linear alkyl and C₃₋₄ branched alkyl;

R^{3b} is at each occurrence independently selected from the group consisting of C₁₋₄ linear alkyl and C₃₋₄ branched alkyl;

m is 1 or 2;

R⁴ is hydrogen; a phenyl ring optionally substituted with up to two groups selected from, F, linear alkoxy, branched alkoxy, CF₃O,

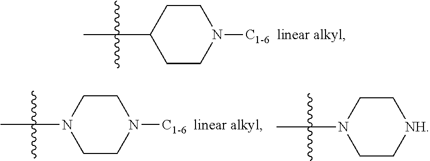

The compounds of the present invention include a compound having formula (II):

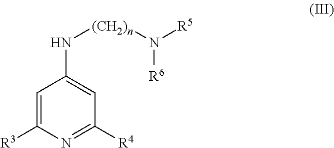

including an enantiomer, a diastereomer, a hydrate, a solvate, a pharmaceutically acceptable salt, an isotopic analog, a prodrug, and a complex thereof.

The compounds of the present invention include a compound having formula (III):

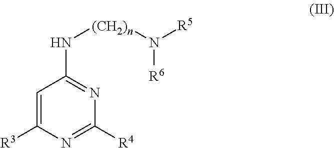

including an enantiomer, a diastereomer, a hydrate, a solvate, a pharmaceutically acceptable salt, an isotopic analog, a prodrug, and a complex thereof.

The compounds of the present invention include a compound having formula (IV):

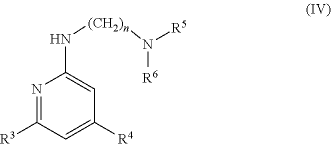

including an enantiomer, a diastereomer, a hydrate, a solvate, a pharmaceutically acceptable salt, an isotopic analog, a prodrug, and a complex thereof.

The compounds of the present invention include a compound having formula (V):

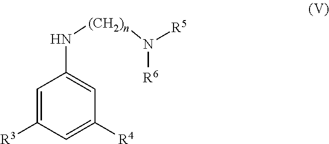

including an enantiomer, a diastereomer, a hydrate, a solvate, a pharmaceutically acceptable salt, an isotopic analog, a prodrug, and a complex thereof.

The compounds of the present invention include a compound having formula (VI):

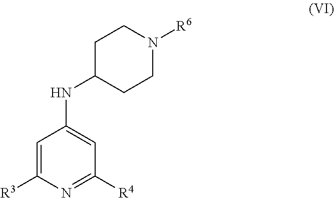

including an enantiomer, a diastereomer, a hydrate, a solvate, a pharmaceutically acceptable salt, an isotopic analog, a prodrug, and a complex thereof.

The compounds of the present invention include a compound having formula (VII):

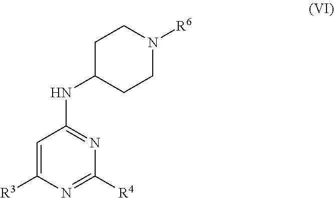

including an enantiomer, a diastereomer, a hydrate, a solvate, a pharmaceutically acceptable salt, an isotopic analog, a prodrug, and a complex thereof.

The compounds of the present invention include a compound having formula (VIII):

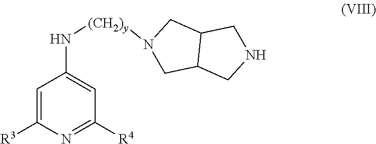

including an enantiomer, a diastereomer, a hydrate, a solvate, a pharmaceutically acceptable salt, an isotopic analog, a prodrug, and a complex thereof.

The compounds of the present invention include a compound having formula (IX):

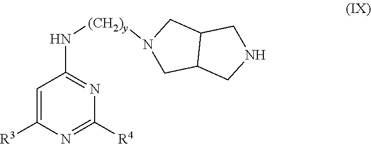

including an enantiomer, a diastereomer, a hydrate, a solvate, a pharmaceutically acceptable salt, an isotopic analog, a prodrug, and a complex thereof.

The compounds of the present invention include a compound having formula (X):

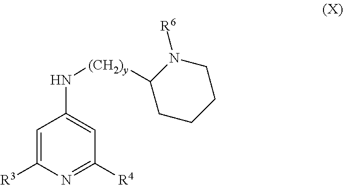

including an enantiomer, a diastereomer, a hydrate, a solvate, a pharmaceutically acceptable salt, an isotopic analog, a prodrug, and a complex thereof. Wherein R⁶ is selected from the group consisting of H, CH₃, (CH₂)ₙNH₂,

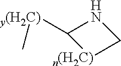

The compounds of the present invention include a compound having formula (XI):

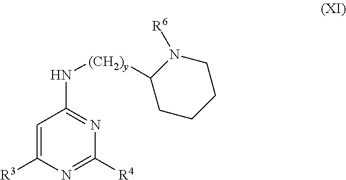

including an enantiomer, a diastereomer, a hydrate, a solvate, a pharmaceutically acceptable salt, an isotopic analog, a prodrugs, and a complex thereof. Wherein R⁶ is selected from the group consisting of H, CH₃, (CH₂)ₙNH₂,

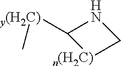

The compounds of the present invention include a compound having formula (XII):

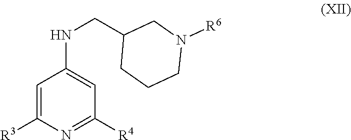

including an enantiomer, a diastereomer, a hydrate, a solvate, a pharmaceutically acceptable salt, an isotopic analog, a prodrug, and a complex thereof. Wherein R⁶ is selected from the group consisting of H, CH₃, (CH₂)ₙNH₂,

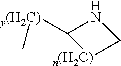

The compounds of the present invention include a compound having formula (XIII):

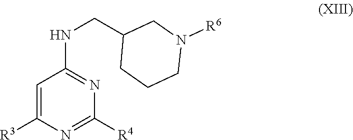

including an enantiomer, a diastereomer, a hydrate, a solvate, a pharmaceutically acceptable salt, an isotopic analog, a prodrug, and a complex thereof. Wherein R⁶ is selected from

the group consisting of H, CH₃, (CH₂)ₙNH₂,

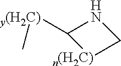

The compounds of the present invention include a compound having formula (XIV):

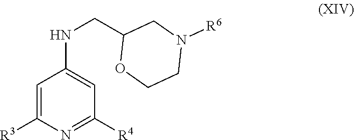

including an enantiomer, a diastereomer, a hydrate, a solvate, a pharmaceutically acceptable salt, an isotopic analog, a prodrug, and a complex thereof. Wherein R⁶ is selected from the group consisting of H, CH₃, (CH₂)ₙNH₂,

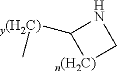

The compounds of the present invention include a compound having formula (XV):

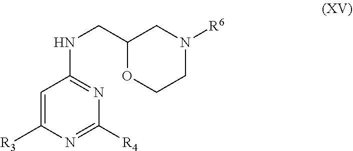

including an enantiomer, a diastereomer, a hydrate, a solvate, a pharmaceutically acceptable salt, an isotopic analog, a prodrug, and a complex thereof. Wherein R⁶ is selected from the group consisting of H, CH₃, (CH₂)ₙNH₂,

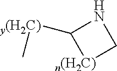

The compounds of the present invention include a compound having formula (XVI):

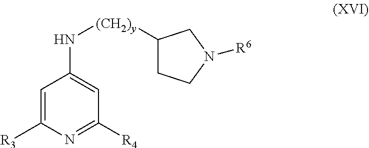

including an enantiomer, a diastereomer, a hydrate, a solvate, a pharmaceutically acceptable salt, an isotopic analog, a prodrug, and a complex thereof. Wherein R⁶ is selected from the group consisting of H, CH₃, (CH₂)ₙNH₂,

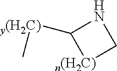

The compounds of the present invention include a compound having formula (XVII):

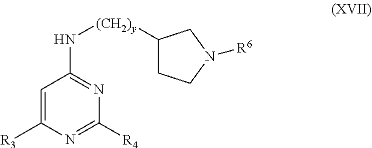

including an enantiomer, a diastereomer, a hydrate, a solvate, a pharmaceutically acceptable salt, an isotopic analog, a prodrug, and a complex thereof. Wherein R⁶ is selected from the group consisting of H, CH₃, (CH₂)ₙNH₂,

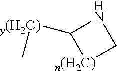

The compounds of the present invention include a compound having formula (XVIII):

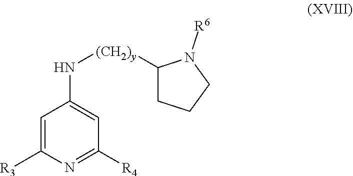

including an enantiomer, a diastereomer, a hydrate, a solvate, a pharmaceutically acceptable salt, an isotopic analog, a prodrug, and a complex thereof.

In some embodiments, a compound having formula (XVIII), Wherein R⁶ is selected from the group consisting of H, CH₃, (CH₂)ₙNH₂,

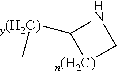

The compounds of the present invention include a compound having formula XIX):

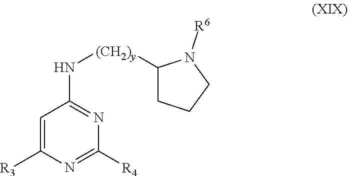

including an enantiomer, a diastereomer, a hydrate, a solvate, a pharmaceutically acceptable salt, an isotopic analog, a prodrug, and a complex thereof. In some embodiments, a compound having formula (XIX), Wherein R⁶ is selected from the group consisting of H, CH₃, (CH₂)ₙNH₂,

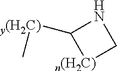

The compounds of the present invention include a compound having formula (XX):

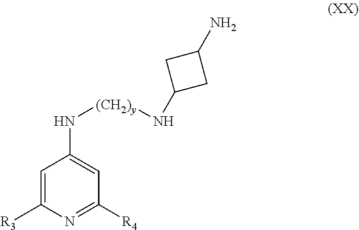

including an enantiomer, a diastereomer, a hydrate, a solvate, a pharmaceutically acceptable salt, an isotopic analog, a prodrug, and a complex thereof.

The compounds of the present invention include a compound having formula (XXI):

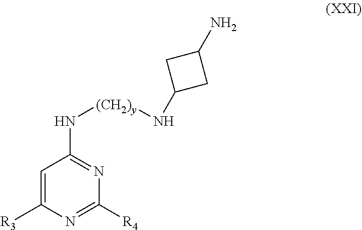

including an enantiomers, a diastereomer, a hydrate, a solvate, a pharmaceutically acceptable salt, an isotopic analog, a prodrug, and a complex thereof.

The compounds of the present invention include a compound having formula (XXII):

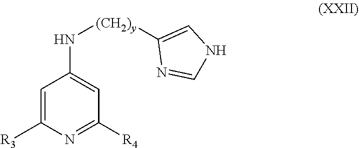

including an enantiomer, a diastereomer, a hydrate, a solvate, a pharmaceutically acceptable salt, an isotopic analog, a prodrug, and a complex thereof.

The compounds of the present invention include a compound having formula (XXIII):

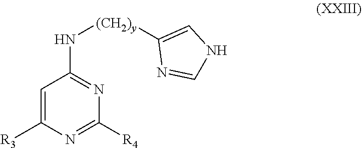

including an enantiomer, a diastereomer, a hydrate, a solvate, a pharmaceutically acceptable salt, an isotopic analog, a prodrug, and a complex thereof.

The compounds of the present invention include a compound having formula (XXIV):

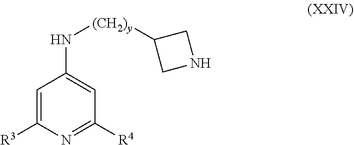

including an enantiomer, a diastereomer, a hydrate, a solvate, a pharmaceutically acceptable salt, an isotopic analog, a prodrug, and a complex thereof.

The compounds of the present invention include a compound having formula (XXV):

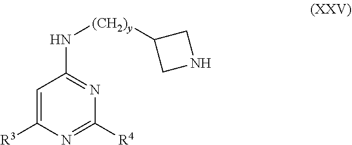

including an enantiomer, a diastereomer, a hydrate, a solvate, a pharmaceutically acceptable salt, an isotopic analog, a prodrug, and a complex thereof.

The compounds of the present invention include a compound having formula (XXVI):

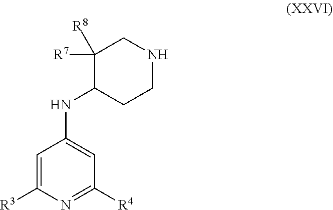

including an enantiomer, a diastereomer, a hydrate, a solvate, a pharmaceutically acceptable salt, an isotopic analog, a prodrug, and a complex thereof. Wherein R⁷ and R⁸ at each occurrence independently is H and F.

The compounds of the present invention include a compound having formula (XXVII):

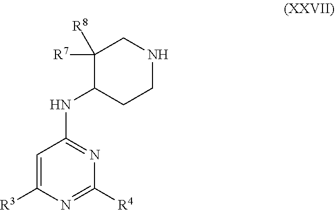

including an enantiomer, a diastereomer, a hydrate, a solvate, a pharmaceutically acceptable salt, an isotopic analog, a prodrug, and a complex thereof. Wherein R⁷ and R⁸ at each occurrence independently is H and F.

In another embodiment, disclosed is a method for treating or preventing a disease that involves TDP-43, the method including administering to a subject an effective amount of at least one compound having formula (II):

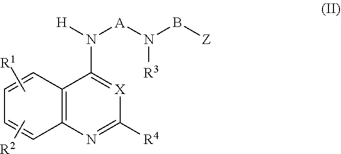

an enantiomer, a diastereomer, a hydrate, a solvate, a pharmaceutically acceptable salt, an isotopic analog, a prodrug or a complex thereof,

wherein, in formula (II):

X is N or CH;

R¹ and R² are the same or different and are each independently H, halogen, C₁-C₄ alkyl, C₁-C₄ alkoxy, CF₃, or CF₃O, wherein C₁-C₄ alkyl and C₁-C₄ alkoxy are optionally substituted with at least one fluorine;

R³ is H, C₁-C₇ alkyl, C₄-C₇ carbocyclic, aryl, heteroaryl, heterocyclic, heterocyclic alkyl, wherein R³ is optionally taken together with either A or B to form a ring consisting of 4-7 ring members;

R⁴=phenyl or phenyl substituted with C₁-C₄ alkyl, C₁-C₄ alkoxy, CF₃, CF₃O, halogen, amino, or sulfonamide;

A=(CH₂)ₙ, wherein n is 2-4, wherein A and R³ are optionally taken together to form a ring consisting of 4-7 ring members;

B=(CH₂)ₙ, wherein n is 2-6, wherein B and R³ are optionally taken together to form a ring consisting of 4-7 members, and wherein B is optionally substituted with one or more substituents selected from C₁-C₄ alkyl, hydroxy, C₁-C₄ alkoxy, CF₃, and CF₃O; and

Z is OH, C₁-C₄ alkoxy, OCF₃, or N with one or more of H, C₁-C₄ alkyl, heteroaryl, substituted heteroaryl, C₁-C₄ sulfonamido, substituted amido.

For example, the compound having formula (II) may be represented by the following formula((II-1)

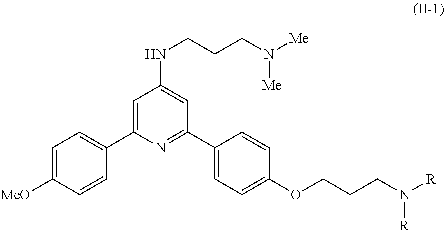

wherein, in formula (III), each R is hydrogen or C₁-C₄ alkyl, wherein groups R are optionally connected to form a ring.

In another embodiment, disclosed is a method for treating or preventing a disease that involves TDP-43, the method including administering to a subject an effective amount of at least one compound having formulae (IIIa) or (IIIb):

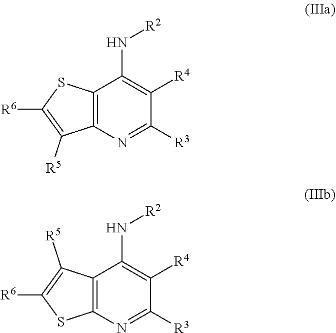

an enantiomer, a diastereomer, a hydrate, a solvate, a pharmaceutically acceptable salt, an isotopic analog, a prodrug or a complex thereof,

wherein, in formulae (IIa) and (IIIb):

R² is a substituted or unsubstituted C₁-C₂₀ linear, branched, or cyclic organic group including at least one nitrogen; and

R³, R⁴, R⁵, and R⁶ are each independently hydrogen, halogen, a hydroxyl group, a cyano group, a nitro group, a substituted or unsubstituted C₀-C₁₀ amino group, a substituted or unsubstituted C₁-C₁₀ alkyl group, a substituted or unsubstituted C₂-C₁₀ alkenyl group, a substituted or unsubstituted C₂-C₁₀ alkynyl group, and a substituted or unsubstituted C₁-C₁₀ alkoxy group, a substituted or unsubstituted C₃-C₁₀ cycloalkyl group, a substituted or unsubstituted C₂-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₂₀ aryl group, a substituted or unsubstituted C₆-C₂₀ aryloxy group, a substituted or unsubstituted C₆-C₂₀ arylthio group, a substituted or unsubstituted C₂-C₂₀ heteroaryl group, a substituted or unsubstituted C₂-C₂₀ heteroaryloxy group, or a substituted or unsubstituted C₂-C₂₀ heteroarylthio group.

In another embodiment, disclosed is a method for treating or preventing a disease that involves TDP-43, the method including administering to a subject an effective amount of at least one compound having formula (IV):

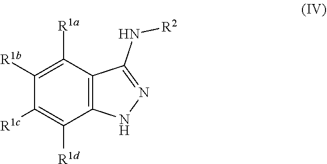

an enantiomer, a diastereomer, a hydrate, a solvate, a pharmaceutically acceptable salt, an isotopic analog, a prodrug or a complex thereof,

wherein, in formula (IV):

R² is a substituted or unsubstituted C₁-C₂₀ linear, branched, or cyclic organic group including at least one nitrogen; and

R^{1a}, R^{1l}, R^{1c}, and Rid are each independently hydrogen, halogen, a hydroxyl group, a cyano group, a nitro group, a substituted or unsubstituted C₀-C₁₀ amino group, a substituted or unsubstituted C₁-C₁₀ alkyl group, a substituted or unsubstituted C₂-C₁₀ alkenyl group, a substituted or unsubstituted C₂-C₁₀ alkynyl group, and a substituted or unsubstituted C₁-C₁₀ alkoxy group, a substituted or unsubstituted C₃-C₁₀ cycloalkyl group, a substituted or unsubstituted C₂-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₂₀ aryl group, a substituted or unsubstituted C₆-C₂₀ aryloxy group, a substituted or unsubstituted C₆-C₂₀ arylthio group, a substituted or unsubstituted C₂-C₂₀ heteroaryl group, a substituted or unsubstituted C₂-C₂₀ heteroaryloxy group, or a substituted or unsubstituted C₂-C₂₀ heteroarylthio group.

In another embodiment, disclosed is a method for treating or preventing a disease that involves TDP-43, the method including administering to a subject an effective amount of at least one compound having formula (V):

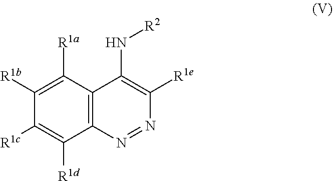

an enantiomer, a diastereomer, a hydrate, a solvate, a pharmaceutically acceptable salt, an isotopic analog, a prodrug or a complex thereof,

wherein, in formula (IV):

R² is a substituted or unsubstituted C₁-C₂₀ linear, branched, or cyclic organic group including at least one nitrogen; and

R^{1a}, R^{1b}, R^{1c}, R^{1d}, and R^{1c} are each independently hydrogen, halogen, a hydroxyl group, a cyano group, a nitro group, a substituted or unsubstituted C₀-C₁₀ amino group, a substituted or unsubstituted C₁-C₁₀ alkyl group, a substituted or unsubstituted C₂-C₁₀ alkenyl group, a substituted or unsubstituted C₂-C₁₀ alkynyl group, and a substituted or unsubstituted C₁-C₁₀ alkoxy group, a substituted or unsubstituted C₃-C₁₀ cycloalkyl group, a substituted or unsubstituted C₂-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₂₀ aryl group, a substituted or unsubstituted C₆-C₂₀ aryloxy group, a substituted or unsubstituted C₆-C₂₀ arylthio group, a substituted or unsubstituted C₂-C₂₀ heteroaryl group, a substituted or unsubstituted C₂-C₂₀ heteroaryloxy group, or a substituted or unsubstituted C₂-C₂₀ heteroarylthio group.

For example, the compound having formula (V) may be represented by the following formula (V-1):

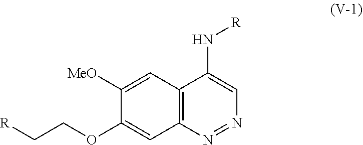

wherein, in Formula (V-1), each R is independently a substituted or unsubstituted C₁-C₂₀ linear, branched, or cyclic organic group including at least one nitrogen.

### Process of Preparation

The present invention further relates to a process for preparing the TDP-43 binding agents of the present invention.

Compounds of the present teachings can be prepared in accordance with the procedures outlined herein, from commercially available starting materials, compounds known in the literature, or readily prepared intermediates, by employing standard synthetic methods and procedures known to those skilled in the art. Standard synthetic methods and procedures for the preparation of organic molecules and functional group transformations and manipulations can be readily obtained from the relevant scientific literature or from standard textbooks in the field. It will be appreciated that where typical or preferred process conditions (i.e., reaction temperatures, times, mole ratios of reactants, solvents, pressures, etc.) are given, other process conditions can also be used unless otherwise stated. Optimum reaction conditions can vary with the particular reactants or solvent used, but such conditions can be determined by one skilled in the art by routine optimization procedures. Those skilled in the art of organic synthesis will recognize that the nature and order of the synthetic steps presented can be varied for the purpose of optimizing the formation of the compounds described herein.

The processes described herein can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry, or by chromatography such as high pressure liquid chromatography (HPLC), gas chromatography (GC), gel-permeation chromatography (GPC), or thin layer chromatography (TLC).

Preparation of the compounds can involve protection and deprotection of various chemical groups. The need for protection and deprotection and the selection of appropriate protecting groups can be readily determined by one skilled in the art. The chemistry of protecting groups can be found, for example, in Greene et al., Protective Groups in Organic Synthesis, 2d. Ed. (Wiley & Sons, 1991), the entire disclosure of which is incorporated by reference herein for all purposes.

The reactions or the processes described herein can be carried out in suitable solvents which can be readily selected by one skilled in the art of organic synthesis. Suitable solvents typically are substantially nonreactive with the reactants, intermediates, and/or products at the temperatures at which the reactions are carried out, i.e., temperatures that can range from the solvent's freezing temperature to the solvent's boiling temperature. A given reaction can be carried out in one solvent or a mixture of more than one solvent. Depending on the particular reaction step, suitable solvents for a particular reaction step can be selected.

The compounds of these teachings can be prepared by methods known in the art of organic chemistry. The reagents used in the preparation of the compounds of these teachings can be either commercially obtained or can be prepared by standard procedures described in the literature. For example, compounds of the present invention can be prepared according to the method illustrated in the General Synthetic Schemes:

### General Synthetic Schemes for Preparation of Compounds

The reagents used in the preparation of the compounds of this invention can be either commercially obtained or can be prepared by standard procedures described in the literature. In accordance with this invention, compounds in the genus may be produced by one of the following reaction schemes.

Compounds of formula (I) may be prepared according to the process outlined in Schemes 1-4.

One method to prepare compounds of the invention involves the coupling of a 2,4,6 trichloro pyridine, 2,6 dibromo-4-chloro pyridine, pyrimidine, triazine or benzene in the presence of Pd(pddf)Cl₂, Pd(PPh₃)₄ or the like and TBAF in in dioxane, ethanol, toluene with or without water with arylboronic acid, followed by a second coupling in the presence of Pd(pddf)Cl₂ Pd(PPh₃)₄ or the like and TBAF in dioxane, ethanol, toluene with or without water with arylboronic acid or alkenylboronic acid, followed by a coupling reaction in the presence of Pd₂dba₃, Pd(OAc)₂, X-phos, Dave-Phos, NaOtBu, potassium carbonate, in t-BuOH, dioxane, water, alternatively followed by a reductive amination in the presence of Na(OAc)₃ in acetic acid and 1,2-dichloreethane. (Schemes 1 and 2):

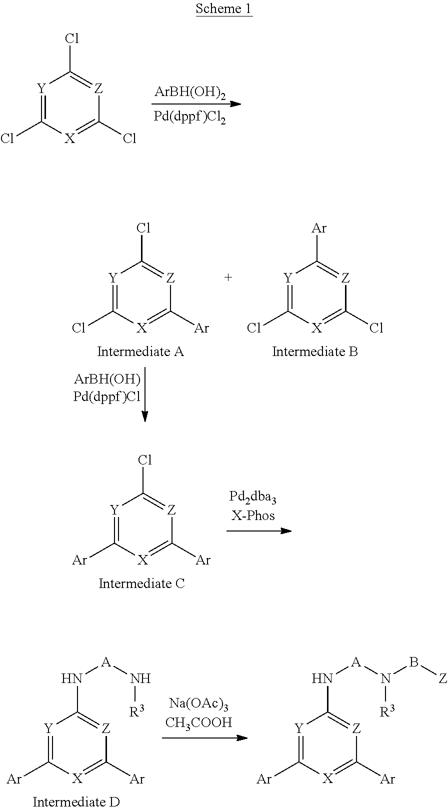

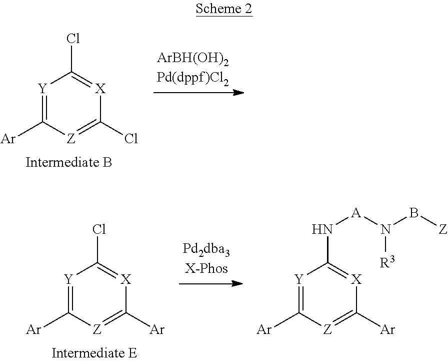

Another method to prepare compounds of the invention involves the coupling of a 2,4-dichloro-6-aryl pyridine, pyrimidine, triazine or benzene in the presence of Pd(PPh₃)₄ and K₂CO₃ in Dioxane water (2:1) with alkeneboronic acid, followed by reaction with a primary amine in the presence of Pd₂dba₃, X-Phos, NaOtBu in t-Butanol and subsequent reduction with Pd on Carbon in methanol under H₂ atmosphere. (Scheme 3).

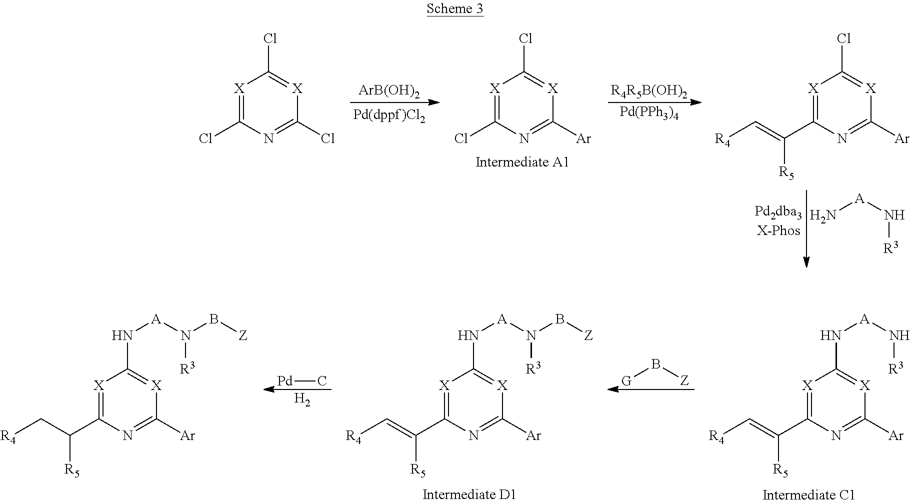

Another method to prepare compounds of the invention involves the coupling of a 2,4-dichloro-6-aryl pyridine, pyrimidine, triazine or benzene using Pd(PPh₃)₂Cl₂ and CuI in TEA/THF 1:2 with an alkyne using standard Sonogashira coupling conditions followed by reaction with a primary amine primary in the presence of Pd₂dba₃, X-Phos or BINAP, NaOtBu in t-Butanol or Dioxane (Scheme 4). R₄ is alkyl, alkyl amine, alternatively further substituted.

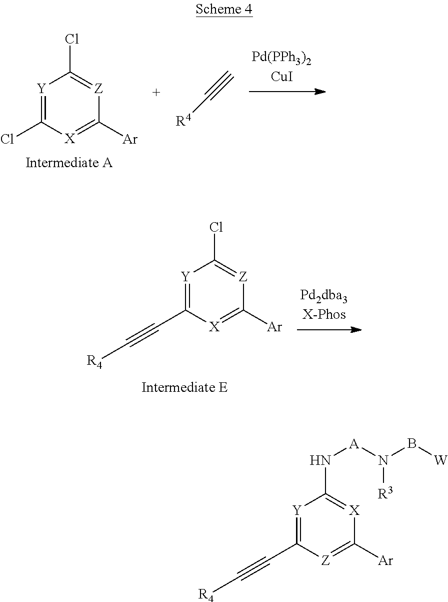

Another method (Scheme 5) to prepare the compounds of the invention involves the coupling of and aryl boronic acid or ester to e.g. 2,4 dichlorothienopyridine, in the presence of Pd(PPh₃)₄ and K₂CO₃ in dioxane-water (2:1), followed by reaction with a primary amine in the presence of Pd₂dba₃, X-Phos, NaOtBu in t-butanol, deprotection of the amine as needed using HCl in dioxane.

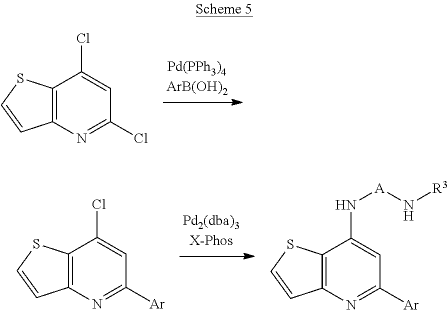

It is to be understood that further homologation of pendant substitution can provide additional compounds that fall within the scope of the invention. The use and deprotection of amine functional groups can be a useful way to further produce relevant structural variation. For example, amine functionality can be converted to sulfonamide or amide groups.

The invention is further illustrated by the following non-limiting examples.

Alpha-screen assay. Displacement of bt-TG6 binding was measured using Alpha Screen technology as previously described (Cassel, J. A. et al., Journal of Biomolecular Screening, 2010, 15, 1099-1106; Cassel, J. A.; Reitz, A. B. et al. The effects of small molecule inhibitors of nucleic acid binding to TDP-43 on TDP-43 metabolism and function. Biochimie, 2012, 94, 1974-1981). Briefly, assays contained TDP-43 1-260 (15 ng/mL, 0.27 nM), 10 mg/mL of both streptavidin donor beads and anti-GST acceptor beads with various concentrations of bt-TAR32 (˜18 to +14 in the HIV1 LTR) and test compound in a final volume of 20 mL of 25 mM Tris, 0.1% CHAPS, pH 7.4 in white, opaque low volume 384-well plates. After 3 h incubation at room temperature, the AlphaScreen signal was measured using a Synergy 2 plate reader (Biotek, Winooski, VT). For IC₅₀ determinations, the concentration of bt-TAR32 was 0.125 nM and for inhibitor characterization experiments, the concentrations of test compounds ranged from 50 to 0.02 μM in 8 half log dilutions. Nonspecific binding was determined in the presence of 10 mM bt-TDP43 and was less than 10% of total binding. IC₅₀ values were obtained from nonlinear regression fits of the data to a one-site binding model using Graph Pad Prism, version 5.0 (GraphPad Software, San Diego, CA). Comparison of competitive inhibition and mixed type inhibition models were evaluated using the F-test on global fits of the data also in Graph Pad Prism, version 5.0.

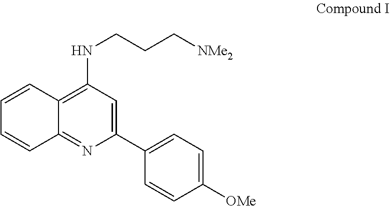 [SMILES: COc1ccc(-c2cc(NCCCN(C)C)c3ccccc3n2)cc1]

We have previously reported the activity of Compound I, listed as 1 in Biochimie, 2012, 94, 1974-1981, as being active in a TDP-43 alpha-screen, however, using bt-(TG)₆ and not bt-TAR32.

In addition, Compound I promoted caspase-7 cleavage of TDP-43. However, we have unexpectedly found a significant boost in activity when we append to the terminal amino group further substitution of either an aminoalkyl or alkoxyalkyl. For example, Example 3 exhibits an increase in potency of 11-18X (14X average) in the same alpha-screen assay using bt-TAR32 when the two compounds are compared side-by-side in three independent assays.

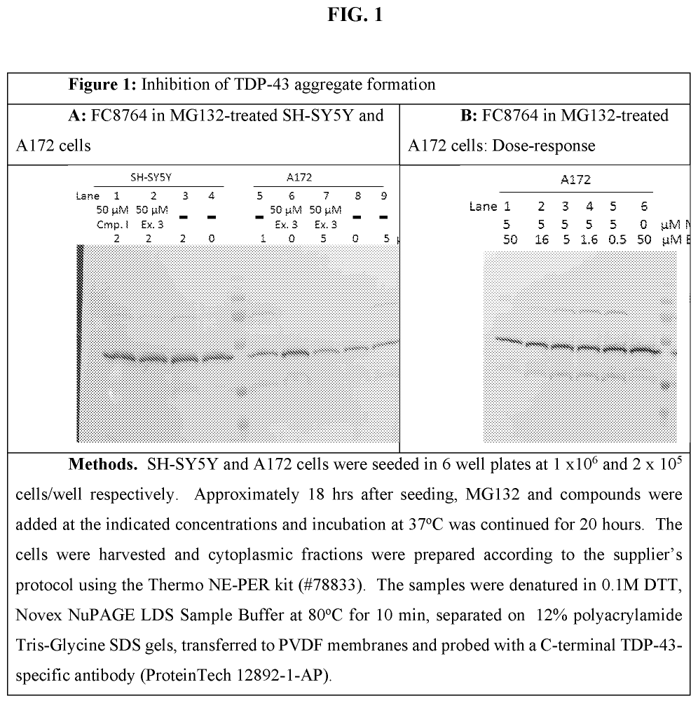

It has been reported (Eersel et al. PLoS ONE, 2011, 6, e22850) that inhibition of proteasome activity in neuronal cells results in accumulation and aggregation of TDP-43 in the cytoplasm that recapitulates major pathological features of neurodegenerative disease including amyotrophic lateral sclerosis (ALS), frontotemporal lobar degeneration (FTLD) and Alzheimer's disease (AD). To investigate the consequences of inhibition of nucleic acid binding by TDP-43 on these disease-related pathologies, human neuroblastoma (SH-SY5Y) and glioblastoma (A172) cells were treated with the proteasome inhibitor benzyloxycarbonyl-1-leucyl-1-leucyl-1-leucinal (MG-132) in the presence and absence of Example 3 (FIG. 1). Cytoplasmic protein extracts were prepared after a 20 hour treatment period and resolved on a denaturing gel to identify TDP-43 immuno-reactive products by Western analysis. In FIG. 1A, full-length 43 kD TDP-43 species was evident in all samples and treatment with MG-132 resulted in the appearance of a higher MW 70 kD band indicative of aggregation (lane 3 for SH-SY5Y cells and lanes 5 and 9 for A172 cells). Importantly, the 70 kD reactive product was absent in both cell types co-treated with MG-132 and Example 3 (lane 2 for SH-SY5Y cells and lane 7 for A172 cells). There was only a smaller impact on levels of the 70 kD product when SH-SY5Y cells were co-treated with MG132 and Compound I (lane 1). A dose response study in A172 cells (FIG. 1B) showed a progressive decline in levels of the 70 kD band relative to full-length TDP-43 as concentrations of Example 3 were increased from 0.5 to 50 μM. Together, these results indicate that inhibition of nucleic acid binding reduces levels of TDP-43 aggregation that is associated with and implicated in mediating neuronal pathologies of several neurodegenerative diseases.

TABLE 1
Activity of test compounds in the TDP-43-DNA alpha-screen assay.

| Example | | Activity |
|---|---|---|
| number | Structure | (μM) |
| 1 | 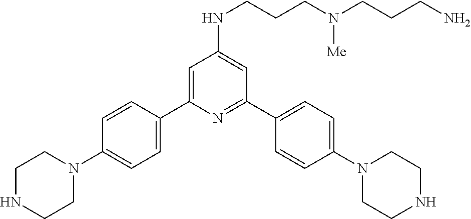 [SMILES: CN(CCCN)CCCNc1cc(-c2ccc(N3CCNCC3)cc2)nc(-c2ccc(N3CCNCC3)cc2)c1] | 0.02 |
| 2 | 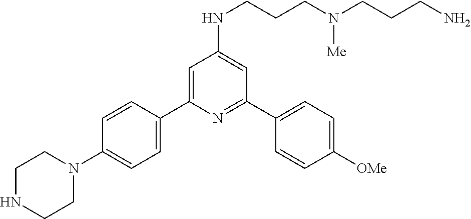 [SMILES: COc1ccc(-c2cc(NCCCN(C)CCCN)cc(-c3ccc(N4CCNCC4)cc3)n2)cc1] | 0.03 |
| 3 | 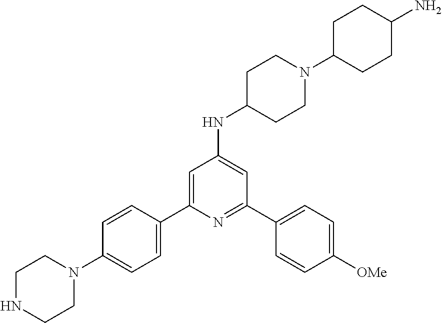 [SMILES: COc1ccc(-c2cc(NC3CCN(C4CCC(N)CC4)CC3)cc(-c3ccc(N4CCNCC4)cc3)n2)cc1] | 0.03 |
| 4 | 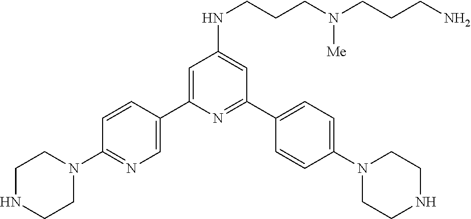 [SMILES: CN(CCCN)CCCNc1cc(-c2ccc(N3CCNCC3)cc2)nc(-c2ccc(N3CCNCC3)nc2)c1] | 0.04 |
| 5 | 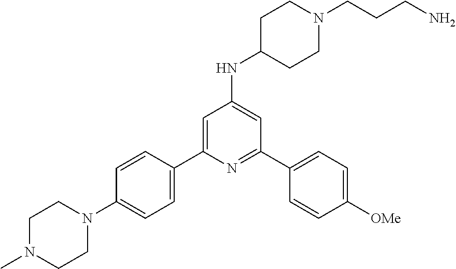 [SMILES: COc1ccc(-c2cc(NC3CCN(CCCN)CC3)cc(-c3ccc(N4CCN(C)CC4)cc3)n2)cc1] | 0.04 |
| 6 | 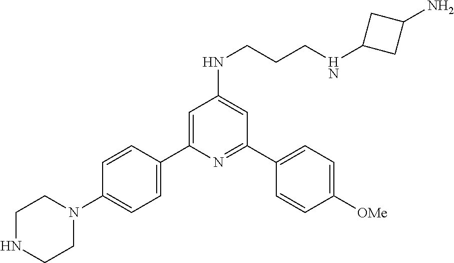 [SMILES: COc1ccc(-c2cc(NCCCNC3CC(N)C3)cc(-c3ccc(N4CCNCC4)cc3)n2)cc1] | 0.04 |
| 7 | 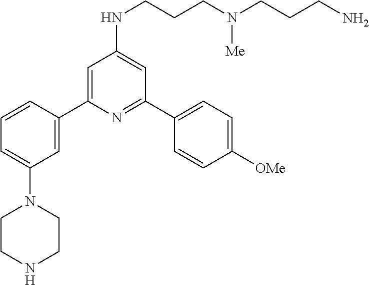 [SMILES: COc1ccc(-c2cc(NCCCN(C)CCCN)cc(-c3cccc(N4CCNCC4)c3)n2)cc1] | 0.05 |
| 8 | 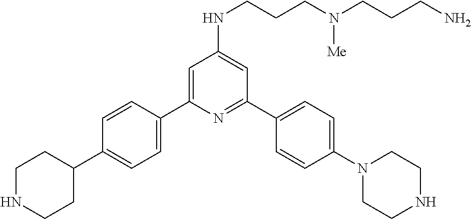 [SMILES: CN(CCCN)CCCNc1cc(-c2ccc(C3CCNCC3)cc2)nc(-c2ccc(N3CCNCC3)cc2)c1] | 0.05 |
| 9 | 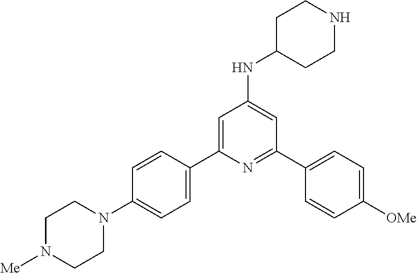 [SMILES: COc1ccc(-c2cc(NC3CCNCC3)cc(-c3ccc(N4CCN(C)CC4)cc3)n2)cc1] | 0.05 |
| 10 | 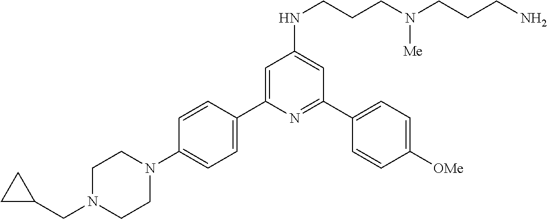 [SMILES: COc1ccc(-c2cc(NCCCN(C)CCCN)cc(-c3ccc(N4CCN(CC5CC5)CC4)cc3)n2)cc1] | 0.06 |
| 11 | 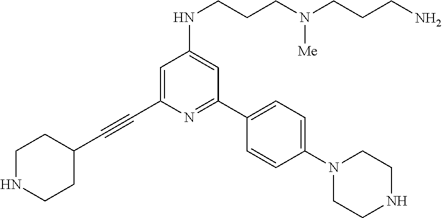 [SMILES: CN(CCCN)CCCNc1cc(C#CC2CCNCC2)nc(-c2ccc(N3CCNCC3)cc2)c1] | 0.06 |
| 12 | 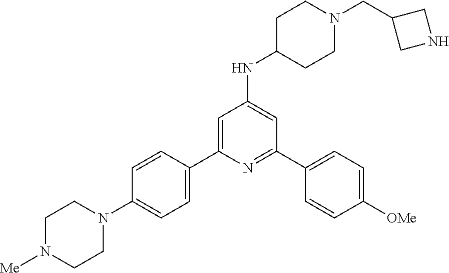 [SMILES: COc1ccc(-c2cc(NC3CCN(CC4CNC4)CC3)cc(-c3ccc(N4CCN(C)CC4)cc3)n2)cc1] | 0.06 |
| 13 | 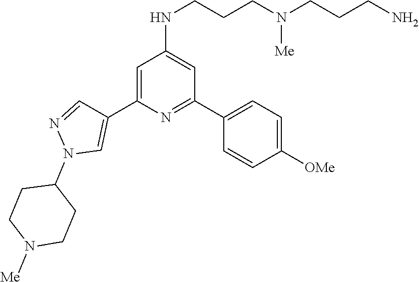 [SMILES: COc1ccc(-c2cc(NCCCN(C)CCCN)cc(-c3cnn(C4CCN(C)CC4)c3)n2)cc1] | 0.065 |
| 14 | 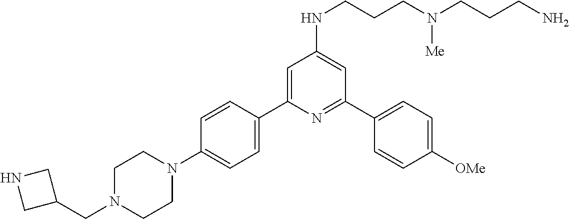 [SMILES: COc1ccc(-c2cc(NCCCN(C)CCCN)cc(-c3ccc(N4CCN(CC5CNC5)CC4)cc3)n2)cc1] | 0.07 |
| 15 | 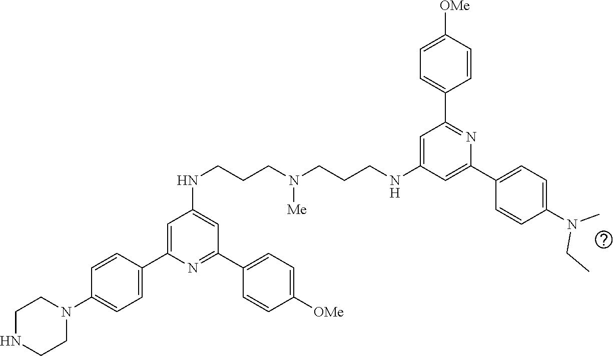 [SMILES: CCN(C)c1ccc(-c2cc(NCCCN(C)CCCNc3cc(-c4ccc(OC)cc4)nc(-c4ccc(N5CCNCC5)cc4)c3)cc(-c3ccc(OC)cc3)n2)cc1] | 0.07 |
| 16 | 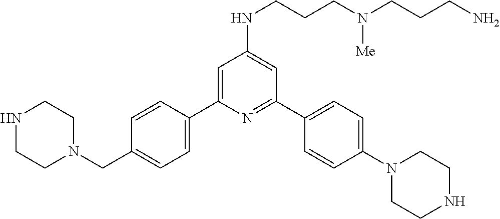 [SMILES: CN(CCCN)CCCNc1cc(-c2ccc(CN3CCNCC3)cc2)nc(-c2ccc(N3CCNCC3)cc2)c1] | 0.07 |
| 17 | 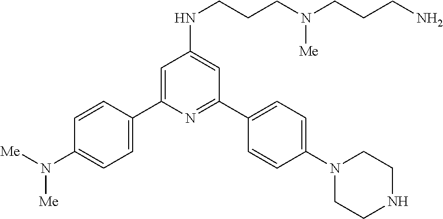 [SMILES: CN(CCCN)CCCNc1cc(-c2ccc(N(C)C)cc2)nc(-c2ccc(N3CCNCC3)cc2)c1] | 0.07 |
| 18 | 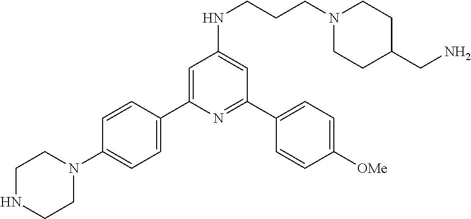 [SMILES: COc1ccc(-c2cc(NCCCN3CCC(CN)CC3)cc(-c3ccc(N4CCNCC4)cc3)n2)cc1] | 0.07 |
| 19 | 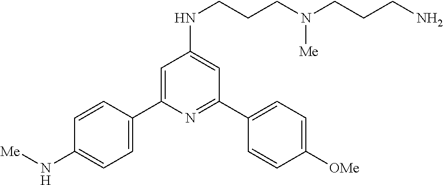 [SMILES: CNc1ccc(-c2cc(NCCCN(C)CCCN)cc(-c3ccc(OC)cc3)n2)cc1] | 0.08 |
| 20 | 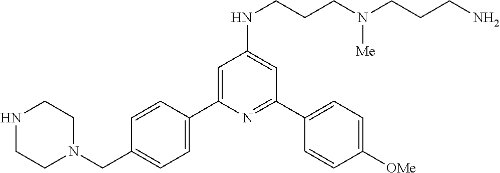 [SMILES: COc1ccc(-c2cc(NCCCN(C)CCCN)cc(-c3ccc(CN4CCNCC4)cc3)n2)cc1] | 0.08 |
| 21 | 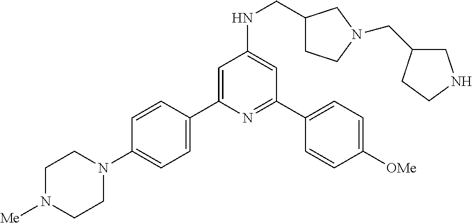 [SMILES: COc1ccc(-c2cc(NCC3CCN(CC4CCNC4)C3)cc(-c3ccc(N4CCN(C)CC4)cc3)n2)cc1] | 0.08 |
| 22 | 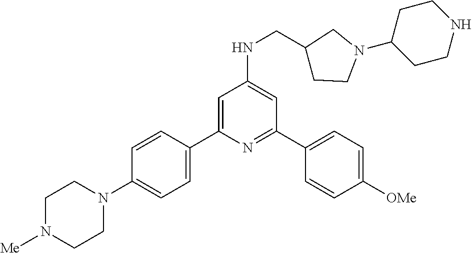 [SMILES: COc1ccc(-c2cc(NCC3CCN(C4CCNCC4)C3)cc(-c3ccc(N4CCN(C)CC4)cc3)n2)cc1] | 0.08 |
| 23 | 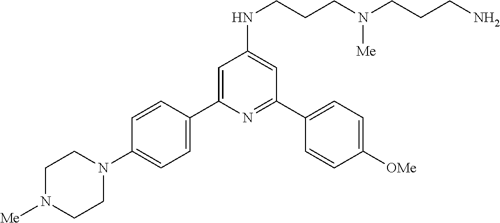 [SMILES: COc1ccc(-c2cc(NCCCN(C)CCCN)cc(-c3ccc(N4CCN(C)CC4)cc3)n2)cc1] | 0.08 |
| 24 | 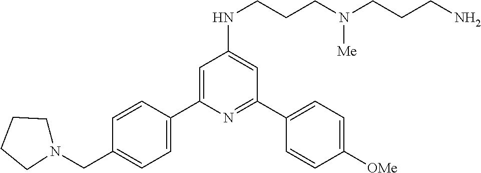 [SMILES: COc1ccc(-c2cc(NCCCN(C)CCCN)cc(-c3ccc(CN4CCCC4)cc3)n2)cc1] | 0.09 |
| 25 | 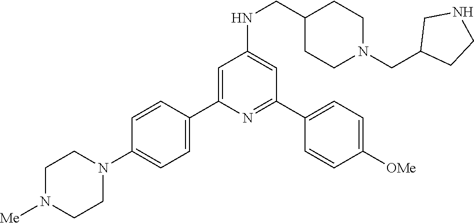 [SMILES: COc1ccc(-c2cc(NCC3CCN(CC4CCNC4)CC3)cc(-c3ccc(N4CCN(C)CC4)cc3)n2)cc1] | 0.09 |
| 26 | 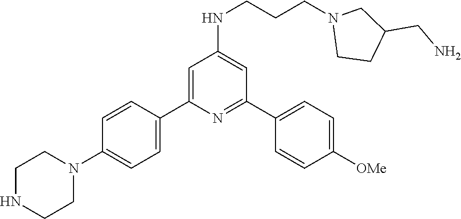 [SMILES: COc1ccc(-c2cc(NCCCN3CCC(CN)C3)cc(-c3ccc(N4CCNCC4)cc3)n2)cc1] | 0.10 |
| 27 | 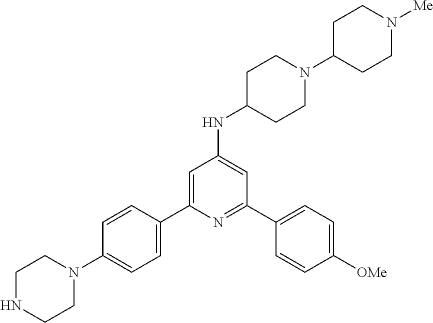 [SMILES: COc1ccc(-c2cc(NC3CCN(C4CCN(C)CC4)CC3)cc(-c3ccc(N4CCNCC4)cc3)n2)cc1] | 0.11 |
| 28 | 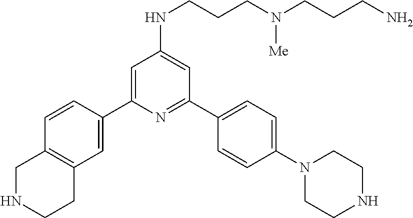 [SMILES: CN(CCCN)CCCNc1cc(-c2ccc(N3CCNCC3)cc2)nc(-c2ccc3c(c2)CCNC3)c1] | 0.11 |
| 29 | 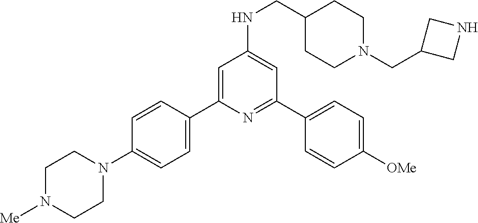 [SMILES: COc1ccc(-c2cc(NCC3CCN(CC4CNC4)CC3)cc(-c3ccc(N4CCN(C)CC4)cc3)n2)cc1] | 0.11 |
| 30 | 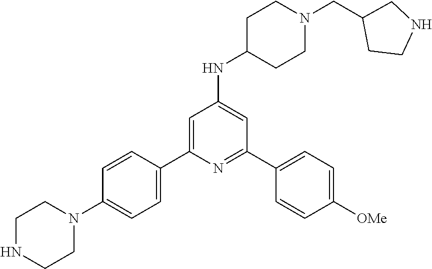 [SMILES: COc1ccc(-c2cc(NC3CCN(CC4CCNC4)CC3)cc(-c3ccc(N4CCNCC4)cc3)n2)cc1] | 0.12 |
| 31 | 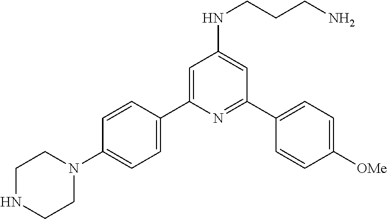 [SMILES: COc1ccc(-c2cc(NCCCN)cc(-c3ccc(N4CCNCC4)cc3)n2)cc1] | 0.12 |
| 32 | 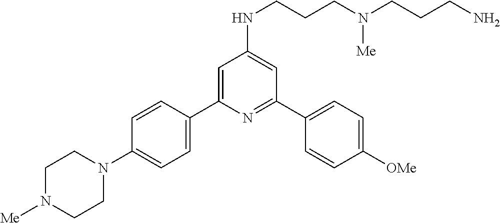 [SMILES: COc1ccc(-c2cc(NCCCN(C)CCCN)cc(-c3ccc(N4CCN(C)CC4)cc3)n2)cc1] | 0.13 |
| 33 | 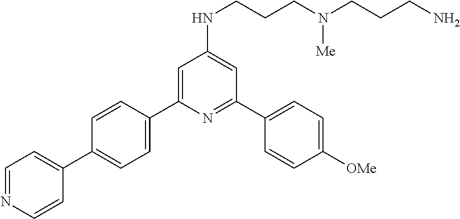 [SMILES: COc1ccc(-c2cc(NCCCN(C)CCCN)cc(-c3ccc(-c4ccncc4)cc3)n2)cc1] | 0.13 |
| 34 | 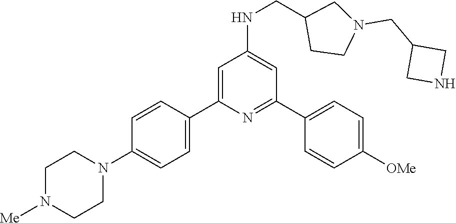 [SMILES: COc1ccc(-c2cc(NCC3CCN(CC4CNC4)C3)cc(-c3ccc(N4CCN(C)CC4)cc3)n2)cc1] | 0.14 |
| 35 | 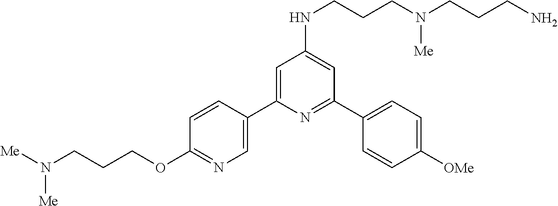 [SMILES: COc1ccc(-c2cc(NCCCN(C)CCCN)cc(-c3ccc(OCCCN(C)C)nc3)n2)cc1] | 0.15 |
| 36 | 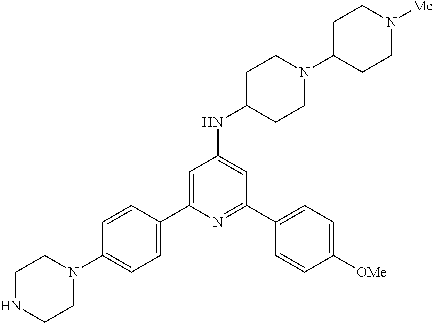 [SMILES: COc1ccc(-c2cc(NC3CCN(C4CCN(C)CC4)CC3)cc(-c3ccc(N4CCNCC4)cc3)n2)cc1] | 0.15 |
| 37 | 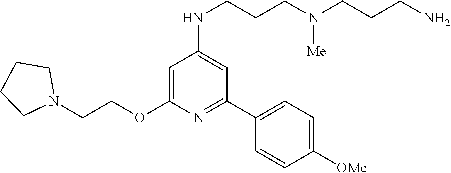 [SMILES: COc1ccc(-c2cc(NCCCN(C)CCCN)cc(OCCN3CCCC3)n2)cc1] | 0.16 |
| 38 | 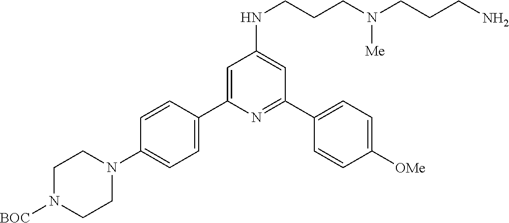 [SMILES: COc1ccc(-c2cc(NCCCN(C)CCCN)cc(-c3ccc(N4CCN(C(=O)OC(C)(C)C)CC4)cc3)n2)cc1] | 0.17 |
| 39 | 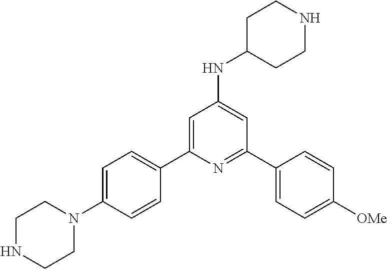 [SMILES: COc1ccc(-c2cc(NC3CCNCC3)cc(-c3ccc(N4CCNCC4)cc3)n2)cc1] | 0.19 |
| 40 | 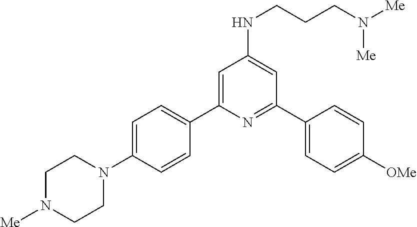 [SMILES: COc1ccc(-c2cc(NCCCN(C)C)cc(-c3ccc(N4CCN(C)CC4)cc3)n2)cc1] | 0.21 |
| 41 | 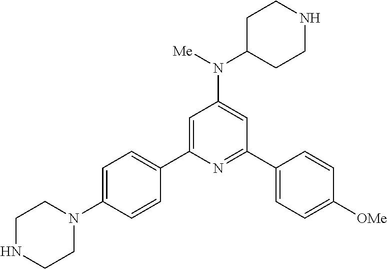 [SMILES: COc1ccc(-c2cc(N(C)C3CCNCC3)cc(-c3ccc(N4CCNCC4)cc3)n2)cc1] | 0.33 |
| 42 | 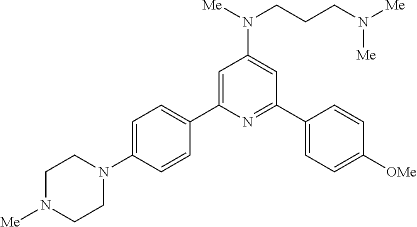 [SMILES: COc1ccc(-c2cc(N(C)CCCN(C)C)cc(-c3ccc(N4CCN(C)CC4)cc3)n2)cc1] | 0.46 |
| 43 | 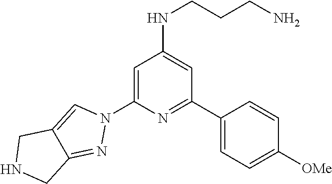 [SMILES: COc1ccc(-c2cc(NCCCN)cc(-n3cc4c(n3)CNC4)n2)cc1] | 0.46 |
| 44 | 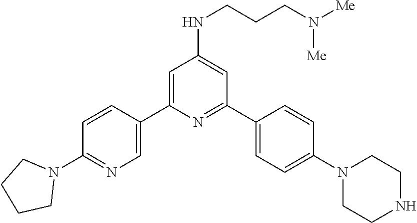 [SMILES: CN(C)CCCNc1cc(-c2ccc(N3CCNCC3)cc2)nc(-c2ccc(N3CCCC3)nc2)c1] | 7.95 |
| 45 | 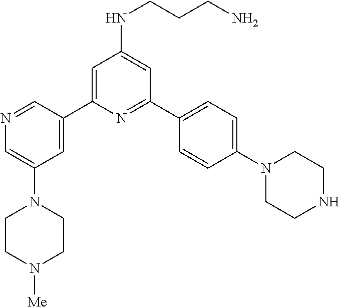 [SMILES: CN1CCN(c2cncc(-c3cc(NCCCN)cc(-c4ccc(N5CCNCC5)cc4)n3)c2)CC1] | 1.07 |
| 46 | 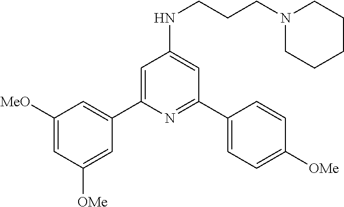 [SMILES: COc1ccc(-c2cc(NCCCN3CCCCC3)cc(-c3cc(OC)cc(OC)c3)n2)cc1] | 6.93 |
| 47 | 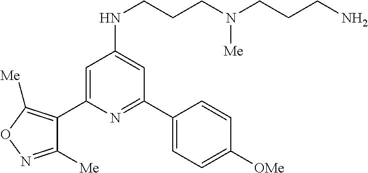 [SMILES: COc1ccc(-c2cc(NCCCN(C)CCCN)cc(-c3c(C)noc3C)n2)cc1] | 4.64 |
| 48 | 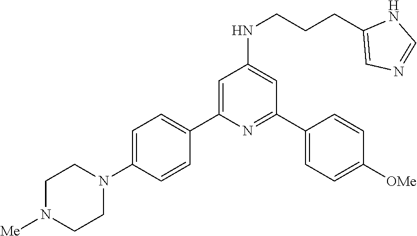 [SMILES: COc1ccc(-c2cc(NCCCc3cnc[nH]3)cc(-c3ccc(N4CCN(C)CC4)cc3)n2)cc1] | 3.77 |
| 49 | 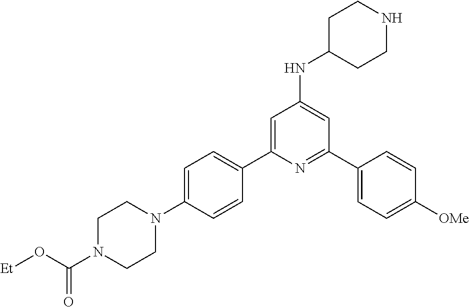 [SMILES: CCOC(=O)N1CCN(c2ccc(-c3cc(NC4CCNCC4)cc(-c4ccc(OC)cc4)n3)cc2)CC1] | 5.78 |
| 50 | 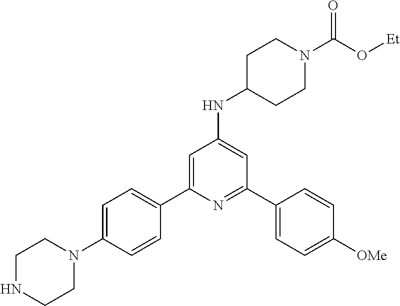 [SMILES: CCOC(=O)N1CCC(Nc2cc(-c3ccc(OC)cc3)nc(-c3ccc(N4CCNCC4)cc3)c2)CC1] | 9.49 |
| 51 | 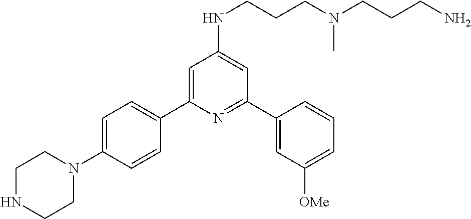 [SMILES: COc1cccc(-c2cc(NCCCN(C)CCCN)cc(-c3ccc(N4CCNCC4)cc3)n2)c1] | 0.64 |
| 52 | 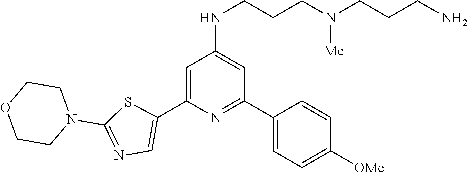 [SMILES: COc1ccc(-c2cc(NCCCN(C)CCCN)cc(-c3cnc(N4CCOCC4)s3)n2)cc1] | 5.58 |
| 53 | 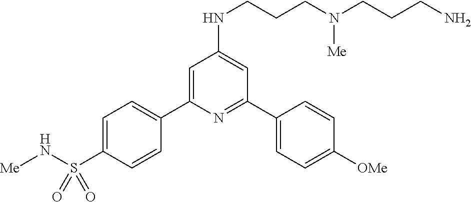 [SMILES: CNS(=O)(=O)c1ccc(-c2cc(NCCCN(C)CCCN)cc(-c3ccc(OC)cc3)n2)cc1] | 4.17 |
| 54 | 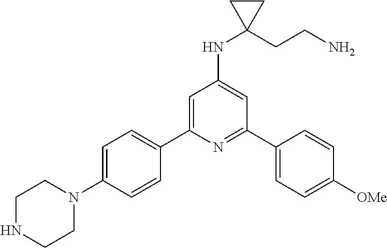 [SMILES: COc1ccc(-c2cc(NC3(CCN)CC3)cc(-c3ccc(N4CCNCC4)cc3)n2)cc1] | 3.56 |
| 55 | 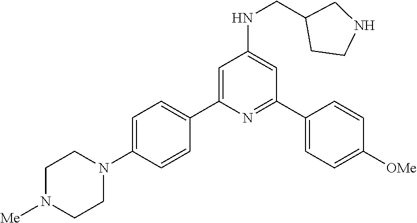 [SMILES: COc1ccc(-c2cc(NCC3CCNC3)cc(-c3ccc(N4CCN(C)CC4)cc3)n2)cc1] | 3.56 |
| 56 | 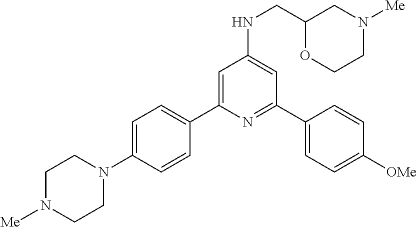 [SMILES: COc1ccc(-c2cc(NCC3CN(C)CCO3)cc(-c3ccc(N4CCN(C)CC4)cc3)n2)cc1] | 3.24 |
| 57 | 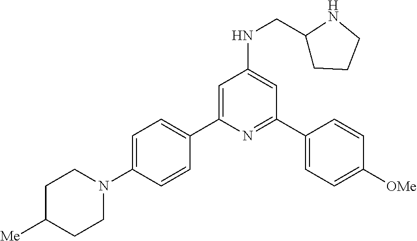 [SMILES: COc1ccc(-c2cc(NCC3CCCN3)cc(-c3ccc(N4CCC(C)CC4)cc3)n2)cc1] | 2.85 |
| 58 | 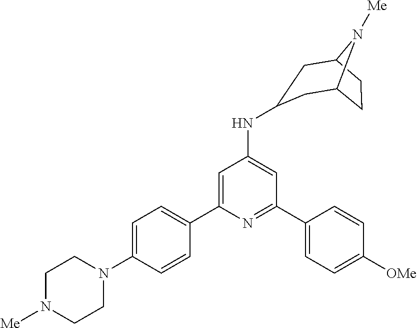 [SMILES: COc1ccc(-c2cc(NC3CC4CCC(C3)N4C)cc(-c3ccc(N4CCN(C)CC4)cc3)n2)cc1] | 2.85 |
| 59 | 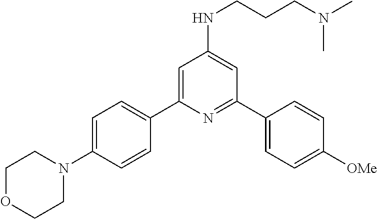 [SMILES: COc1ccc(-c2cc(NCCCN(C)C)cc(-c3ccc(N4CCOCC4)cc3)n2)cc1] | 2.04 |
| 60 | 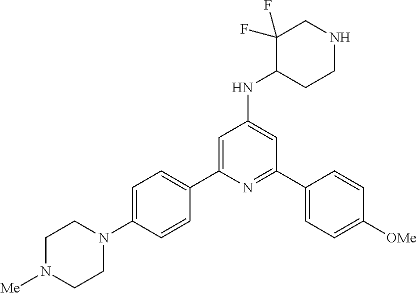 [SMILES: COc1ccc(-c2cc(NC3CCNCC3(F)F)cc(-c3ccc(N4CCN(C)CC4)cc3)n2)cc1] | 10.97 |
| 61 | 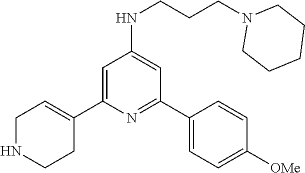 [SMILES: COc1ccc(-c2cc(NCCCN3CCCCC3)cc(C3=CCNCC3)n2)cc1] | 1.87 |
| 62 | 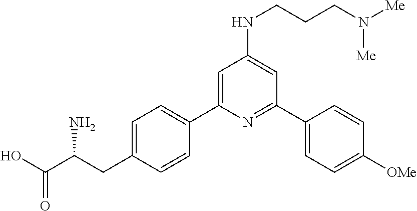 [SMILES: COc1ccc(-c2cc(NCCCN(C)C)cc(-c3ccc(C[C@@H](N)C(=O)O)cc3)n2)cc1] | 1.85 |
| 63 | 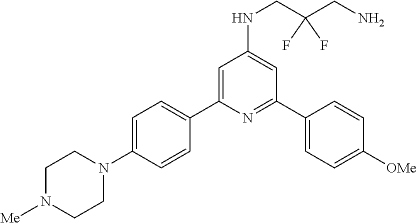 [SMILES: COc1ccc(-c2cc(NCC(F)(F)CN)cc(-c3ccc(N4CCN(C)CC4)cc3)n2)cc1] | 1.49 |
| 64 | 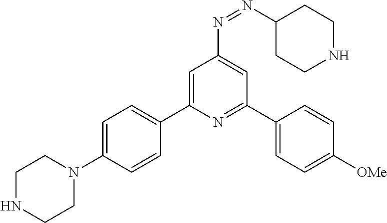 [SMILES: COc1ccc(-c2cc(/N=N\C3CCNCC3)cc(-c3ccc(N4CCNCC4)cc3)n2)cc1] | 1.48 |
| 65 | 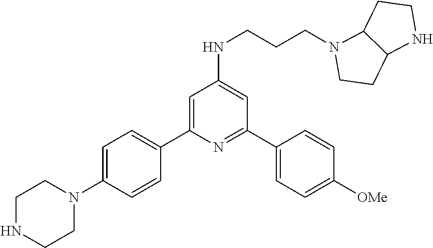 [SMILES: COc1ccc(-c2cc(NCCCN3CCC4NCCC43)cc(-c3ccc(N4CCNCC4)cc3)n2)cc1] | 1.32 |
| 66 | 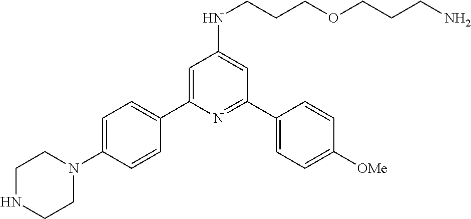 [SMILES: COc1ccc(-c2cc(NCCCOCCCN)cc(-c3ccc(N4CCNCC4)cc3)n2)cc1] | 1.20 |
| 67 | 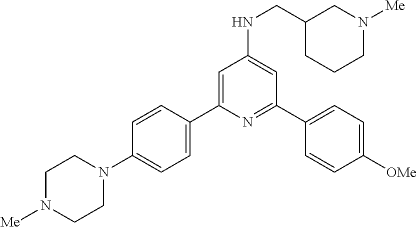 [SMILES: COc1ccc(-c2cc(NCC3CCCN(C)C3)cc(-c3ccc(N4CCN(C)CC4)cc3)n2)cc1] | 1.17 |
| 68 | 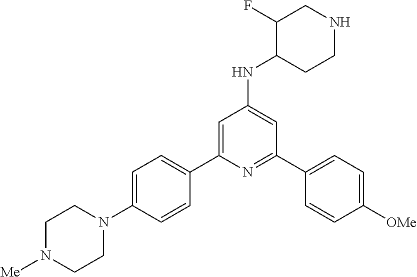 [SMILES: COc1ccc(-c2cc(NC3CCNCC3F)cc(-c3ccc(N4CCN(C)CC4)cc3)n2)cc1] | 0.89 |
| 69 | 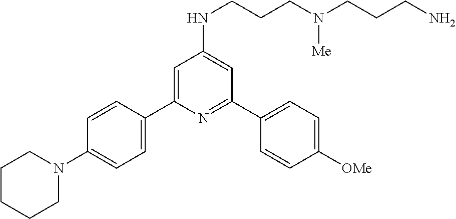 [SMILES: COc1ccc(-c2cc(NCCCN(C)CCCN)cc(-c3ccc(N4CCCCC4)cc3)n2)cc1] | 0.74 |
| 70 | 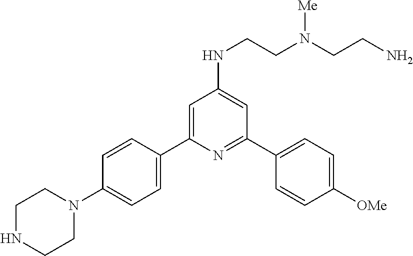 [SMILES: COc1ccc(-c2cc(NCCN(C)CCN)cc(-c3ccc(N4CCNCC4)cc3)n2)cc1] | 0.63 |
| 71 | 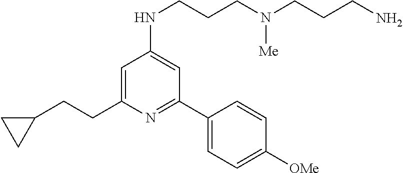 [SMILES: COc1ccc(-c2cc(NCCCN(C)CCCN)cc(CCC3CC3)n2)cc1] | 0.57 |
| 72 | 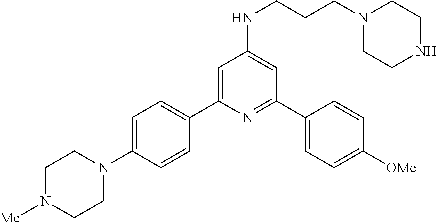 [SMILES: COc1ccc(-c2cc(NCCCN3CCNCC3)cc(-c3ccc(N4CCN(C)CC4)cc3)n2)cc1] | 0.55 |
| 73 | 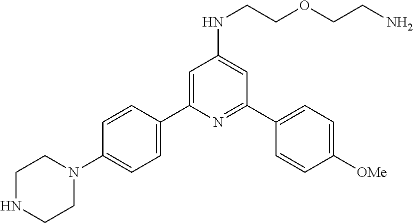 [SMILES: COc1ccc(-c2cc(NCCOCCN)cc(-c3ccc(N4CCNCC4)cc3)n2)cc1] | 0.49 |
| 74 | 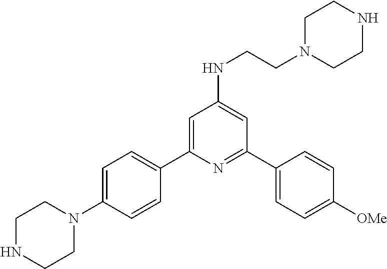 [SMILES: COc1ccc(-c2cc(NCCN3CCNCC3)cc(-c3ccc(N4CCNCC4)cc3)n2)cc1] | 0.49 |
| 75 | 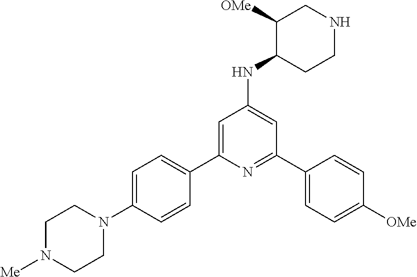 [SMILES: COc1ccc(-c2cc(N[C@@H]3CCNC[C@@H]3OC)cc(-c3ccc(N4CCN(C)CC4)cc3)n2)cc1] | 0.36 |
| 76 | 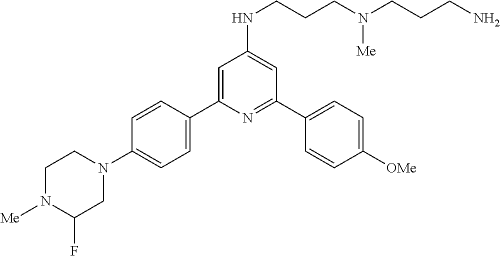 [SMILES: COc1ccc(-c2cc(NCCCN(C)CCCN)cc(-c3ccc(N4CCN(C)C(F)C4)cc3)n2)cc1] | 0.23 |
| 77 | 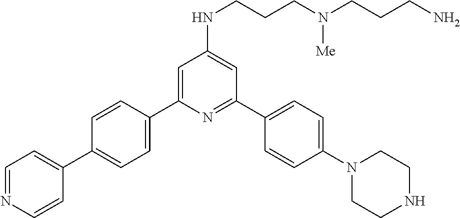 [SMILES: CN(CCCN)CCCNc1cc(-c2ccc(-c3ccncc3)cc2)nc(-c2ccc(N3CCNCC3)cc2)c1] | 0.13 |
| 78 | 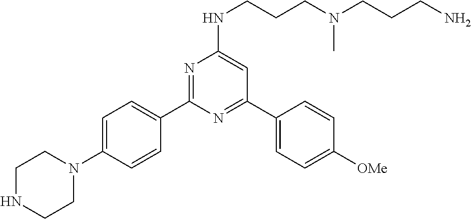 [SMILES: COc1ccc(-c2cc(NCCCN(C)CCCN)nc(-c3ccc(N4CCNCC4)cc3)n2)cc1] | 0.20 |
| 79 | 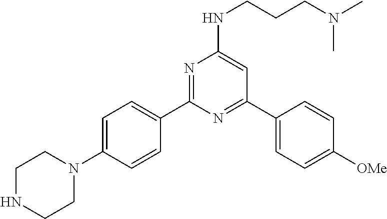 [SMILES: COc1ccc(-c2cc(NCCCN(C)C)nc(-c3ccc(N4CCNCC4)cc3)n2)cc1] | 1.09 |
| 80 | 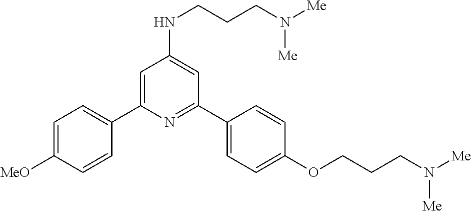 [SMILES: COc1ccc(-c2cc(NCCCN(C)C)cc(-c3ccc(OCCCN(C)C)cc3)n2)cc1] | 0.21 |
| 81 | 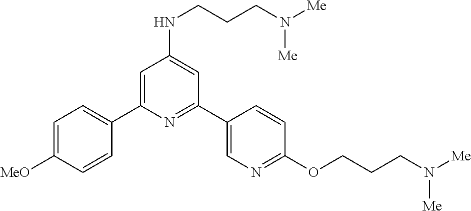 [SMILES: COc1ccc(-c2cc(NCCCN(C)C)cc(-c3ccc(OCCCN(C)C)nc3)n2)cc1] | 0.72 |
| 82 | 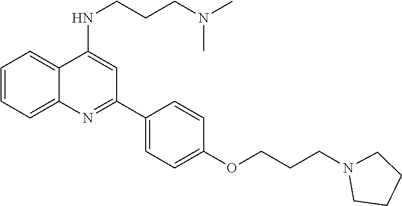 [SMILES: CN(C)CCCNc1cc(-c2ccc(OCCCN3CCCC3)cc2)nc2ccccc12] | 0.11 |
| 83 | 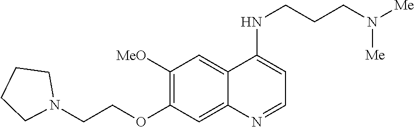 [SMILES: COc1cc2c(NCCCN(C)C)ccnc2cc1OCCN1CCCC1] | 0.19 |
| 84 | 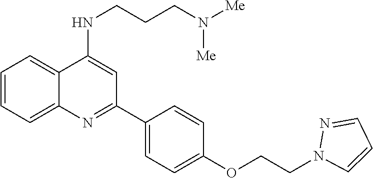 [SMILES: CN(C)CCCNc1cc(-c2ccc(OCCn3cccn3)cc2)nc2ccccc12] | 0.55 |
| 85 | 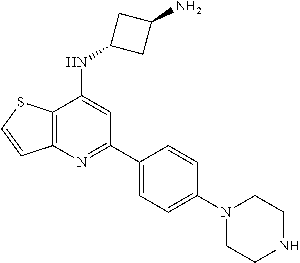 [SMILES: N[C@H]1C[C@H](Nc2cc(-c3ccc(N4CCNCC4)cc3)nc3ccsc23)C1] | 0.16 |
| 86 | 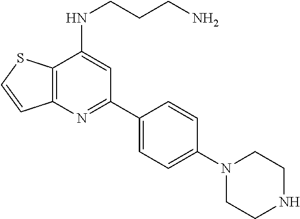 [SMILES: NCCCNc1cc(-c2ccc(N3CCNCC3)cc2)nc2ccsc12] | 0.20 |
| 87 | 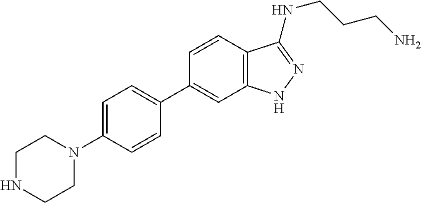 [SMILES: NCCCNc1n[nH]c2cc(-c3ccc(N4CCNCC4)cc3)ccc12] | 0.15 |
| 88 | 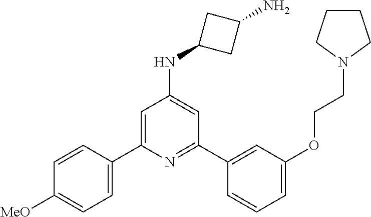 [SMILES: COc1ccc(-c2cc(N[C@H]3C[C@H](N)C3)cc(-c3cccc(OCCN4CCCC4)c3)n2)cc1] | 0.18 |
| 89 | 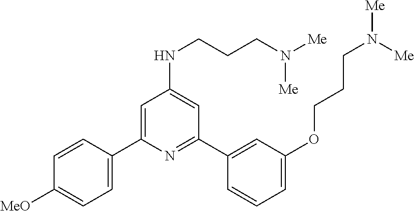 [SMILES: COc1ccc(-c2cc(NCCCN(C)C)cc(-c3cccc(OCCCN(C)C)c3)n2)cc1] | 0.17 |
| 90 | 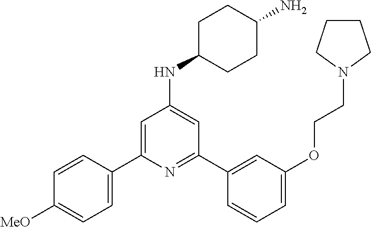 [SMILES: COc1ccc(-c2cc(N[C@H]3CC[C@H](N)CC3)cc(-c3cccc(OCCN4CCCC4)c3)n2)cc1] | 0.09 |
| 91 | 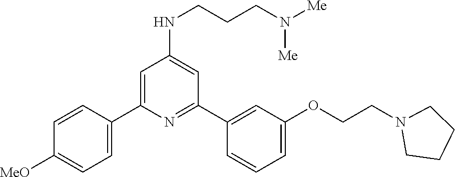 [SMILES: COc1ccc(-c2cc(NCCCN(C)C)cc(-c3cccc(OCCN4CCCC4)c3)n2)cc1] | 0.16 |
| 92 | 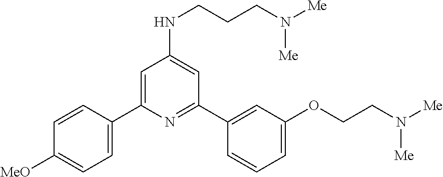 [SMILES: COc1ccc(-c2cc(NCCCN(C)C)cc(-c3cccc(OCCN(C)C)c3)n2)cc1] | 0.34 |
| 93 | 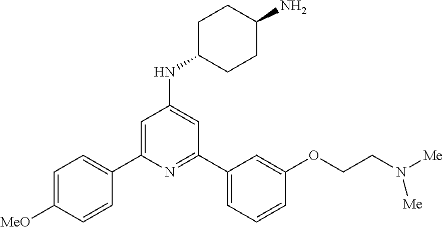 [SMILES: COc1ccc(-c2cc(N[C@H]3CC[C@H](N)CC3)cc(-c3cccc(OCCN(C)C)c3)n2)cc1] | 0.11 |
| 94 | 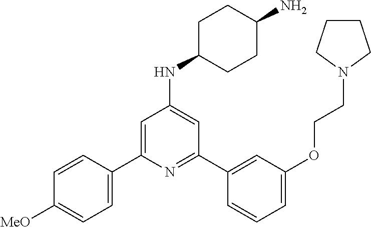 [SMILES: COc1ccc(-c2cc(N[C@H]3CC[C@@H](N)CC3)cc(-c3cccc(OCCN4CCCC4)c3)n2)cc1] | 0.49 |
| 95 | 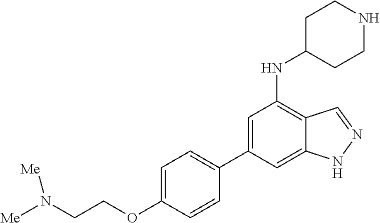 [SMILES: CN(C)CCOc1ccc(-c2cc(NC3CCNCC3)c3cn[nH]c3c2)cc1] | 2.40 |
| 96 | 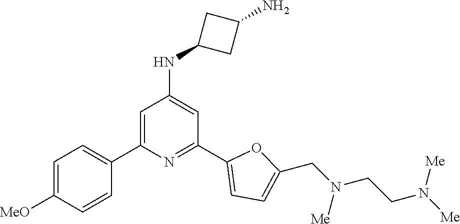 [SMILES: COc1ccc(-c2cc(N[C@H]3C[C@H](N)C3)cc(-c3ccc(CN(C)CCN(C)C)o3)n2)cc1] | 2.40 |

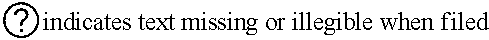

### EXAMPLES

The examples provided below provide representative methods for preparing exemplary compounds of the present invention. The skilled practitioner will know how to substitute the appropriate reagents, starting materials and purification methods known to those skilled in the art, in order to prepare the compounds of the present invention. ¹H-NMR spectra were obtained on a Varian Mercury 300-MHz NMR. Purity (%) and mass spectral data were determined with a Waters Alliance 2695 HPLC/MS (Waters Symmetry C₁₈, 4.6×75 mm, 3.5 μm) with a 2996 diode array detector from 210-400 nm.

General Method 1

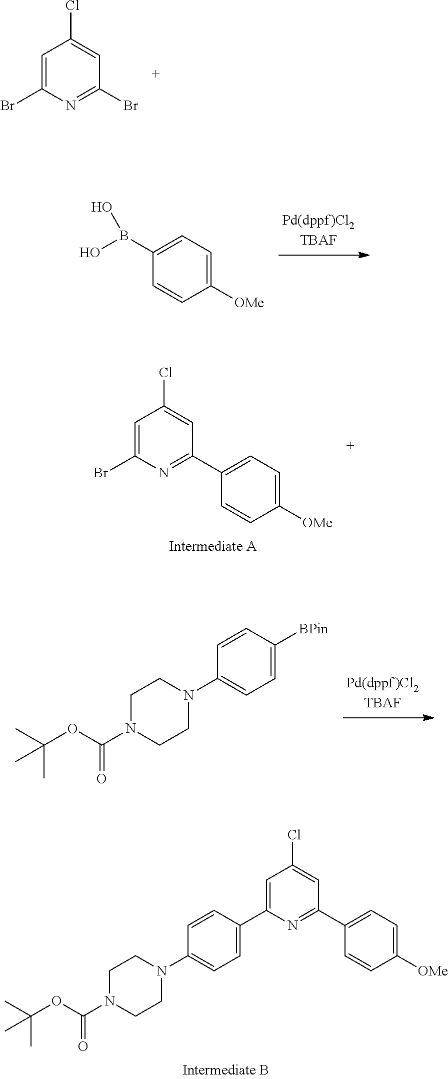

Intermediate A: 2,4-dichloro-6-(4-methoxyphenyl)pyridine. To a stirring solution of 2,6 dibromo-4-chloropyridine (3.09 g, 11.4 mmol), 4-methoxyphenylboronic acid (1.73 g, 11.4 mmol), and potassium carbonate (6.3 g, 45.6 mmol) were dissolved in dioxane/water (42/10.5 mL), the mixture was degassed by gentle flow of nitrogen via canula, followed by the addition of 1,1′-bis(diphenylphosphino)ferrocene] dichloropalladium(II) (93 mg, 0.11 mmol) and tetrabutyl ammonium bromide (368 mg, 1.1 mmol). The reaction mixture was heated to 40° C. for 90 minutes. The reaction was diluted with water and the aqueous layer extracted with ethyl acetate three times, the combined organic layers were dried over sodium sulfate and evacuated onto silica gel. Purified via column chromatography 125 g SiO₂ eluting with Hexanes to provide 0.77 g (23%) of 2-bromo-4-chloro-6-(4-methoxyphenyl)pyridine as a white solid. MS m z (M+H) 299.94.

Intermediate B: tert-butyl 4-(4-(4-chloro-6-(4-methoxyphenyl)pyridin-2-yl)phenyl)piperazine-1-carboxylate. To a solution of 2-bromo-4-chloro-6-(4-methoxyphenyl)pyridine (200 mg, 0.67 mmol) and tert-Butyl 4-[4-(4,4,5,5-tetramethyl-1,3,2-dioxaborolan-2-yl)phenyl]piperazine-1-carboxylate (272 mg, 0.70 mmol) were dissolved in 1,4-dioxane (5.3 mL), hereto was added potassium carbonate (2M, 1.35 mL, 0.70 mmol) the mixture was degassed by gentle flow of nitrogen via canula, followed by the addition of 1,1′-bis(diphenylphosphino)ferrocene]dichloropalladium(II) (5.5 mg, 0.007 mmol) and tetrabutyl ammonium bromide (22 mg, 0.07 mmol). The reaction mixture was heated for 14 hours at 90° C., cooled and partitioned between ethyl acetate and brine, combined organics were dried over sodium sulfate and concentrated in vacuo. The crude product was purified on silica gel eluting with a gradient of ethyl acetate in hexanes. Recovered 113 mg (35%) of tert-butyl 4-(4-(4-chloro-6-(4-methoxyphenyl)pyridin-2-yl)phenyl)piperazine-1-carboxylate as an off white solid. MS m z (M+H) 480.33.

General Method 2

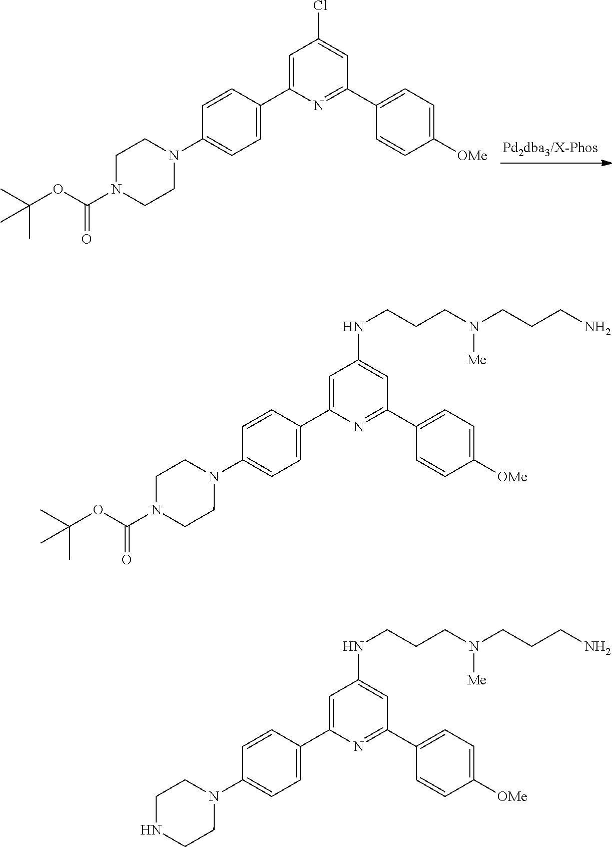 [SMILES: COc1ccc(-c2cc(Cl)cc(-c3ccc(N4CCN(C(=O)OC(C)(C)C)CC4)cc3)n2)cc1 ; COc1ccc(-c2cc(NCCCN(C)CCCN)cc(-c3ccc(N4CCN(C(=O)OC(C)(C)C)CC4)cc3)n2)cc1 ; COc1ccc(-c2cc(NCCCN(C)CCCN)cc(-c3ccc(N4CCNCC4)cc3)n2)cc1]

Example 2. N-{3-[(3-aminopropyl)(methyl)amino]propyl}-2-(4-methoxyphenyl)-6-[4-(piperazin-1-yl)phenyl]pyridin-4-amine. To a solution of tert-butyl 4-(4-(4-chloro-6-(4-methoxyphenyl)pyridin-2-yl)phenyl)piperazine-1-carboxylate (113 mg, 0.24 mmol) in t-butanol was added N¹-(3-aminopropyl)-N¹-methylpropane-1,3-diamine (38 mg, 0.26 mmol), the solutions was degassed via canula for 10 minutes, and hereto was added Pd₂dba₃ (11 mg, 0.0125 mmol), X-Phos (17 mg, 0.036 mmol) and sodium-tert-butoxide (46 mg, 0.48 mmol), the reaction vessel was briefly flushed with nitrogen, sealed and heated to 90° C. for 14 hours. The crude cooled reaction mixture was filtered through a plug of celite washing with ethyl acetate, concentrated in vacuo and purified on silica gel eluting with a gradient of methanol in dichloromethane. Recovered tert-butyl 4-{4-[4-({3-[(3-aminopropyl)(methyl)amino]propyl}amino)-6-(4-methoxyphenyl)pyridin-2-yl]phenyl}piperazine-1-carboxylate as a pale foam 30 mg (21%).

tert-butyl 4-{4-[4-({3-[(3-aminopropyl)(methyl)amino]propyl}amino)-6-(4-methoxyphenyl)pyridin-2-yl]phenyl}piperazine-1-carboxylate (30 mg, 0.05 mmol) was dissolved in anhydrous dichloromethane and hereto was added HCl in dioxane (2N, 0.100 mL). Stirred at ambient temperature for 1 hour. Concentrated in vacuo to afford N-{3-[(3-aminopropyl)(methyl)amino]propyl}-2-(4-methoxyphenyl)-6-[4-(piperazin-1-yl)phenyl]pyridin-4-amine as a white solid. MS m z 489.57 (M+H). ¹H NMR (300 MHz, METHANOL-d₄) δ=7.96-7.83 (m, 2H), 7.82-7.70 (m, 2H), 7.30-7.19 (m, 2H), 7.18-7.10 (m, 2H), 7.06-6.96 (m, 2H), 3.95-3.85 (m, 3H), 3.67-3.55 (m, 7H), 3.50-3.36 (m, 7H), 3.14-3.05 (m, 2H), 2.98-2.90 (m, 3H), 2.35-2.08 (m, 4H).

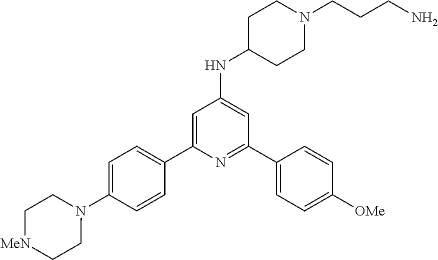 [SMILES: COc1ccc(-c2cc(NC3CCN(CCCN)CC3)cc(-c3ccc(N4CCN(C)CC4)cc3)n2)cc1]

Example 5. N-(1-(3-aminopropyl)piperidin-4-yl)-2-(4-methoxyphenyl)-6-(4-(4-methylpiperazin-1-yl)phenyl)pyridin-4-amine. tert-butyl 4-(2-(4-methoxyphenyl)-6-(4-(4-methylpiperazin-1-yl)phenyl)pyridin-4-ylamino)piperidine-1-carboxylate. 1-(4-(4-chloro-6-(4-methoxyphenyl)pyridin-2-yl)phenyl)-4-methylpiperazine (1.0 mg, 2.5 mmol), 1-tert-Butoxycarbonyl-4-aminopiperidine (0.626 mL, 3.13 mmol), sodium tert-butoxide (481 mg, 5.0), (1.4 mL, 1.4 mmol), tris(dibenzylideneacetone)dipalladium(0) (114 mg, 0.125 mmol), and DavePhos (118 mg, 0.3 mmol) were dissolved in dioxane (9.4 mL). The reaction mixture was heated to reflux and allowed to stir overnight. The reaction was cooled to ambient temperature, quenched by the addition of water, and the organic phase was extracted with dichloromethane three times. The combined organic layers were dried over sodium sulfate, filtered and evaporated in vacuo, and purified via column chromatography using 0-100% EtOAc/Hexanes to provide 1.21 mg (87%) of tert-butyl 4-((2-(4-methoxyphenyl)-6-(4-(4-methylpiperazin-1-yl)phenyl)pyridin-4-yl)amino)piperidine-1-carboxylate. MS m z 558.94.

2-(4-methoxyphenyl)-6-(4-(4-methylpiperazin-1-yl)phenyl)-N-(piperidin-4-yl)pyridin-4-amine. tert-Butyl 4-((2-(4-methoxyphenyl)-6-(4-(4-methylpiperazin-1-yl)phenyl)pyridin-4-yl)amino)piperidine-1-carboxylate (184 mg, 0.33 mmol) was treated with 4N HCl in dioxane (0.825 mL, 3.3 mmol) and stirred at ambient temperature for 1 h, at which point the reaction was judged to be complete by LC/MS. The reaction mixture was concentrated in vacuo to provide 2-(4-methoxyphenyl)-6-(4-(4-methylpiperazin-1-yl)phenyl)-N-(piperidin-4-yl)pyridin-4-amine in quantitative yield as a tetra-hydrochloric acid salt. MS m z 458.77. ¹H NMR (300 MHz, DMSO-d₆) δ ppm 7.82-7.96 (m, 2H), 7.67-7.82 (m, 1H), 7.10-7.21 (m, 4H), 6.96-7.10 (m, 2H), 3.97-4.07 (m, 2H), 3.81-3.88 (m, 3H), 3.60-3.80 (m, 2H), 3.43-3.55 (m, 2H), 3.08-3.29 (m, 3H), 2.94-3.08 (m, 1H), 2.76-2.85 (m, 2H), 2.14-2.28 (m, 1H), 2.00-2.12 (m, 1 H), 1.70-1.86 (m, 1H), 1.12-1.24 (m, 1H).

N-(1-(3-aminopropyl)piperidin-4-yl)-2-(4-methoxyphenyl)-6-(4-(4-methylpiperazin-1-yl)phenyl)pyridin-4-amine. 2-(4-methoxyphenyl)-6-(4-(4-methylpiperazin-1-yl)phenyl)-N-(piperidin-4-yl)pyridin-4-amine (137 mg, 0.3 mmol) and N-tert-butyl 3-aminopropanal carboxylate (104 mg, 0.6 mmol) were dissolved in 1,2-dichloroethane (3.0 mL) followed by the addition of 10 drops glacial acetic acid and sodium triacetoxyborohydride (127 mg, 0.6 mmol). The reaction was stirred at ambient temperature overnight before being quenched by the addition of water. The organic layer was extracted with dichloromethane three times, dried over sodium sulfate, filtered and evaporated in vacuo, and purified via column chromatography using 0-10% MeOH/DCM to provide 229 mg as a mixture of tert-butyl (3-((4-((2-(4-methoxyphenyl)-6-(4-(4-methylpiperazin-1-yl)phenyl)pyridin-4-yl)amino)cyclohexyl)amino)propyl)carbamate and the bis-alkylated product 1,1-bis(3-((tert-butoxycarbonyl)amino)propyl)-4-((2-(4-methoxyphenyl)-6-(4-(4-methylpiperazin-1-yl)phenyl)pyridin-4-yl)amino)piperidin-1-ium, which was taken on without further purifications. Treated with 4N HCl in dioxane (0.93 mL, 3.7 mmol) and stirred at ambient temperature for 1 h. The reaction mixture was concentrated in vacuo and the residue was purified via reversed-phase preparative HPLC. The lyophilate was dissolved in 10% isopropanol/dichloromethane and treated with ammonium hydroxide. The organic layer was separated and dried over sodium sulfate, filtered and evaporated in vacuo. The residue was taken up in minimal dichloromethane and treated with 2N HCl in diethyl ether and concentrated in vacuo. The residue was dried overnight in a vacuum oven to provide 42 mg of the title compound as a bis-hydrochloric acid salt. MS m z 515.60 (M+H). ¹H NMR (300 MHz, CHLOROFORM-d) δ ppm 7.89-8.11 (m, 4H), 6.93-7.06 (m, 4H), 6.67-6.80 (m, 2H), 4.05-4.17 (m, 1H), 3.83-3.94 (m, 3H), 3.24-3.37 (m, 4H), 2.89-3.02 (m, 2H), 2.72-2.83 (m, 2 H), 2.57-2.62 (m, 12H), 2.41-2.49 (m, 2H), 2.35-2.38 (m, 3H), 2.06-2.23 (m, 3H), 1.62-1.75 (m, 12H).

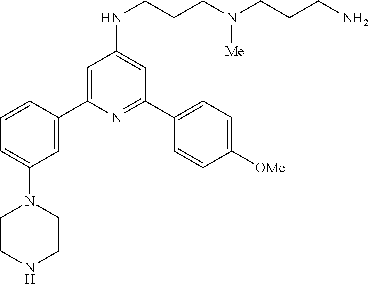 [SMILES: COc1ccc(-c2cc(NCCCN(C)CCCN)cc(-c3cccc(N4CCNCC4)c3)n2)cc1]

Example 7. N-{3-[(3-aminopropyl)(methyl)amino]propyl}-2-(4-methoxyphenyl)-6-[3-(piperazin-1-yl)phenyl]pyridin-4-amine. Using General Method 1 with 2,4-dichloro-6-(4-methoxyphenyl)pyridine (300 mg, 1.19 mmol) and 3-(4-(tert-butoxycarbonyl)piperazin-1-yl)phenylboronic acid (363 mg, 1.19 mmol), PddppfCl₂ (10 mg, 0.012 mmol), TBAB (39 mg, 0.12 mmol), potassium carbonate (2M, 2.4 mL, 4.0 mmol) afforded 1-(3-(4-chloro-6-(4-methoxyphenyl)pyridin-2-yl)phenyl)piperazine 110 mg as a white foam. MS m z 480.01 (M+H).

Using General Method 2 with 1-(3-(4-chloro-6-(4-methoxyphenyl)pyridin-2-yl)phenyl)piperazine (110 mg, 0.21 mmol) and N¹-(3-aminopropyl)-N¹-methylpropane-1,3-diamine (91 mg, 0.63 mmol), sodium tert-butoxide (40 mg, 0.42 mmol), Pd₂dba₃ (9.6 mg, 0.0105 mmol), X-Phos (15 mg, 0.032 mmol) afforded tert-butyl 4-{3-[4-({3-[(3-aminopropyl)(methyl)amino]propyl}amino)-6-(4-methoxyphenyl)pyridin-2-yl]phenyl}piperazine-1-carboxylate 47.2 mg as a greyish solid.

tert-butyl 4-{3-[4-({3-[(3-aminopropyl)(methyl)amino]propyl}amino)-6-(4-methoxyphenyl)pyridin-2-yl]phenyl}piperazine-1-carboxylate (47.2 mg, was dissolved in 1,4 dioxane and hereto was added HCl in dioxane (0.5 mL), stirred at room temperature for 14 hours, concentrated in vacuo, dissolved in water and lyophilized to a white solid (35.3 mg). MS m z 489.57 (M+H). ¹H NMR (300 MHz, DMSO-d₆) δ =8.04-7.91 (m, 1H), 7.86-7.72 (m, 1H), 7.55-7.32 (m, 2H), 7.29-7.01 (m, 5H), 3.95-3.79 (m, 3H), 3.65-3.47 (m, 7H), 3.28-3.07 (m, 8H), 3.00-2.83 (m, 2H), 2.79-2.69 (m, 3H), 2.20-1.93 (m, 4H).

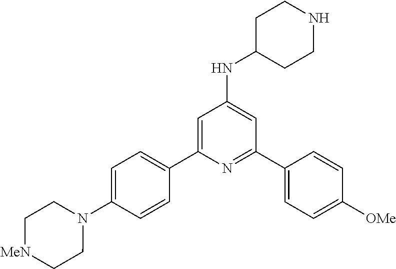 [SMILES: COc1ccc(-c2cc(NC3CCNCC3)cc(-c3ccc(N4CCN(C)CC4)cc3)n2)cc1]

Example 9. 2-(4-methoxyphenyl)-6-(4-(4-methylpiperazin-1-yl)phenyl)-N-(piperidin-4-yl)pyridin-4-amine. Using General Method 2 0.1-(4-(4-chloro-6-(4-methoxyphenyl)pyridin-2-yl)phenyl)-4-methylpiperazine (1.0 mg, 2.5 mmol), 1-tert-Butoxycarbonyl-4-aminopiperidine (0.626 mL, 3.13 mmol), sodium tert-butoxide (481 mg, 5.0), (1.4 mL, 1.4 mmol), Pd₂dba₃ (114 mg, 0.125 mmol), and DavePhos (118 mg, 0.3 mmol) were dissolved in dioxane (9.4 mL). 1.21 mg (87%) of tert-butyl 4-((2-(4-methoxyphenyl)-6-(4-(4-methylpiperazin-1-yl)phenyl)pyridin-4-yl)amino)piperidine-1-carboxylate. MS m z 558.94 (M+H).

tert-Butyl 4-((2-(4-methoxyphenyl)-6-(4-(4-methylpiperazin-1-yl)phenyl)pyridin-4-yl)amino)piperidine-1-carboxylate (184 mg, 0.33 mmol) was treated with 4N HCl in dioxane (0.825 mL, 3.3 mmol) and stirred at ambient temperature for 1 h, at which point the reaction was judged to be complete by LC/MS. The reaction mixture was concentrated in vacuo to provide 2-(4-methoxyphenyl)-6-(4-(4-methylpiperazin-1-yl)phenyl)-N-(piperidin-4-yl)pyridin-4-amine in quantitative yield as a tetra-hydrochloric acid salt. MS m z 458.77 (M+H). ¹H NMR (300 MHz, DMSO-d₆) δ ppm 7.82-7.96 (m, 2H), 7.67-7.82 (m, 1H), 7.10-7.21 (m, 4H), 6.96-7.10 (m, 2H), 3.97-4.07 (m, 2H), 3.81-3.88 (m, 3H), 3.60-3.80 (m, 2H), 3.43-3.55 (m, 2H), 3.08-3.29 (m, 3H), 2.94-3.08 (m, 1H), 2.76-2.85 (m, 2H), 2.14-2.28 (m, 1H), 2.00-2.12 (m, 1 H), 1.70-1.86 (m, 1H), 1.12-1.24 (m, 1H).

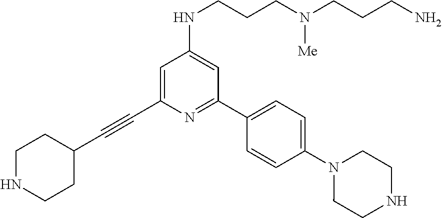 [SMILES: CN(CCCN)CCCNc1cc(C#CC2CCNCC2)nc(-c2ccc(N3CCNCC3)cc2)c1]

Example 11. N-{3-[(3-aminopropyl)(methyl)amino]propyl}-2-[4-(piperazin-1-yl)phenyl]-6-[2-(piperidin-4-yl)ethynyl]pyridin-4-amine. To a solution of 2,4,6 trichloropyridine (300 mg, 1.64 mmol) in TEA/THF(1.1.mL/2.2 mL) was added tert-butyl 4-ethynylpiperidine-1-carboxylate (0.343 mg, 1.64 mmol), the solution was degassed with a gentle flow of nitrogen for 15 minutes, followed by the addition of Pd(PPh₃)₂Cl₂ (58 mg, 0.082 mmol) and cupper(I)iodide (30 mg, 0.16 mmol). The reaction was stirred at 50° C. for 14 hours, cooled to room temperature and filtered through a plug of celite, concentrated in vacuo and purified on silica gel eluting with EtOAc in Hexanes, combined fractions afforded tert-butyl 4-[2-(4,6-dichloropyridin-2-yl)ethynyl]piperidine-1-carboxylate (223 mg).

Using General Method 1: tert-butyl 4-[2-(4,6-dichloropyridin-2-yl)ethynyl]piperidine-1-carboxylate (222 mg, 0.63 mmol) and tert-butyl 4(4-(4,4,5,5-tertramethyl-1,3,2-dioxaborolan-2-yl)phenyl)piperazine-1-carboxylate (243 mg, 0.63 mmol), PddppfCl₂ (5.1 mg, 0.006 mmol), TBAB (20.2 mg, 0.063 mmol) and potassium carbonate (1.25 mg, 2.5 mmol) afforded tert-butyl 4-{4-[6-(2-{1-[(tert-butoxy)(hydroxy)methyl]piperidin-4-yl}ethyl)-4-chloropiperidin-2-yl]phenyl}piperazine-1-carboxylate as a thick brown oil following purification on silica gel eluting with MeOH (with 5% ammonium hydroxide) in DCM. MS m z 581.48 (M+H).

Using General Method 2 tert-butyl 4-{4-[6-(2-{1-[(tert-butoxy)(hydroxy)methyl]piperidin-4-yl}ethyl)-4-chloropiperidin-2-yl]phenyl}piperazine-1-carboxylate (360 mg, 0.62 mmol), N¹-(3-aminopropyl)-N¹-methylpropane-1,3-diamine (270 mg, 1.86 mmol), Pd₂dba₃ (28 mg, 0.03 mmol), X-Phos (44 mg, 0.09 mmol) sodium tert-butoxide (119 mg, 1.24 mmol) afforded after purification on silica gel eluting with MeOH (with 5% ammonium hydroxide) in DCM (4-{2-[4-({3-[(3-aminopropyl)(methyl)amino]propyl}amino)-6-(4-{4-[(tert-butoxy)(hydroxy)methyl]piperazin-1-yl}cyclohexyl)piperidin-2-yl]ethyl}piperidin-1-yl)(tert-butoxy)methanol (78 mg). MS m z 690.76 (M+H).

(4-{2-[4-({3-[(3-aminopropyl)(methyl)amino]propyl}amino)-6-(4-{4-[(tert-butoxy)(hydroxy)methyl]piperazin-1-yl}cyclohexyl)piperidin-2-yl]ethyl}piperidin-1-yl)(tert-butoxy)methanol (78 mg 0.11 mmol) was dissolved in dioxane (1 mL), added HCl in dioxane (0.5 mL, 2 N), let stir at ambient overnight, concentrated in vacuo and partitioned between DCM and water, extracted three times with water, and freeze dried combined extracts. Isolated N-{3-[(3-aminopropyl)(methyl)amino]propyl}-2-[4-(piperazin-1-yl)phenyl]-6-[2-(piperidin-4-yl)ethynyl]pyridin-4-amine (22 mg). MS m z 490.62 (M+H). ¹H NMR (300 MHz, METHANOL-d₄) δ=7.82-7.61 (m, 1H), 7.20-7.04 (m, 1H), 6.90-6.70 (m, 1H), 6.45-6.24 (m, 1H), 3.75-3.52 (m, 4H), 3.45-3.34 (m, 1H), 3.34-3.18 (m, 3H), 3.11-2.93 (m, 1H), 2.70-2.54 (m, 2H), 2.35 (s, 3H), 2.04-1.67 (m, 2H).

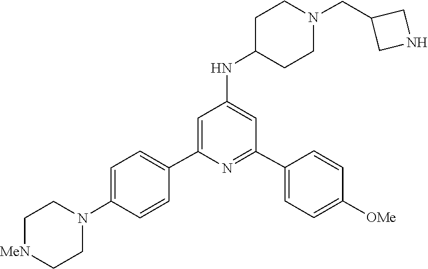 [SMILES: COc1ccc(-c2cc(NC3CCN(CC4CNC4)CC3)cc(-c3ccc(N4CCN(C)CC4)cc3)n2)cc1]

Example 12. N-(1-(azetidin-3-ylmethyl)piperidin-4-yl)-2-(4-methoxyphenyl)-6-(4-(4-methylpiperazin-1-yl)phenyl)pyridin-4-amine. tert-Butyl 2-((4-((2-(4-methoxyphenyl)-6-(4-(4-methylpiperazin-1-yl)phenyl)pyridin-4-yl)amino)piperidin-1-yl)methyl)azetidine-1-carboxylate (160 mg, 0.25 mmol) was dissolved in 50% trifluoroacetic acid/dichloromethane (1.0 mL) and stirred at ambient temperature for 1 h. The reaction mixture was concentrated in vacuo and the residue was dissolved in 10% iPrOH/DCM and treated with ammonium hydroxide. The organic layer was separated and dried over sodium sulfate, filtered and evaporated in vacuo, and concentrated in vacuo. The residue was taken up in minimal dichloromethane and treated with 2N HCl in diethyl ether and concentrated in vacuo. The residue was dried overnight in a vacuum oven to provide 141 mg of the title compound. MS m z 527.95 (M+H). ¹H NMR (300 MHz, DMSO-d₆) δ ppm 7.90 (br t, J=9.65 Hz, 4H), 6.86-7.18 (m, 6H), 6.85-6.97 (m, 1H), 5.70-5.75 (m, 1H), 3.93-4.10 (m, 2H), 3.83-3.91 (m, 2H), 3.74-3.83 (m, 3H), 2.71-2.80 (m, 3H), 1.98-2.22 (m, 2H), 1.67-1.99 (m, 1H).

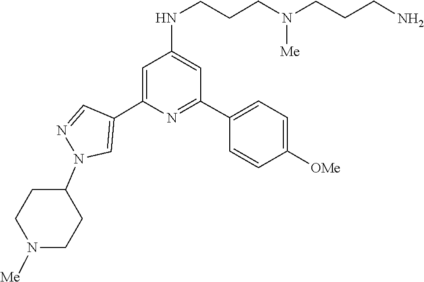 [SMILES: COc1ccc(-c2cc(NCCCN(C)CCCN)cc(-c3cnn(C4CCN(C)CC4)c3)n2)cc1]

Example 13. N-{3-[(3-aminopropyl)(methyl)amino]propyl}-2-(4-methoxyphenyl)-6-[1-(1-methylpiperidin-4-yl)-1H-pyrazol-4-yl]pyridin-4-amine. Using General Method 1: Intermediate A (320 mg, 1.07 mmol) and 1-methyl-4-(4-(4,4,5,5-tetramethyl-1,3,2-dioxaborolan-2-yl)-1H-pyrazol-1-yl)piperidine (328 mg, 1.13 mmol), PddppfCl₂ (9 mg, 0.01 mmol), TBAB (35 mg, 0.11 mmol) and potassium carbonate (2.14 mL, 4.28 mmol) in dioxane (8 mL), afforded after purification on silica gel eluting with a gradient of MeOH (with 5% ammonium hydroxide) in DCM 4-chloro-2-(4-methoxyphenyl)-6-(1-(1-methylpiperidin-4-yl)-1H-pyrazol-4-yl)pyridine as a yellow oil (240 mg).

Using General Method 2: 4-chloro-2-(4-methoxyphenyl)-6-(1-(1-methylpiperidin-4-yl)-1H-pyrazol-4-yl)pyridine (120 mg, 0.31 mmol), N¹-(3-aminopropyl)-N¹-methylpropane-1,3-diamine (137 mg, 0.97 mmol), Pd₂dba₃ (14 mg, 0.015 mmol), X-Phos (22 mg, 0.045 mmol) sodium tert-butoxide (60 mg, 0.64 mmol) Afforded after purification on silica gel eluting with MeOH (with 5% ammonium hydroxide) in DCM N-{3-[(3-aminopropyl)(methyl)amino]propyl}-2-(4-methoxyphenyl)-6-[1-(1-methylpiperidin-4-yl)-1H-pyrazol-4-yl]pyridin-4-amine (23 mg). MS m z 419.74 (M+H). ¹H NMR (300 MHz, METHANOL-d₄) δ=8.26-8.06 (m, 1H), 7.99-7.93 (m, 1H), 7.81-7.71 (m, 2H), 7.00-6.90 (m, 2H), 6.69 (s, 2H), 3.81 (s, 4H), 3.09-2.94 (m, 3H), 2.65-2.51 (m, 3H), 2.34-2.31 (m, 6H), 2.30-2.16 (m, 4H), 2.15-2.07 (m, 5H), 1.92-1.82 (m, 3H), 1.40-1.19 (m, 3H).

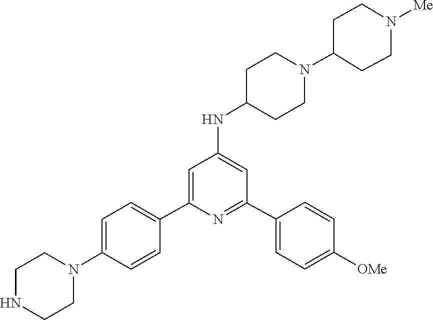 [SMILES: COc1ccc(-c2cc(NC3CCN(C4CCN(C)CC4)CC3)cc(-c3ccc(N4CCNCC4)cc3)n2)cc1]

Example 27. N-(2-(4-methoxyphenyl)-6-(4-(piperazin-1-yl)phenyl)pyridin-4-yl)-1′-methyl-[1,4′-bipiperidin]-4-amine. To a stirred solution of Intermediate B (100 mg, 0.27 mmol) in dioxane (4 mL), 1′-methyl-[1,4′-bipiperidin]-4-amine (266 mg, 1.35 mmol), tris(dibenzylideneacetone)dipalladium(0) (46 mg, 0.05 mmol), 2-dicyclohexylphosphino-2′-(N,N-dimethylamino)biphenyl (20 mg, 0.05 mmol) and sodium tert-butoxide (81 mg, 0.84 mmol) were added. The resulting mixture was purged with nitrogen for 10 minutes then heated to 90° C. for 15 hours. The reaction was cooled down to room temperature, diluted with methylene chloride (5 mL) and water (5 mL), filtered through celite, extracted with methylene chloride three times (5 mL). The organic layer was combined, dried over sodium sulfate, and concentrated by reduced pressure. The resulting solid was redissolved in methylene chloride (6 mL), trifluoroacetic acid (0.48 mL, 6.3 mmol) was added. The reaction was stirred at room temperature for 1 hour and concentrated by reduced pressure. The crude product was purified through HPLC (0-50% acetonitrile in water) to give N-(2-(4-methoxyphenyl)-6-(4-(piperazin-1-yl)phenyl)pyridin-4-yl)-1′-methyl-[1,4′-bipiperidin]-4-amine (99 mg, 68%). ¹H NMR (300 MHz, Chloroform-d) δ 8.12-7.92 (m, 4H), 7.14-6.89 (m, 4H), 6.87-6.66 (m, 2H), 4.08 (d, J=7.8 Hz, 1H), 3.85 (s, 3H), 3.55-3.40 (m, 1H), 3.29-3.16 (m, 4H), 3.12-3.00 (m, 4H), 2.97-2.85 (m, 4H), 2.53-2.36 (m, 2H), 2.27 (s, 3H), 2.20-2.04 (m, 2H), 1.99-1.78 (m, 6H), 1.67-1.54 (m, 4H). MS m/z (M+H) 542.00.

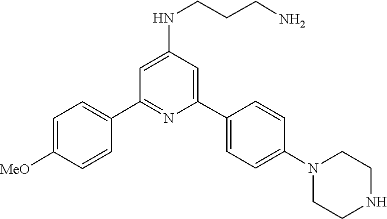 [SMILES: COc1ccc(-c2cc(NCCCN)cc(-c3ccc(N4CCNCC4)cc3)n2)cc1]

Example 31: N¹-(2-(4-methoxyphenyl)-6-(4-(piperazin-1-yl)phenyl)pyridin-4-yl)propane-1,3-diamine. To a stirred solution of Intermediate B (200 mg, 0.41 mmol) in dioxane (4 mL), tert-butyl (3-aminopropyl)carbamate (145 mg, 0.83 mmol), palladium(II) acetate (5 mg, 0.02 mmol), 2,2′-bis(diphenylphosphino)-1,1′-binaphthalene (25 mg, 0.04 mmol), tert-butyl carbonate (118 mg, 1.23 mmol) were added. The resulting mixture was purged with nitrogen for 10 minutes then heated to 70° C. for 15 hours. The reaction was cooled down to room temperature, diluted with methylene chloride and water, filtered through celite, extracted with methylene chloride three times. The organic layer was combined, dried over sodium sulfate, and concentrated by reduced pressure. The resulting solid was redissolved in methylene chloride (6 mL), trifluoroacetic acid (0.3 mL, 4.1 mmol) was added. The reaction was stirred at room temperature for 1 hour and concentrated by reduced pressure. The crude product was purified through HPLC (0-50% acetonitrile in water) to give N¹-(2-(4-methoxyphenyl)-6-(4-(piperazin-1-yl)phenyl)pyridin-4-yl)propane-1,3-diamine (110 mg, 64%). ¹H NMR (300 MHz, Chloroform-d) δ 8.08-7.85 (m, 4H), 7.05-6.86 (m, 4H), 6.86-6.56 (m, 2H), 4.82 (s, 1H), 3.86 (s, 3H), 3.37 (t, J=6.6 Hz, 2H), 3.22 (t, J=3.7 Hz, 4H), 3.06 (dd, J=6.4, 3.6 Hz, 4H), 2.91 (t, J=6.5 Hz, 2H), 1.82 (p, J=6.6 Hz, 2H). MS m/z (M+H) 418.52.

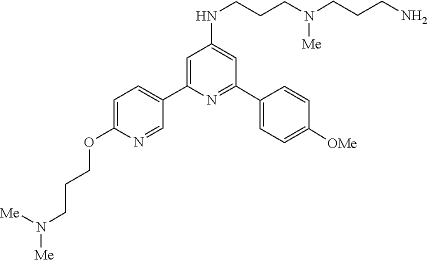 [SMILES: COc1ccc(-c2cc(NCCCN(C)CCCN)cc(-c3ccc(OCCCN(C)C)nc3)n2)cc1]

Example 35: N-{3-[(3-aminopropyl)(methyl)amino]propyl}-6′-[3-(dimethylamino)propoxy]-6-(4-methoxyphenyl)-[2,3′-bipyridin]-4-amine. Using General Method 1: Intermediate A (300 mg, 1.18 mmol) and N,N-dimethyl-3-((5-(4,4,5,5-tetramethyl-1,3,2-dioxaborolan-2-yl)pyridine-2-yl)oxy)propane-1-amine (362 mg, 1.18 mmol), PddppfCl₂ (8.6 mg, 0.012 mmol), TBAB (38 mg, 0.12 mmol) and potassium carbonate (653 mg, 4.7 mmol) in dioxane/water (8/1 mL), afforded after purification on silica gel eluting with a gradient of MeOH (with 5% ammonium hydroxide) in DCM 3-(5-(4-chloro-6-(4-methoxyphenyl)pyridin-2-yl)pyridin-2-yloxy)-N,N-dimethylpropan-1-amine (209 mg).

Using General Method 2: 3-(5-(4-chloro-6-(4-methoxyphenyl)pyridin-2-yl)pyridin-2-yloxy)-N,N-dimethylpropan-1-amine (100 mg, 0.25 mmol), N¹-(3-aminopropyl)-N¹-methylporpane-1,3-diamine (110 mg, 0.75 mmol), Pd₂dba₃ (11.5 mg, 0.012 mmol), X-Phos (18 mg, 0.038 mmol) sodium tert-butoxide (48 mg, 0.50 mmol) in t-BuOH afforded N-{3-[(3-aminopropyl)(methyl)amino]propyl}-6′-[3-(dimethylamino)propoxy]-6-(4-methoxyphenyl)-[2,3′-bipyridin]-4-amine (41 mg). MS m/z (M+H) 507.21. ¹H NMR (300 MHz, CHLOROFORM-d) δ=8.78 (d, J=2.3 Hz, 1H), 8.37-8.29 (m, 1H), 8.06-7.97 (m, 2H), 7.02-6.93 (m, 2H), 6.84-6.78 (m, 1H), 6.77-6.69 (m, 2H), 4.39 (s, 1H), 4.44-4.31 (m, 1H), 3.86 (s, 3H), 3.42-3.29 (m, 2H), 2.84-2.70 (m, 2H), 2.45 (d, J=7.6 Hz, 6H), 2.30-2.23 (m, 1OH), 2.08-1.92 (m, 2H), 1.89-1.77 (m, 3H), 1.73-1.54 (m, 4H).

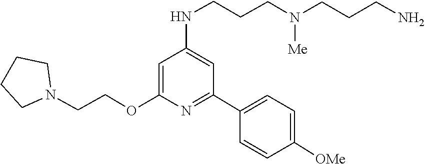 [SMILES: COc1ccc(-c2cc(NCCCN(C)CCCN)cc(OCCN3CCCC3)n2)cc1]

Example 37: N-{3-[(3-aminopropyl)(methyl)amino]propyl}-2-(4-methoxyphenyl)-6-[2-(pyrrolidin-1-yl)ethoxy]pyridin-4-amine. Intermediate A (200 mg, 0.67 mmol) and 1-(2-hydroxyethyl)pyrrolidine (79 mL, 0.67 mmol) were dissolved in anhydrous THF(3 mL) and cooled on a wet ice bath, NaHMDS (0.37 mL, 0.7 mmol) was added slowly. The reaction mixture was warmed to room temperature and heated 14 hours at 50° C. Quenched with saturated ammonium chloride and extracted three time with EtOAc, combined organics were washed with brine, dried over sodium sulfate and concentrated in vacuo. Purified on silica gel eluting with methanol with 5% ammonium hydroxide in DCM. Recovered 2-(2-(pyrrolidin-1-yl)ethoxy)-4-chloro-6-(4-methoxyphenyl)pyridine (125 mg).

Using General Method 2 2-(2-(pyrrolidin-1-yl)ethoxy)-4-chloro-6-(4-methoxyphenyl)pyridine (109 mg, 0.33 mmol), N¹-(3-aminopropyl)-N¹-methylporpane-1,3-diamine (143 mg, 0.98 mmol), Pd₂dba₃ (15 mg, 0.017 mmol), X-Phos (23.6 mg, 0.0495 mmol) sodium tert-butoxide (63 mg, 0.66 mmol) in t-BuOH. Reverse phase HPLC purification ACN/Water/TFA afforded N-{3-[(3-aminopropyl)(methyl)amino]propyl}-2-(4-methoxyphenyl)-6-[2-(pyrrolidin-1-yl)ethoxy]pyridin-4-amine; tris(trifluoroacetic acid) (15 mg).

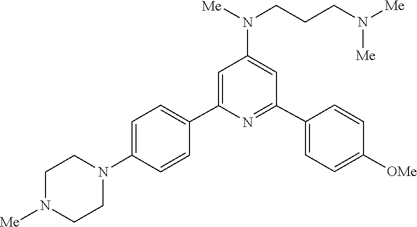 [SMILES: COc1ccc(-c2cc(N(C)CCCN(C)C)cc(-c3ccc(N4CCN(C)CC4)cc3)n2)cc1]

Example 42: N-(3-(dimethylamino)propyl)-2-(4-methoxyphenyl)-N-methyl-6-(4-(4-methylpiperazin-1-yl)phenyl)pyridin-4-amine. Using General Method 2 1-(4-(4-chloro-6-(4-methoxyphenyl)pyridin-2-yl)phenyl)-4-methylpiperazine (100 mg, 0.25 mmol) N¹-(3-aminopropyl)-N¹-methylporpane-1,3-diamine (112 mg, 0.76 mmol), Pd₂dba₃ (11 mg, 0.013 mmol), X-Phos (18 mg, 0.036 mmol) sodium tert-butoxide (48 mg, 0.50 mmol) in t-BuOH, afforded N-(3-(dimethylamino)propyl)-2-(4-methoxyphenyl)-N-methyl-6-(4-(4-methylpiperazin-1-yl)phenyl)pyridin-4-amine (12 mg). MS m/z (M+H) 475.01.

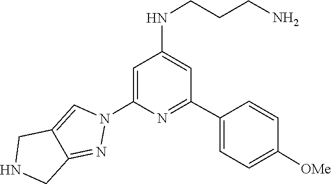 [SMILES: COc1ccc(-c2cc(NCCCN)cc(-n3cc4c(n3)CNC4)n2)cc1]

Example 43. Common Intermediate A (213 mg, 0.8 mmol), tert-Butyl 2H,4H,5H,6H-pyrrolo[3,4-c]pyrazole-5-carboxylate (213 mg, 1.0 mmol), and cesium carbonate (546 mg, 1.7 mmol) were dissolved in acetonitrile (2.5 mL), and heated to 75° C. for 14 hours. The reaction mixture was concentrated in vacuo and partitioned between water and DCM, extracted three time, the combined organic layers were dried over sodium sulfate, filtered and evaporated in vacuo, purified via silica gel chromatography using 0-25% EtOAc/Hexanes to provide 166 mg (49%) of tert-butyl 2-(4-chloro-6-(4-methoxyphenyl)pyridin-2-yl)-4,6-dihydropyrrolo[3,4-c]pyrazole-5(2H)-carboxylate. MS m z 427.26 (M+H).

tert-Butyl 2-(4-chloro-6-(4-methoxyphenyl)pyridin-2-yl)-4,6-dihydropyrrolo[3,4-c]pyrazole-5(2H)-carboxylate (166 mg, 0.39 mmol), tert-butyl (3-aminopropyl)carbamate (203 mg, 1.17 mmol), sodium tert-butoxide (75 mg, 0.78 mmol), DavePhos (18 mg, 0.047 mmol), and tris(dibenzylideneacetone)dipalladium(0) (18 mg, 0.02 mmol) were dissolved in dioxane (1.5 mL). The reaction mixture was heated to reflux for 14 hours. The reaction was cooled to ambient temperature, and partitioned between water and DCM. The combined organic layers were dried over sodium sulfate, filtered and evaporated in vacuo, and purified via column chromatography using 0-100% EtOAc/Hexanes to provide 51 mg (23%) of tert-butyl 2-(4-((3-((tert-butoxycarbonyl)amino)propyl)amino)-6-(4-methoxyphenyl)pyridin-2-yl)-4,6-dihydropyrrolo[3,4-c]pyrazole-5(2H)-carboxylate. MS m z 565.45 (M+H).

tert-Butyl 2-(4-((3-((tert-butoxycarbonyl)amino)propyl)amino)-6-(4-methoxyphenyl)pyridin-2-yl)-4,6-dihydropyrrolo[3,4-c]pyrazole-5(2H)-carboxylate (51 mg, 0.09 mmol) was dissolved in DCM (1 mL) treated with 4N HCl in dioxane (0.45 mL, 1.8 mmol) and stirred at ambient temperature for 1 h. The reaction mixture was concentrated in vacuo and the residue was purified using reversed-phased preparative HPLC to provide the title compound (35 mg). MS m z 365.37 (M-Boc+H). ¹H NMR (300 MHz, DMSO-d₆) δ ppm 8.57-8.66 (m, 1H), 7.98-8.11 (m, 2H), 7.73-7.88 (m, 3H), 7.69-7.72 (m, 1H), 7.44-7.51 (m, 1H), 7.07-7.12 (m, 1H), 6.98-7.07 (m, 2H), 6.78-6.90 (m, 1H), 6.62-6.72 (m, 1H), 4.81-4.92 (m, 1H), 4.38-4.51 (m, 3H), 4.28-4.38 (m, 1H), 3.76-3.89 (m, 3H), 3.38-3.52 (m, 2H), 2.81-2.99 (m, 2H), 1.79-1.97 (m, 2H).

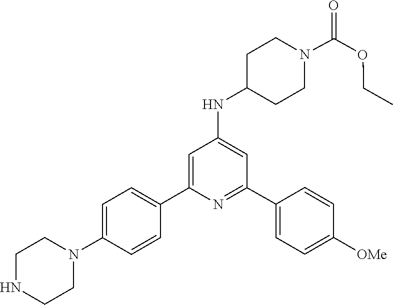 [SMILES: CCOC(=O)N1CCC(Nc2cc(-c3ccc(OC)cc3)nc(-c3ccc(N4CCNCC4)cc3)c2)CC1]

Example 49. Ethyl 4-(2-(4-methoxyphenyl)-6-(4-(piperazin-1-yl)phenyl)pyridin-4-ylamino)piperidine-1-carboxylate. Benzyl 4-(4-(4-((1-(tert-butoxycarbonyl)piperidin-4-yl)amino)-6-(4-methoxyphenyl)pyridin-2-yl)phenyl)piperidine-1-carboxylate (270 mg, 0.4 mmol) was treated with 4N HCl (1 mL, 4.0 mmol) and stirred at ambient temperature for an hour. The reaction mixture was concentrated in vacuo to give benzyl 4-(4-(6-(4-methoxyphenyl)-4-(piperidin-4-ylamino)pyridin-2-yl)phenyl)piperazine-1-carboxylate hydrochloride (230 mg). MS m z 578.71 (M+H).

Benzyl 4-(4-(6-(4-methoxyphenyl)-4-(piperidin-4-ylamino)pyridin-2-yl)phenyl)piperidine-1-carboxylate hydrochloride salt (230 mg, 0.4 mmol) was dissolved in THE (2 mL) and treated with ethyl chloroformate (0.046 mL, 0.48 mmol) followed by the addition of Hünig's base (0.274 mL, 1.6 mmol). The reaction mixture was stirred overnight at ambient temperature before being added to water, and extracted with ethyl acetate two times the combined organics were washed with 1N HCl (aq), saturated sodium bicarbonate (aq), and brine. The combined organic phases were dried over sodium sulfate, filtered and concentrated in vacuo to provide benzyl 4-(4-(4-((1-(ethoxycarbonyl)piperidin-4-yl)amino)-6-(4-methoxyphenyl)pyridin-2-yl)phenyl)piperazine-1-carboxylate which was used without further purification.

Crude benzyl 4-(4-(4-((1-(ethoxycarbonyl)piperidin-4-yl)amino)-6-(4-methoxyphenyl)pyridin-2-yl)phenyl)piperazine-1-carboxylate (0.4 mmol) was dissolved in methanol (4 mL) and 10% Palladium on carbon (25 mg) was added. The reaction mixture was vacuum purged and backfilled with hydrogen three times and stirred vigorously overnight at ambient temperature and 1 atm hydrogen. The suspension was filtered through celite and washed with methanol and concentrated in vacuo. The crude residue was purified via reversed-phase HPLC to provide ethyl 4-(2-(4-methoxyphenyl)-6-(4-(piperazin-1-yl)phenyl)pyridin-4-ylamino)piperidine-1-carboxylate (140 mg). MS m z 516.62 (M+H). ¹H NMR (300 MHz, DMSO-d₆) δ ppm 7.81-7.95 (m, 2H), 7.65-7.81 (m, 2H), 7.09-7.21 (m, 4H), 7.00-7.08 (m, 1H), 6.81-6.95 (m, 1H), 5.73-5.76 (m, 1H), 3.90-4.09 (m, 5H), 3.80-3.88 (m, 3H), 3.46-3.56 (m, 3H), 3.18-3.30 (m, 3H), 2.91-3.07 (m, 2H), 1.85-2.00 (m, 2H), 1.28-1.45 (m, 2 H), 1.13-1.22 (m, 3H).

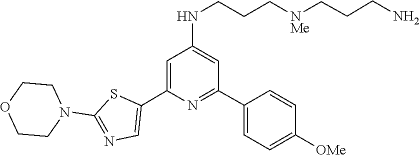 [SMILES: COc1ccc(-c2cc(NCCCN(C)CCCN)cc(-c3cnc(N4CCOCC4)s3)n2)cc1]

Example 52. N¹-(3-aminopropyl)-N³-(2-(4-methoxyphenyl)-6-(2-morpholinothiazol-5-yl)pyridin-4-yl)-N¹-methylpropane-1,3-diamine. 2,4-dichloro-6-(4-methoxyphenyl)pyridine (191 mg, 0.75 mmol 4-[5-(4,4,5,5-Tetramethyl-1,3,2-dioxaborolan-2-yl)-2-thiazolyl]morpholine (267 mg, 0.9 mmol), and 2M potassium carbonate (aq) (1.5 mL, 3.0 mmol) were dissolved in dioxane (3.0 mL) followed by the addition of Pd(dppf)Cl₂ CH₂Cl₂ (6 mg, 0.008 mmol) and tetrabutyl ammonium bromide (24 mg, 0.075 mmol). The reaction mixture was evacuated under vacuum and backfilled with nitrogen and heated to 90° C. for 14 hours. The reaction was cooled to ambient temperature and partitioned between water and DCM, aqueous was extracted three times with DCM, combined organic layers were dried over sodium sulfate, filtered, and purified via column chromatography using EtOAc/Hexanes to provide 110 mg of 4-(5-(4-chloro-6-(4-methoxyphenyl)pyridin-2-yl)thiazol-2-yl)morpholine. MS m/z 388.27 (M+H).

Using General Method 2: 4-(5-(4-chloro-6-(4-methoxyphenyl)pyridin-2-yl)thiazol-2-yl)morpholine (110 mg, 0.28 mmol), N¹-(3-aminopropyl)-N¹-methyl-1,3-propanediamine (0.137 mL, 0.85 mmol), X-Phos (20 mg, 0.04 mmol), tris(dibenzylideneacetone)dipalladium(0) (13 mg, 0.01 mmol), sodium tert-butoxide (55 mg, 0.57 mmol) in tert-butanol (1.9 mL). Purified via column chromatography using 0-10% MeOH/DCM with ammonium hydroxide to provide 84 mg of N¹-(3-aminopropyl)-N³-(2-(4-methoxyphenyl)-6-(2-morpholinothiazol-5-yl)pyridin-4-yl)-N¹-methylpropane-1,3-diamine. MS m z 497.48 (M+H). ¹H NMR (300 MHz, DMSO-d₆) δ ppm 7.86-7.95 (m, 2H), 7.73-7.78 (m, 1H), 6.96-7.04 (m, 2H), 6.72-6.82 (m, 2H), 6.51-6.58 (m, 1H), 3.77-3.82 (m, 3H), 3.67-3.75 (m, 4H), 3.53-3.63 (m, 2H), 3.39-3.48 (m, 4H), 3.13-3.23 (m, 3H), 2.27-2.43 (m, 4H), 2.11-2.17 (m, 3H), 1.71-1.78 (m, 2H), 1.62-1.72 (m, 2H), 1.44-1.58 (m, 2H), 1.19-1.28 (m, 1H).

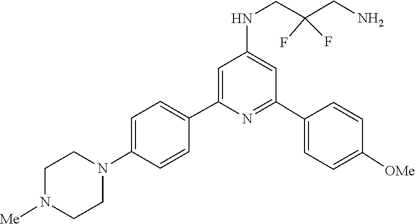 [SMILES: COc1ccc(-c2cc(NCC(F)(F)CN)cc(-c3ccc(N4CCN(C)CC4)cc3)n2)cc1]

Example 63. N-(3-amino-2,2-difluoropropyl)-2-(4-methoxyphenyl)-6-(4-(4-methylpiperazin-1-yl)phenyl)pyridin-4-amine. Using General Method 2 1-(4-(4-chloro-6-(4-methoxyphenyl)pyridin-2-yl)phenyl)-4-methylpiperazine (150 mg, 0.38 mmol) and 2,2-difluoropropane-1,3-diamine (209 mg, 1.14 mmol), X-Phos (27 mg, 0.06 mmol), tris(dibenzylideneacetone)dipalladium(0) (17 mg, 0.02 mmol) and sodium tert-butoxide (73 mg, 0.76 mmol). were dissolved in tert-butanol (1.9 mL). Filtered through celite concentrated in vacuo and purified on preparative HPLC CAN/water/TFA. Recovered N-(3-amino-2,2-difluoropropyl)-2-(4-methoxyphenyl)-6-(4-(4-methylpiperazin-1-yl)phenyl)pyridin-4-amine (17 mg). MS m z 497.48 (M+H).

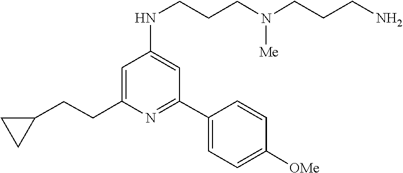 [SMILES: COc1ccc(-c2cc(NCCCN(C)CCCN)cc(CCC3CC3)n2)cc1]

Example 71. Common intermediate A (200 mg, 0.67 mmol) was dissolved in dioxane (2 mL) to this solution was added 2-((E)-2-cyclopropylvinyl)-4,4,5,5-tetramethyl-1,3,2-dioxaborolane (146 mL, 0.70 mmol), and potassium carbonate (0.703 mL, 1.41 mmol) the resulting mixture was degassed for 10 min with a flow of nitrogen via canula before adding Pd(PPh₃)₄(23 mg, 0.02 mmol). The reaction was heated to 90° C. for 14 hours, cooled to room temperature and filtered through a plug of celite washing with EtOAc, filtrate was concentrated in vacuo and purified on silica gel, eluting with a gradient of hexanes to 25% EtOAc in hexanes to afford 4-chloro-2-((E)-2-cyclopropylvinyl)-6-(4-methoxyphenyl)pyridine (160 mg). MS m z 497.48 (M+H).

Using General Method 2: 4-chloro-2-((E)-2-cyclopropylvinyl)-6-(4-methoxyphenyl)pyridine (140 mg, 0.49 mmol) N¹-(3-aminopropyl)-N¹-methyl-1,3-propanediamine (0.214 mL, 1.47 mmol), X-Phos (34 mg, 0.07 mmol), tris(dibenzylideneacetone)dipalladium(0) (22 mg, 0.025 mmol), sodium tert-butoxide (94 mg, 0.98 mmol) in tert-butanol (2.0 mL). Purified via column chromatography using 0-10% MeOH/DCM with ammonium hydroxide to provide N-{3-[(3-aminopropyl)(methyl)amino]propyl}-2-[(1E)-2-cyclopropylethenyl]-6-(4-methoxyphenyl)pyridin-4-amine (60 mg).

N-{3-[(3-aminopropyl)(methyl)amino]propyl}-2-[(1E)-2-cyclopropylethenyl]-6-(4-methoxyphenyl)pyridin-4-amine (56 mg, 0.14 mmol) was dissolved in MeOH and under nitrogen atmosphere Pd-C(11 mg) was added, the reaction vessel was stirred overnight under 1 atmosphere hydrogen. The reaction mixture was filtered through a plug of celite and purified on silica gel to afford N-{3-[(3-aminopropyl)(methyl)amino]propyl}-2-(2-cyclopropylethyl)-6-(4-methoxyphenyl)pyridin-4-amine (11 mg). ¹H NMR (300 MHz, CHLOROFORM-d) δ=7.90-7.81 (m, 2H), 6.99-6.88 (m, 2H), 6.65-6.57 (m, 1H), 6.27-6.20 (m, 1H), 5.44-5.31 (m, 1H), 3.82 (s, 3H), 3.28-3.16 (m, 2H), 2.87-2.74 (m, 4H), 2.64 (br s, 5H), 2.52-2.37 (m, 5H), 2.22 (s, 3H), 1.88-1.58 (m, 7H), 1.42-1.31 (m, 1H), 0.96-0.68 (m, 1H), 0.46-0.36 (m, 1H), 0.15-0.03 (m, 1H).

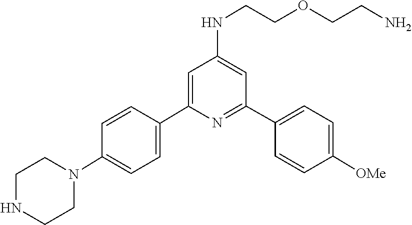 [SMILES: COc1ccc(-c2cc(NCCOCCN)cc(-c3ccc(N4CCNCC4)cc3)n2)cc1]

Example 73. N-(2-(2-aminoethoxy)ethyl)-2-(4-methoxyphenyl)-6-(4-(piperazin-1-yl)phenyl)pyridin-4-amine. Using General Method 2 tert-butyl 4-(4-(4-chloro-6-(4-methoxyphenyl)pyridin-2-yl)phenyl)piperazine-1-carboxylate (150 mg, 0.31 mmol) and 2-(2-aminoethoxy)ethanamine (98 mg, 0.94 mmol), X-Phos (22 mg, 0.05 mmol), tris(dibenzylideneacetone)dipalladium(0) (14 mg, 0.02 mmol) and sodium tert-butoxide (59 mg, 0.62 mmol) were dissolved in tert-butanol (1.5 mL), following purification on silica gel eluting with 0-10% MeOH/DCM with ammonium hydroxide, tert-butyl 4-(4-(4-(2-(2-aminoethoxy)ethylamino)-6-(4-methoxyphenyl)pyridin-2-yl)phenyl)piperazine-1-carboxylate (37.8 mg) was isolated.

tert-butyl 4-(4-(4-(2-(2-aminoethoxy)ethylamino)-6-(4-methoxyphenyl)pyridin-2-yl)phenyl)piperazine-1-carboxylate (35.6 mg, 0.065 mmol) was dissolved in DCM (2 mL) and hereto was added 2N HCl in dioxane (65 mL, 0.13 mmol), stirred at room temperature for 14 hours, concentrated in vacuo, dissolved in minimal amount of water and freeze dried to afford N-(2-(2-aminoethoxy)ethyl)-2-(4-methoxyphenyl)-6-(4-(piperazin-1-yl)phenyl)pyridin-4-amine (25 mg). MS m z 448.55 (M+H). ¹H NMR (300 MHz, METHANOL-d₄) δ=7.91-7.70 (m, 4H), 7.28-7.20 (m, 2H), 7.19-7.04 (m, 3H), 7.00-6.92 (m, 1H), 3.95-3.87 (m, 3H), 3.85-3.73 (m, 4H), 3.72-3.65 (m, 2H), 3.65-3.58 (m, 4H), 3.44-3.37 (m, 4H), 3.21-3.14 (m, 2H).

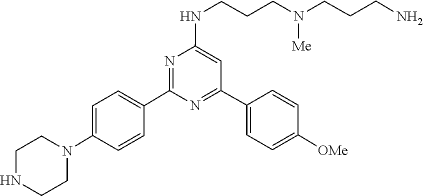 [SMILES: COc1ccc(-c2cc(NCCCN(C)CCCN)nc(-c3ccc(N4CCNCC4)cc3)n2)cc1]

Example 78. 2,4,6-trichloropyrimidine (500 mL, 4.35 mmol), tert-butyl 4-(4-(4,4,5,5-tetramethyl-1,3,2-dioxaborolan-2-yl)phenyl)piperazine-1-carboxylate (1.6 g, 4.13 mmol), and sodium carbonate (4.15 mL, 8.26 mmol) were dissolved in toluene and ethanol, and degassed for 15 min via canula, Pd(PPh₃)₄(238 mg, 0.21 mmol) was added and the reaction heated to 50° C. for 6 hours. Cooled to room temperature and concentrated in vacuo, partitioned between ethyl acetate and water, extracted three times, combined organics were washed with brine, dried over sodium sulfate, filtered and concentrated in vacuo. Purified on silica gel eluting with a gradient of ethyl acetate in hexanes, recovered tert-butyl 4-(4-(4,6-dichloropyrimidin-2-yl)phenyl)piperazine-1-carboxylate (856 mg).

tert-butyl 4-(4-(4,6-dichloropyrimidin-2-yl)phenyl)piperazine-1-carboxylate (450 mg, 1.11 mmol), 4-Methoxyphenyl boronic acid (168 mg, 1.11 mmol), sodium carbonate (1.10 mL, 2.22 mmol) were dissolved in toluene and ethanol, and degassed for 15 min via canula, Pd(PPh₃)₄(64 mg, 0.06 mmol) was added and the reaction heated to 90° C. for 3 hours, filtered through a plug of celite, concentrated and purified on silica gel eluting with ethyl acetate in hexanes, recovered tert-butyl 4-(4-(4-chloro-6-(4-methoxyphenyl)pyrimidin-2-yl)phenyl)piperazine-1-carboxylate (322 mg).

4-chloro-6-(4-methoxyphenyl)-2-(4-(piperazin-1-yl)phenyl)pyrimidine (322 mg, 0.67 mmol), N¹-(3-aminopropyl)-N¹-methyl-1,3-propanediamine (145 mg, 1.00 mmol) dissolved in IPA (2 mL) and heated to 160° C. for 10 min under microwave irradiation. Purified on silica gel eluting with DCM and methanol with ammonium hydroxide (5%). Recovered unreacted starting material and N-{3-[(3-aminopropyl)(methyl)amino]propyl}-2-(2-cyclopropylethyl)-tert-butyl 4-(4-(4-chloro-6-(4-methoxyphenyl)pyrimidin-2-yl)phenyl)piperazine-1-carboxylate (45 mg). MS m/z 419.43 (M+H).

N-{3-[(3-aminopropyl)(methyl)amino]propyl}-2-(2-cyclopropylethyl)-tert-butyl 4-(4-(4-chloro-6-(4-methoxyphenyl)pyrimidin-2-yl)phenyl)piperazine-1-carboxylate (45 mg) dissolved in DCM (1 mL) HCl in dioxane (0.5 mL, 2M) was added and the reaction stirred at room temperature for 12 hours. Concentrated in vacuo, dissolved in minimal amount of water, neutralized with sodium hydroxide and extracted into ethyl acetate, washed combined organics with brine, dried over sodium sulfate, concentrated in vacuo, dissolved in small amount of dioxane added water and freeze dried to afford N-{3-[(3-aminopropyl)(methyl)amino]propyl}-6-(4-methoxyphenyl)-2-[4-(piperazin-1-yl)phenyl]pyrimidin-4-amine (20 mg). MS m/z 490.55 (M+H). ¹H NMR (300 MHz, METHANOL-d₄) δ=8.05-8.05 (m, 1H), 8.16-7.98 (m, 3H), 7.41-7.37 (m, 1H), 7.08-6.95 (m, 4H), 3.86 (s, 3H), 3.61-3.51 (m, 2H), 3.28-3.22 (m, 4H), 3.01-2.94 (m, 3H), 2.70-2.59 (m, 2H), 2.58-2.50 (m, 2H), 2.49-2.40 (m, 2H), 2.26 (s, 3H), 1.97-1.83 (m, 2H), 1.73-1.60 (m, 2H).

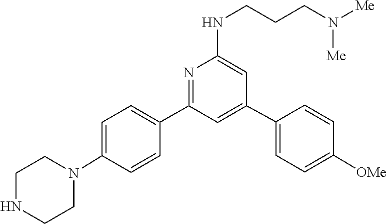 [SMILES: COc1ccc(-c2cc(NCCCN(C)C)nc(-c3ccc(N4CCNCC4)cc3)c2)cc1]

Example 79. N¹-(4-(4-methoxyphenyl)-6-(4-(piperazin-1-yl)phenyl)pyridin-2-yl)-N³,N³-dimethylpropane-1,3-diamine. To a stirred solution of 1-(4-(6-chloro-4-(4-methoxyphenyl)pyridin-2-yl)phenyl)piperazine (200 mg, 0.42 mmol) in dioxane (3 mL), N¹,N¹-dimethylpropane-1,3-diamine (43 mg, 0.84 mmol), palladium(II) acetate (5 mg, 0.02 mmol), 2,2′-bis(diphenylphosphino)-1,1′-binaphthalene (249 mg, 0.4 mmol), cesium carbonate (274 mg, 0.84 mmol) were added. The resulting mixture was purged with nitrogen for 10 minutes then heated to 70° C. for 15 hours. The reaction was cooled down to room temperature, diluted with methylene chloride and water, filtered through celite, extracted with methylene chloride three times. The organic layer was combined, dried over sodium sulfate, and concentrated by reduced pressure. The resulting solid was redissolved in methylene chloride (6 mL), trifluoroacetic acid (0.48 mL, 6.3 mmol) was added. The reaction was stirred at room temperature for 1 hour and concentrated by reduced pressure. The crude product was purified through HPLC (0-50% acetonitrile in water) to give N¹-(4-(4-methoxyphenyl)-6-(4-(piperazin-1-yl)phenyl)pyridin-2-yl)-N³,N³-dimethylpropane-1,3-diamine (67 mg, 36%). ¹H NMR (300 MHz, Chloroform-d) δ 8.03-7.84 (m, 2H), 7.76-7.52 (m, 2H), 7.13 (dd, J=4.5, 1.3 Hz, 1H), 7.08-6.89 (m, 5H), 6.50 (d, J=1.3 Hz, 1H), 3.83 (d, J=0.9 Hz, 3H), 3.49 (dt, J=11.6, 5.9 Hz, 4H), 3.26 (dq, J=6.1, 3.2 Hz, 4H), 3.08 (dd, J=6.4, 3.6 Hz, 4H), 2.64 (td, J=6.9, 3.2 Hz, 2H), 2.38 (s, 6H), 1.96 (h, J=6.8 Hz, 3H). MS m/z (M+H) 446.59.

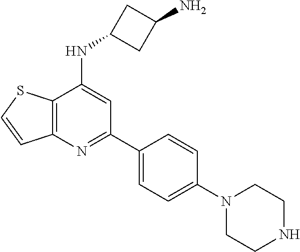 [SMILES: N[C@H]1C[C@H](Nc2cc(-c3ccc(N4CCNCC4)cc3)nc3ccsc23)C1]

Example 85. (1r,3r)-N1-(5-(4-(piperazin-1-yl)phenyl)thieno[3,2-b]pyridin-7-yl)cyclobutane-1,3-diamine. 5,7-Dichlorothieno[3,2-b]pyridine (102 mg, 0.5 mmol), (4-(4-(tert-butoxycarbonyl)piperazin-1-yl)phenyl)boronic acid pinacol ester (204 mg, 0.525 mmol), tetrakistriphenylphosphine palladium (17 mg, 0.015 mmol), aqueous 2M sodium carbonate (0.5 mL, 1 mmol) were dissolved in toluene (1 mL) and ethanol (1 mL). The reaction mixture was placed under vacuum and backfilled with nitrogen three times before being heated to 90° C. and stirred overnight. After cooling to ambient temperature, the reaction was diluted with water and dichloromethane and the layers were partitioned. The aqueous layer was washed (2x) with dichloromethane (10 mL), and the combined organic layers were dried over sodium sulfate, filtered, and concentrated in vacuo. The crude was purified via column chromatography using 0-100% EtOAc/hexanes to provide 191 mg (89%) of tert-butyl 4-(4-(7-chlorothieno[3,2-b]pyridin-5-yl)phenyl)piperazine-1-carboxylate. MS: m/z 430.42 (M+H).

Tert-butyl 4-(4-(7-chlorothieno[3,2-b]pyridin-5-yl)phenyl)piperazine-1-carboxylate (215 mg, 0.5 mmol), trans-N-Boc-1,3-diaminocyclobutane (279 mg, 1.5 mmol), palladium acetate (6 mg, 0.025 mmol), BINAP (34 mg, 0.055 mmol), and potassium phosphate tribasic (212 mg, 1.0 mmol) were dissolved in dioxane (1.9 mL) and water (0.2 mL). The reaction mixture was placed under vacuum and backfilled with nitrogen three times before being heated to reflux for 14 hours. After cooling to ambient temperature, the reaction was diluted with water and dichloromethane, the aqueous layer was washed (2×) with dichloromethane (10 mL), and the combined organic layers were dried over sodium sulfate, filtered, and concentrated in vacuo. The crude was purified via column chromatography using 0-100% EtOAc/hexanes to provide 120 mg (41%) of tert-butyl 4-(4-(7-(((1r,3r)-3-((tert-butoxycarbonyl)amino)cyclobutyl)amino)thieno[3,2-b]pyridin-5-yl)phenyl)piperazine-1-carboxylate. MS: m/z 580.34 (M+H).

Tert-butyl 4-(4-(7-(((1r,3r)-3-((tert-butoxycarbonyl)amino)cyclobutyl)amino)thieno[3,2-b]pyridin-5-yl)phenyl)piperazine-1-carboxylate (120 mg, 0.2 mmol) was dissolved in dichloromethane (0.5 mL) and treated with hydrogen chloride, 4N in dioxane (0.78 mL, 3.1 mmol). The reaction was stirred for 1 hour at ambient temperature and then concentrated in vacuo. The mixture was then treated with ammonium hydroxide (˜4 mL) and extracted twice with 10% isopropanol/dichloromethane (10 mL), dried over sodium sulfate, filtered, and concentrated in vacuo to provide the free base. A solution of 2 N HCl in diethyl ether was added and concentrated in vacuo to give 91 mg of the title compound as a bis HCl salt. MS: m/z=380.43 (M+H). ¹H NMR (300 MHz, DMSO-d₆) S=7.88 (d, J=8.8 Hz, 2H), 7.85 (d, J=5.3 Hz, 1H), 7.38-7.32 (m, 1H), 6.96 (d, J=8.8 Hz, 2H), 6.85 (d, J=5.3 Hz, 1H), 6.59 (s, 1H), 4.35-4.19 (m, 1H), 3.57-3.45 (m, 1H), 3.08 (dd, J=3.8, 6.2 Hz, 4H), 2.87-2.74 (m, 4H), 2.28 (ddd, J=5.0, 7.5, 12.2 Hz, 3H), 2.16-2.00 (m, 2H).

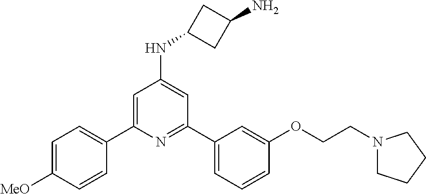 [SMILES: COc1ccc(-c2cc(N[C@H]3C[C@H](N)C3)cc(-c3cccc(OCCN4CCCC4)c3)n2)cc1]

Example 88: trans-N1-(2-(3-(2-(pyrrolidin-1-yl)ethoxy)phenyl)-6-(4-methoxyphenyl)pyridin-4-yl)cyclobutane-1,3-diamine: Using general method 1 2,4-Dichloro-6-(4-methoxyphenyl)pyridine (5.0 g, 19.685 mmol), 3-hydroxyphenylboronic acid (3.260 g, 23.62 mmol), PdCl₂dppf (1.44 g, 1.97 mmol) and K₃PO₄.7H₂O (13.3 g, 39.37 mmol) were suspended in 1,4-Dioxane (120 mL) and water (13 mL), to yield the title compound as brown solid (4.96 g, 81%). MS: m/z 3-(4-chloro-6-(4-methoxyphenyl)pyridin-2-yl)phenol (130 mg, 0.418 mmol), 1-(2-chloroethyl)pyrrolidine HCl salt (2.85 g, 16.75 mmol), Cs₂CO₃ (13.0 g, 40 mmol) and KI (100 mg) were suspended in anhydrous DMF (90 mL) and then stirred at 80° C. for 3 h and at rt overnight. Another 2.5 g of Cs₂CO₃ was added and stirred at 85° C. for 10 h. The mixture was cooled to rt and then diluted with EtOAc (100 ml) and washed with water 2×30 mL and brine 1 ×30 mL and then dried over anhydrous Na₂SO₄. The solvent was removed in vacuo. The crude product was loaded onto a 120 g silica ISCO column and purified by ISCO chromatography using DCM/MeOH (0-10%) to yield the title compound as brown oil (5.56 g, 85%). MS: m/z 409.62 (M+H). ¹H NMR (300 MHz, CHLOROFORM-d) δ ppm 8.06-8.10 (m, 2H) 7.72 (d, J=3.07 Hz, 1H) 7.59-7.66 (m, 3H) 7.39-7.42 (m, 1H) 6.99-7.04 (m, 3H) 4.22 (t, J=6.15 Hz, 2H) 3.88 (s, 3H) 2.94-2.99 (m, 2H) 2.67 (br. s., 4H) 1.80-1.87 (m, 4H) 2-(3-(2-(pyrrolidin-1-yl)ethoxy)phenyl)-4-chloro-6-(4-methoxyphenyl)pyridine (29 mg, 0.07 mmol), trans-3-Boc-amino cyclobutylamine (31 mg, 0.167 mmol), Pd₂dba₃ (6.5 mg, 0.007 mmol), XPhos (6.5 mg, 0.014 mmol) and NaOtBu (14 mg, 0.146 mmol) were placed in a 1-dram vial and then anhydrous tert-butanol (0.5 mL) was added. The mixture was purged with nitrogen by bubbling though a nitrogen stream for 1-2 min and then capped and stirred at 80° C. overnight.

The mixture was diluted with EtOAc (5 mL) and washed with water 2×1 mL. The solvent was removed in vacuo to yield the crude product. This material was used without further purification. The crude product was dissolved in a 1:1 mixture of TFA/Dichloromethane (0.5 mL) that contained a drop of water and then stirred at rt for 30 min. The solvent was removed in vacuo. The product was purified by Gilson using CH₃CN/H₂O/TFA mobile phase to yield the title compound as a brown oil (8.2 mg, 15%). MS: m/z 459.68 (M+H). ¹H NMR (300 MHz, DMSO-d₆) δ ppm 7.07-7.12 (m, 2H) 6.75-6.81 (m, 1H) 6.70 (d, J=7.82 Hz, 1H) 6.56 (t, J=7.92 Hz, 1H) 6.17-6.23 (m, 3H) 5.95 (q, J=2.15 Hz, 2H) 3.35-3.44 (m, 3H) 3.04 (s, 3H) 2.78-2.96 (m, 1H) 2.18 (t, J=5.57 Hz, 2H) 1.88-1.96 (m, 4H) 1.51 (t, J=6.45 Hz, 4H) 1.01-1.08 (m, 4 H).

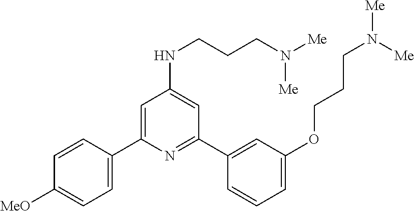 [SMILES: COc1ccc(-c2cc(NCCCN(C)C)cc(-c3cccc(OCCCN(C)C)c3)n2)cc1]

Example 89: 2-(3-(3-(dimethylamino)propoxy)phenyl)-N-(3-(dimethylamino)propyl)-6-(4-methoxyphenyl)pyridin-4-amine: 3-(4-chloro-6-(4-methoxyphenyl)pyridin-2-yl)phenol (130 mg, 0.418 mmol), 3-(dimethylamino)propanol (45 mg, 0.436 mmol) and PPh₃ (218 mg, 0.83 mmol) were dissolved in anhydrous THE (1 mL) and then cooled to 0° C. at an ice bath and then DIAD (168 mg, 0.83 mmol) was added dropwise. The mixture was stirred at 0° C. for 30 min and at rt overnight. The solvent was removed in vacuo. The product was loaded onto a 4 g silica ISCO column and purified by ISCO chromatography using Hexanes/EtOAc (0-100%) to yield the title compound as brown solid (56 mg, 33%). MS: m/z 397.65 (M+H). 3-(3-(4-chloro-6-(4-methoxyphenyl)pyridin-2-yl)phenoxy)-N,N-dimethylpropan-1-amine (28 mg, 0.07 mmol), 3-dimethylaminopropylamine (18 mg, 0.17 mmol), Pd₂dba₃ (6.5 mg, 0.007 mmol), XPhos (6.5 mg, 0.014 mmol) and NaOtBu (14 mg, 0.146 mmol) were placed in a 1-dram vial and then anhydrous tert-butanol (0.5 mL) was added. The mixture was purged with nitrogen by bubbling through a nitrogen stream for 1-2 min and then capped and stirred at 80° C. overnight.

The mixture was diluted with EtOAc (5 mL) and washed with water 2×1 mL. The solvent was removed in vacuo to yield the crude product, which was purified by Gilson using CH₃CN/H₂O/TFA mobile phase to yield the title compound as a brown oil (820 mg, 35%). MS: m/z 463.74 (M+H). ¹H NMR (300 MHz, METHANOL-d₄) δ ppm 7.76-7.84 (m, 4H) 7.16 (t, J=8.79 Hz, 4H) 6.95 (d, J=3.60 Hz, 2H) 4.21 (t, J=5.86 Hz, 2H) 3.89 (s, 3H) 3.54-3.59 (m, 2H) 3.33-3.41 (m, 2H) 3.26-3.32 (m, 2H) 2.96 (s, 6H) 2.92 (s, 6H) 2.27-2.30 (m, 2H) 2.12-2.16 (m, 2H).

### Example 93: trans-N1-(2-(3-(2-(dimethylamino)ethoxy)phenyl)-6-(4-methoxyphenyl)pyridin-4-yl)cyclohexane-1,4-diamine

2-(3-(4-chloro-6-(4-methoxyphenyl)pyridin-2-yl)phenoxy)-N,N-dimethylethanamine (27 mg, 0.07 mmol), trans-4-Boc-amino cyclohexylamine (36 mg, 0.17 mmol), Pd₂dba₃ (6.5 mg, 0.007 mmol), XPhos (6.5 mg, 0.014 mmol) and NaOtBu (14 mg, 0.146 mmol) were placed in a 1-dram vial and then anhydrous tert-butanol (0.5 mL) was added. The mixture was purged with nitrogen by bubbling though a nitrogen stream for 1-2 min and then capped and stirred at 80° C. overnight. The mixture was diluted with EtOAc (5 mL) and washed with water 2×1 mL. The solvent was removed in vacuo to yield the crude product. This material was used without further purification. The crude product was dissolved in a 1:1 mixture of TFA/Dichloromethane (0.5 mL) that contained a drop of water and then stirred at rt for 30 min. The solvent was removed in vacuo. The product was purified by Gilson using CH₃CN/H₂O/TFA mobile phase to yield the title compound as a brown oil (9.0 mg, 16%). MS: m/z 461.71 (M+H). ¹H NMR (300 MHz, METHANOL-d₄) δ ppm 7.72-7.84 (m, 4H) 7.22-7.26 (m, 2H) 7.14 (dd, J=8.79, 2.93 Hz, 2H) 6.98 (s, 1H) 6.88 (s, 1H) 4.47 (br. s., 2H) 3.90 (s, 3H) 3.76-3.79 (m, 1H), 3.65 (t, J=4.69 Hz, 2H) 3.16-3.19 (m, 1H) 3.01 (s, 6H) 2.16-2.23 (m., 4H) 1.57-1.72 (m, 4H)

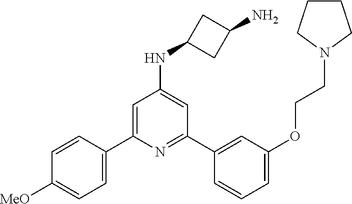 [SMILES: COc1ccc(-c2cc(N[C@H]3C[C@@H](N)C3)cc(-c3cccc(OCCN4CCCC4)c3)n2)cc1]

### Example 94: Cis-N1-(2-(3-(2-(pyrrolidin-1-yl)ethoxy)phenyl)-6-(4-methoxyphenyl)pyridin-4-yl)cyclobutane-1,3-diamine triple TFA salt

Step 1: 2-(3-(2-(pyrrolidin-1-yl)ethoxy)phenyl)-4-chloro-6-(4-methoxyphenyl)pyridine (G3) (100 mg, 0.244 mmol), cis-3-Boc-amino cyclobutylamine (93 mg, 0.5 mmol), Pd₂dba₃ (23 mg, 0.025 mmol), XPhos (24 mg, 0.05 mmol) and NaOtBu (48 mg, 0.5 mmol) were placed in a 1-dram vial and then anhydrous tert-butanol (1.6 mL) was added. The mixture was purged with nitrogen by bubbling though a nitrogen stream for 1-2 min and then capped and stirred at 80° C. overnight. The mixture was diluted with EtOAc (5 mL) and washed with water 2×1 mL, brine 1 ×1 ml, dried over MgSO₄, the solvent was removed in vacuo to yield the crude product, which was purified by a silica ISCO column (12 g) using Hex/EtOAc to yield the pure product (110 mg, 80%). MS: m/z 559.67 (M+H)

Step 2: The product from step 1 (110 mg) was dissolved in a 1:1 mixture of TFA/Dichloromethane (1 mL) that contained a drop of water and then stirred at room temperature for 30 min. The solvent was removed in vacuo. The product was purified by reverse phase chromatography using CH₃CN/H₂O/TFA mobile phase to yield the title compound as a fluffy white solid (5 mg). MS: m/z 459.68 (M+H). ¹H NMR (300 MHz, Methanol-d₄) δ ppm 7.90 (d, 2H) 7.58 (s, 1H) 7.52 (d, J=8.21 Hz, 1H) 7.37 (t, J=7.92 Hz, 1H) 6.99-7.03 (m, 3H) 6.80 (s, 2H) 4.23 (t, J=5.57 Hz, 2H), 3.84 (s, 3H) 3.70-3.79 (m, 1H) 3.27-3.38 (m, 1H) 3.02 (t, J=5.57 Hz, 2H) 2.82-2.93 (m, 2H) 2.70-2.80 (m, 4H) 1.74-1.94 (m, 6H).

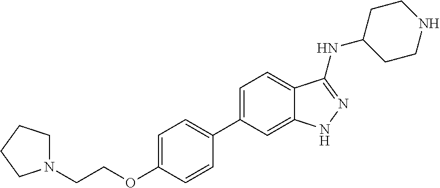 [SMILES: c1cc(-c2ccc3c(NC4CCNCC4)n[nH]c3c2)ccc1OCCN1CCCC1]

Example 95: 6-(4-(2-(pyrrolidin-1-yl)ethoxy)phenyl)-N-(piperidin-4-yl)-1H-indazol-3-amine. 6-bromo-1H-indazol-3-amine (1.0 g, 4.7 mmol), tert-butyl 4-oxopiperidine-1-carboxylate (0.94 g, 4.7 mmol), acetic acid (0.29 mL, 4.7 mmol), sodium triacetoxyborohydride (2.0 g, 9.4 mmol), and dichloroethane (20 mL) were combined and stirred at ambient temperature for 18 hours. The reaction was diluted with water (20 mL) and saturated aqueous sodium bicarbonate (20 mL). The organic layer was separated, concentrated in vacuo and purified on gel eluting with a gradient of ethyl acetate in hexanes to yield tert-butyl 4-(6-bromo-1H-indazol-3-ylamino)piperidine-1-carboxylate as a light purple solid, 0.66 g (35%). MS m z 395.35/397.26. tert-Butyl 4-(6-bromo-1H-indazol-3-ylamino)piperidine-1-carboxylate (100 mg, 0.25 mmol), 1-(2-(4-(4,4,5,5-tetramethyl-1,3,2-dioxaborolan-2-yl)phenoxy)ethyl)pyrrolidine (120 mg, 0.37 mmol), 1,1′-bis(diphenylphosphino)ferrocene]dichloropalladium(II) (8 mg, 0.01 mmol), potassium carbonate (105 mg, 0.76 mmol), water (0.3 mL), and dioxane (1 mL) were combined and heated in a sealed vial for 14 hours at 80° C. After cooling the reaction was partitioned between ethyl acetate and brine and the organic layer concentrated in vacuo. Reverse phase HPLC purification ACN/Water/TFA afforded tert-butyl 4-(6-(4-(2-(pyrrolidin-1-yl)ethoxy)phenyl)-1H-indazol-3-ylamino)piperidine-1-carboxylate: trifluoroacetic acid salt. MS m z 506.32. The resulting solid was dissolved in methylene chloride (5 mL) and trifluoroacetic acid (1 mL) was added. The reaction was stirred at room temperature for 3 hours and concentrated by reduced pressure. The crude product was purified by reverse phase HPLC purification ACN/Water/TFA to give the title compound 6-(4-(2-(pyrrolidin-1-yl)ethoxy)phenyl)-N-(piperidin-4-yl)-1H-indazol-3-amine: tris trifluoroacetic acid salt, 52 mg (28%). MS m z 406.24 (M+H). ¹H NMR (300 MHz, METHANOL-d₄) δ=7.86 (d, J=9.4 Hz, 1H), 7.74-7.61 (m, 2H), 7.49 (s, 1H), 7.38-7.31 (m, 1H), 7.18-7.10 (m, 2H), 4.46-4.32 (m, 2H), 3.91-3.64 (m, 5H), 3.59-3.37 (m, 2H), 3.28-3.06 (m, 4H), 2.41-2.28 (m, 2H), 2.27-2.00 (m, 4H), 1.98-1.80 (m, 2H).

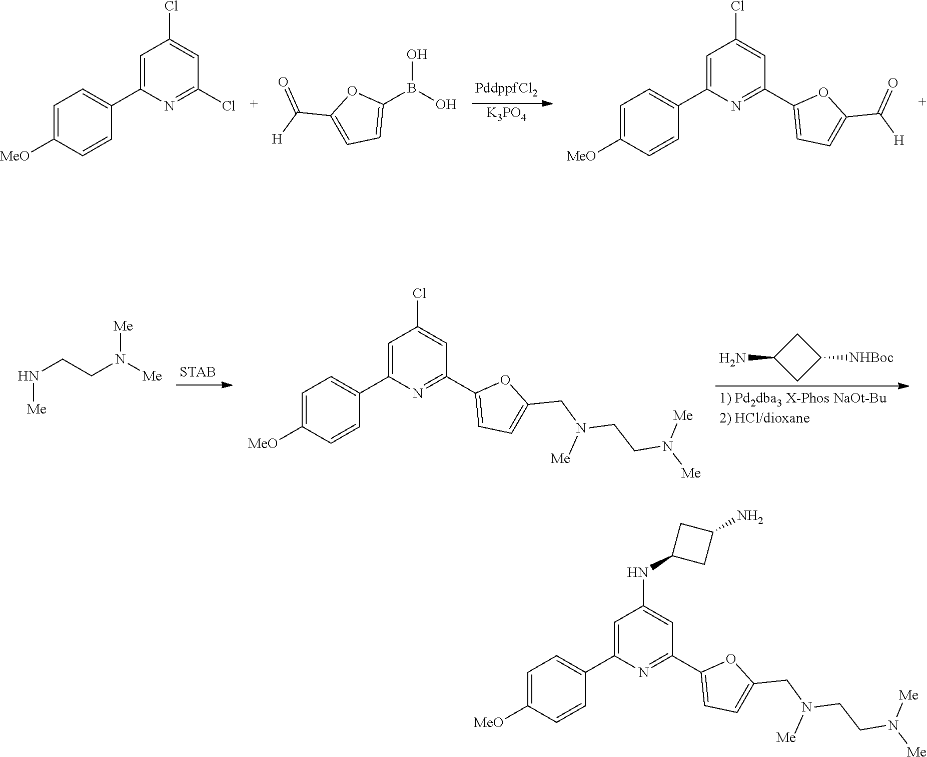

Example 96. 5-(4-chloro-6-(4-methoxyphenyl)pyridin-2-yl)furan-2-carbaldehyde. 2,4-dichloro-6-(4-methoxyphenyl)pyridine (1.0 g, 4.0 mmol), 5-formylfuran-2-yl-2-boronic acid (664 mg, 4.8 mmol), and tripotassium phosphate (1.79 g, 8.0 mmol) were dissolved in dioxane/water (20/4 mL). The mixture was degassed by gentle flow of nitrogen via canula, followed by the addition of PddpppfCl2 (30 mg, 0.04 mmol). The reaction mixture was heated to 80° C. overnight. The reaction was diluted with water and the aqueous layer extracted with ethyl acetate two times, the combined organic layers were washed with brine and dried over sodium sulfate and evacuated onto silica gel. Purified via column chromatography 40 g SiO₂ eluting with EtOAc/DCM (0-10%) to provide 0.480 g (31%) of 5-(4-chloro-6-(4-methoxyphenyl)pyridin-2-yl)furan-2-carbaldehyde as a pale solid. MS m z 314.45 (M+H).

N1-((5-(4-chloro-6-(4-methoxyphenyl)pyridin-2-yl)furan-2-yl)methyl)-N1,N2,N2-trimethylethane-1,2-diamine. To a solution of 5-(4-chloro-6-(4-methoxyphenyl)pyridin-2-yl)furan-2-carbaldehyde (100 mg, 0.32 mmol) and N1,N1,N2-trimethylethane-1,2-diamine (36 mg, 0.35 mmol) dissolved in dichloroethane (1.5 mL), was added sodium triacetoxyborohydride (102 mg, 0.48 mmol). The reaction mixture was stirred overnight at room temperature. The solvent was removed in vacuo and the crude product was purified on silica gel (12 g) eluting with a gradient of DCM/MeOH/NH₃ (90/9/1). Recovered 80 mg (63%) of N1-((5-(4-chloro-6-(4-methoxyphenyl)pyridin-2-yl)furan-2-yl)methyl)-N1,N2,N2-trimethylethane-1,2-diamine as clear oil. MS m z 400.44 (M+H).

N1-{2-[5-({[2-(dimethylamino)ethyl](methyl)amino}methyl)furan-2-yl]-6-(4-methoxyphenyl)pyridin-4-yl}cyclobutane-1,3-diamine. To a solution of N1-((5-(4-chloro-6-(4-methoxyphenyl)pyridin-2-yl)furan-2-yl)methyl)-N1,N2,N2-trimethylethane-1,2-diamine (80 mg, 0.20 mmol) in t-butanol was added tert-butyl (1r,3r)-3-aminocyclobutylcarbamate (45 mg, 0.22 mmol), the solutions was degassed via canula for 10 minutes, and hereto was added Pd₂dba₃ (18 mg, 0.020 mmol), X-Phos (28 mg, 0.060 mmol) and sodium-tert-butoxide (57 mg, 0.60 mmol), the reaction vessel was briefly flushed with nitrogen, sealed and heated to 90° C. for 2 hours. The crude cooled reaction mixture was filtered through a plug of celite washing with ethyl acetate, concentrated in vacuo and purified on silica gel eluting with a gradient of methanol in dichloromethane. Recovered material was deprotected and converted to the HCl salt with 1 mL of 4N HCl in dioxane and stirred for one hour. 20 mg (17%) of N1-{2-[5-({[2-(dimethylamino)ethyl](methyl)amino}methyl)furan-2-yl]-6-(4-methoxyphenyl)pyridin-4-yl}cyclobutane-1,3-diamine was produced as a pale yellow solid. MS m z 450.52 (M+H). ¹H NMR (300 MHz, cd₃₀d) δ=7.86 (d, 1H), 7.78 (d, 1H), 7.68-7.46 (m, 1H), 7.45-7.28 (m, 1H), 7.15 (d, 2H), 7.11-7.06 (m, 1H), 7.00-6.72 (m, 1H), 4.00 (br. s., 1H), 3.95-3.86 (m, 3H), 3.85-3.69 (m, 4H), 3.32-3.30 (m, 2H), 3.30-3.30 (m, 1H), 3.06-3.00 (m, 6H), 3.00-2.95 (m, 3H), 2.93-2.52 (m, 4H).

Throughout this application, various publications are referenced by author name and date, or by patent number or patent publication number. The disclosures of these publications are hereby incorporated in their entireties by reference into this application in order to more fully describe the state of the art as known to those skilled therein as of the date of the invention described and claimed herein. However, the citation of a reference herein should not be construed as an acknowledgement that such reference is prior art to the present invention.

Those skilled in the art will recognize, or be able to ascertain using no more than routine experimentation, numerous equivalents to the specific procedures described herein. Such equivalents are considered to be within the scope of this invention and are covered by the following claims. For example, pharmaceutically acceptable salts other than those specifically disclosed in the description and Examples herein can be employed. Furthermore, it is intended that specific items within lists of items, or subset groups of items within larger groups of items, can be combined with other specific items, subset groups of items or larger groups of items whether or not there is a specific disclosure herein identifying such a combination.

What is claimed is:

1. A compound having formula (I): 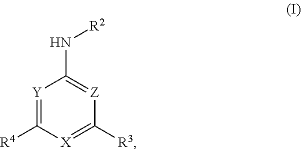
an enantiomer, a diastereomer, a hydrate, a solvate, a pharmaceutically acceptable salt, an isotopic analog, a prodrug or a complex thereof,
wherein, in formula (I):
X, Y, and Z are each independently N or CH;
R² is an amino(C₃-C₇)cycloalkyl group or a (C₁-C₅alkyl)₂amino(C₃-C₇)cycloalkyl group, wherein the (C₁-C₅alkyl) attached to “amino” are optionally connected to form a ring;
R³ is a substituted or unsubstituted phenyl group; and
R⁴ is an amino(C₃-C₇)cycloalkyl group, a (C₁-C₅alkyl)₂amino(C₃-C₇)cycloalkyl group wherein the (C₁-C₅alkyl) are optionally connected to form a ring, a heterocyclyl(C₂-C₅)alkyloxy group, or a heteroaryl(C₂-C₅)alkyloxy group, wherein “heterocyclyl” is a substituted or unsubstituted nitrogen-containing non-aromatic mono-heterocycle and wherein “heteroaryl” is a substituted or substituted nitrogen-containing aromatic mono-heterocycle.

2. The compound of claim 1, wherein X is CH, Y is CH, and Z is CH.

3. The compound of claim 1, wherein X is N, Y is CH, and Z is CH.

4. The compound of claim 1, wherein X is CH, Y is N, and Z is CH.

5. The compound of claim 1, wherein X is CH, Y is CH, and Z is N.

6. The compound of claim 1, wherein X is N, Y is N, and Z is CH.

7. The compound of claim 1, wherein X is CH, Y is N, and Z is N.

8. The compound of claim 1, wherein X is N, Y is CH, and Z is N.

9. The compound of claim 1, wherein X is N, Y is N, and Z is N.

10. The compound of claim 1, wherein R² is an amino(C₃-C₇)cycloalkyl group selected from an aminocyclopropyl group, an aminocyclobutyl group, an aminocyclopentyl group, an aminocyclohexyl group, or an aminocycloheptyl group, each of which has either cis-configuration or trans-configuration.

11. The compound of claim 1, wherein R² is a (C₁-C₅alkyl)₂amino(C₃-C₇)cycloalkyl group selected from a (C₁-C₅alkyl)₂aminocyclopropyl group, a (C₁-C₅alkyl)₂aminocyclobutyl group, a (C₁-C₅alkyl)₂aminocyclopentyl group, a (C₁-C₅alkyl)₂aminocyclohexyl group, or a (C₁-C₅alkyl)₂aminocycloheptyl group, each of which has either cis-configuration or trans-configuration.

12. The compound of claim 1, wherein R³ is a substituted or unsubstituted phenyl group.

13. The compound of claim 12, wherein R³ is a phenyl group, a chlorophenyl group, or a methoxyphenyl group.

14. The compound of claim 1, wherein R⁴ is a (C₀-C₅alkyl)₂amino(C₂-C₅)alkyloxy group selected from a (C₁-C₅alkyl)₂aminoethyloxy group, a (C₁-C₅alkyl)₂aminopropyloxy group, a (C₁-C₅alkyl)₂aminobutyloxy group, and a (C₁-C₅alkyl)₂aminopentyloxy group, wherein two “(C₁—C₅alkyl)₂ are optionally connected to form a ring.

15. The compound of claim 14, wherein the ring is a substituted or unsubstituted azetidine, a substituted or unsubstituted pyrrolidine, or a substituted or unsubstituted piperidine.

16. The compound of claim 1, wherein R⁴ is a (C₀-C₅alkyl)₂amino(C₂-C₅)alkyl group selected from a (C₁-C₅alkyl)₂aminoethyl group, a (C₁-C₅alkyl)₂aminopropyl group, a (C₁-C₅alkyl)₂aminobutyl group, and a (C₁-C₅alkyl)₂aminopentyl group, wherein two “(C₁-C₅alkyl)₂ are optionally connected to form a ring.

17. The compound of claim 16, wherein the ring is a substituted or unsubstituted azetidine, a substituted or unsubstituted pyrrolidine, or a substituted or unsubstituted piperidine.

18. The compound of claim 1, wherein R⁴ is a heterocyclyl(C₂-C₅)alkyloxy group or a heteroaryl(C₂-C₅)alkyloxy group, wherein “heterocyclyl” is a substituted or unsubstituted nitrogen-containing non-aromatic mono-heterocycle and wherein “heteroaryl” is a substituted or substituted nitrogen-containing aromatic mono-heterocycle.

19. The compound of claim 18, wherein “heterocyclyl” and the “heteroaryl” are connected to the “(C₂-C₅)alkyloxy” through a nitrogen atom.
The compound of claim 1, wherein

20. The compound of claim 1, wherein R⁴ is a substituted or unsubstituted pyrrolidinoethyloxy group or substituted or unsubstituted pyrrolidinopropyloxy group.

21. A method for treating or preventing a disease that involves TDP-43, the method comprising administering to a subject an effective amount of at least one compound having formula (I): 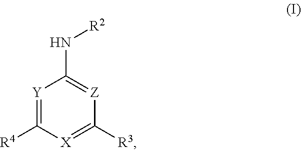
an enantiomer, a diastereomer, a hydrate, a solvate, a pharmaceutically acceptable salt, an isotopic analog, a prodrug or a complex thereof,
wherein, in formula (I):
X, Y, and Z are each independently N or CH;
R² is a substituted or unsubstituted C₁-C₂₀ linear, branched, or cyclic organic group including at least one nitrogen; and
R³ and R⁴ are each independently hydrogen, halogen, a hydroxyl group, a cyano group, a nitro group, a substituted or unsubstituted C₀-C₁₀ amino group, a substituted or unsubstituted C₁-C₁₀ alkyl group, a substituted or unsubstituted C₂-C₁₀ alkenyl group, a substituted or unsubstituted C₂-C₁₀ alkynyl group, and a substituted or unsubstituted C₁-C₁₀ alkoxy group, a substituted or unsubstituted C₃-C₁₀ cycloalkyl group, a substituted or unsubstituted C₂-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₂₀ aryl group, a substituted or unsubstituted C₆-C₂₀ aryloxy group, a substituted or unsubstituted C₆-C₂₀ arylthio group, a substituted or unsubstituted C₂-C₂₀ heteroaryl group, a substituted or unsubstituted C₂-C₂₀ heteroaryloxy group, or a substituted or unsubstituted C₂-C₂₀ heteroarylthio group.

22. A compound having formula (II): 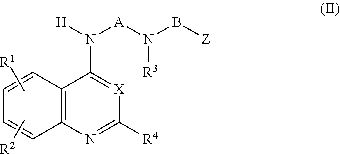
an enantiomer, a diastereomer, a hydrate, a solvate, a pharmaceutically acceptable salt, an isotopic analog, a prodrug or a complex thereof,
wherein, in formula (II):
X is N or CH;
R¹ and R² are the same or different and are each independently H, halogen, C₁-C₄ alkyl, C₁-C₄ alkoxy, CF₃, or CF₃O, wherein C₁-C₄ alkyl and C₁-C₄ alkoxy are optionally substituted with at least one fluorine;
R³ is H, C₁-C₇ alkyl, C₄-C₇ carbocyclic, aryl, heteroaryl, heterocyclic, heterocyclic alkyl, wherein R³ is optionally taken together with either A or B to form a ring consisting of 4-7 ring members;
R⁴=phenyl or phenyl substituted with C₁-C₄ alkyl, C₁-C₄ alkoxy, CF₃, CF₃O, halogen, amino, or sulfonamide;
A=(CH₂)ₙ, wherein n is 2-4, wherein A and R³ are optionally taken together to form a ring consisting of 4-7 ring members;
B=(CH₂)ₙ, wherein n is 2-6, wherein B and R³ are optionally taken together to form a ring consisting of 4-7 members, and wherein B is optionally substituted with one or more substituents selected from C₁-C₄ alkyl, hydroxy, C₁-C₄ alkoxy, CF₃, and CF₃O; and
Z is OH, C₁-C₄ alkoxy, OCF₃, or N with one or more of H, C₁-C₄ alkyl, heteroaryl, substituted heteroaryl, C₁-C₄ sulfonamido, substituted amido.

23. The compound of claim 22, wherein
X=CH;
R¹ and R² are each H,
R³ is H or Me,
R⁴ is 4-methoxyphenyl,
A is (CH₂)₃,
B is (CH₂)ₙ, wherein n is 2-4 and Z=N(R⁶)₂, wherein each R⁶ is independently H, C₁-C₄ alkyl, heteroaryl, substituted heteroaryl, C₁-C₄ sulfonamido, or substituted amido.

24. A method for treating or preventing a disease that involves TDP-43, the method comprising administering to a subject an effective amount of at least one compound having formula (II): 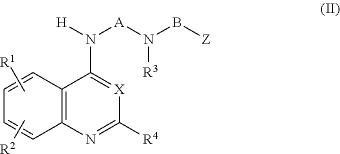
an enantiomer, a diastereomer, a hydrate, a solvate, a pharmaceutically acceptable salt, an isotopic analog, a prodrug or a complex thereof,
wherein, in formula (II):
X is N or CH;
R¹ and R² are the same or different and are each independently H, halogen, C₁-C₄ alkyl, C₁-C₄ alkoxy, CF₃, or CF₃O, wherein C₁-C₄ alkyl and C₁-C₄ alkoxy are optionally substituted with at least one fluorine;
R³ is H, C₁-C₇ alkyl, C₄-C₇ carbocyclic, aryl, heteroaryl, heterocyclic, heterocyclic alkyl, wherein R³ is optionally taken together with either A or B to form a ring consisting of 4-7 ring members;
R⁴=phenyl or phenyl substituted with C₁-C₄ alkyl, C₁-C₄ alkoxy, CF₃, CF₃O, halogen, amino, or sulfonamide;
A=(CH₂)ₙ, wherein n is 2-4, wherein A and R³ are optionally taken together to form a ring consisting of 4-7 ring members;
B=(CH₂)ₙ, wherein n is 2-6, wherein B and R³ are optionally taken together to form a ring consisting of 4-7 members, and wherein B is optionally substituted with one or more substituents selected from C₁-C₄ alkyl, hydroxy, C₁-C₄ alkoxy, CF₃, and CF₃O; and
Z is OH, C₁-C₄ alkoxy, OCF₃, or N with one or more of H, C₁-C₄ alkyl, heteroaryl, substituted heteroaryl, C₁-C₄ sulfonamido, substituted amido.

25. The method of claim 24, wherein
X=CH;
R¹ and R² are each H,
R³ is H or Me,
R⁴ is 4-methoxyphenyl,
A is (CH₂)₃,
B is (CH₂)ₙ, wherein n is 2-4 and Z=N(R⁶)₂, wherein each R⁶ is independently H, C₁-C₄ alkyl, heteroaryl, substituted heteroaryl, C₁-C₄ sulfonamido, or substituted amido.

26. A compound having formulae (IIIa) or (IIIb): 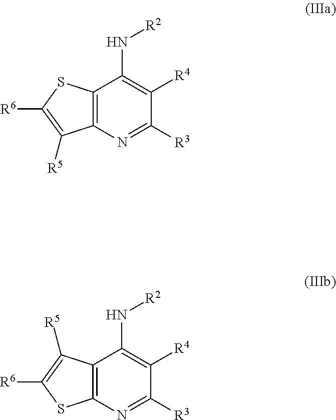
an enantiomer, a diastereomer, a hydrate, a solvate, a pharmaceutically acceptable salt, an isotopic analog, a prodrug or a complex thereof,
wherein, in formulae (IIIa) and (IIIb):
R² is a substituted or unsubstituted C₁-C₂₀ linear, branched, or cyclic organic group including at least one nitrogen; and
R³, R⁴, R⁵, and R⁶ are each independently hydrogen, halogen, a hydroxyl group, a cyano group, a nitro group, a substituted or unsubstituted C₀-C₁₀ amino group, a substituted or unsubstituted C₁-C₁₀ alkyl group, a substituted or unsubstituted C₂-C₁₀ alkenyl group, a substituted or unsubstituted C₂-C₁₀ alkynyl group, and a substituted or unsubstituted C₁-C₁₀ alkoxy group, a substituted or unsubstituted C₃-C₁₀ cycloalkyl group, a substituted or unsubstituted C₂-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₂₀ aryl group, a substituted or unsubstituted C₆-C₂₀ aryloxy group, a substituted or unsubstituted C₆-C₂₀ arylthio group, a substituted or unsubstituted C₂-C₂₀ heteroaryl group, a substituted or unsubstituted C₂-C₂₀ heteroaryloxy group, or a substituted or unsubstituted C₂-C₂₀ heteroarylthio group.

27. A method for treating or preventing a disease that involves TDP-43, the method comprising administering to a subject an effective amount of at least one compound having formulae (IIIa) or (IIIb): 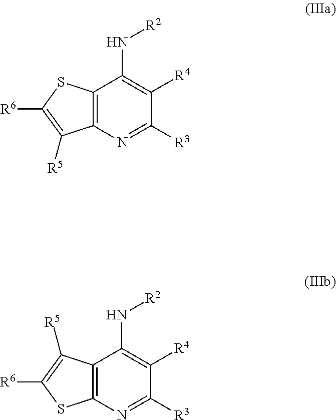
an enantiomer, a diastereomer, a hydrate, a solvate, a pharmaceutically acceptable salt, an isotopic analog, a prodrug or a complex thereof,
wherein, in formulae (IIIa) and (IIIb):
R² is a substituted or unsubstituted C₁-C₂₀ linear, branched, or cyclic organic group including at least one nitrogen; and
R³, R⁴, R⁵, and R⁶ are each independently hydrogen, halogen, a hydroxyl group, a cyano group, a nitro group, a substituted or unsubstituted C₀-C₁₀ amino group, a substituted or unsubstituted C₁-C₁₀ alkyl group, a substituted or unsubstituted C₂-C₁₀ alkenyl group, a substituted or unsubstituted C₂-C₁₀ alkynyl group, and a substituted or unsubstituted C₁-C₁₀ alkoxy group, a substituted or unsubstituted C₃-C₁₀ cycloalkyl group, a substituted or unsubstituted C₂-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₂₀ aryl group, a substituted or unsubstituted C₆-C₂₀ aryloxy group, a substituted or unsubstituted C₆-C₂₀ arylthio group, a substituted or unsubstituted C₂-C₂₀ heteroaryl group, a substituted or unsubstituted C₂-C₂₀ heteroaryloxy group, or a substituted or unsubstituted C₂-C₂₀ heteroarylthio group.

28. A compound having formula (IV): 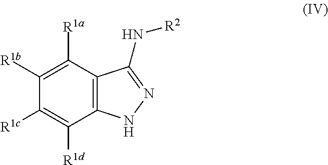
an enantiomer, a diastereomer, a hydrate, a solvate, a pharmaceutically acceptable salt, an isotopic analog, a prodrug or a complex thereof,
wherein, in formula (IV):
R² is a substituted or unsubstituted C₁-C₂₀ linear, branched, or cyclic organic group including at least one nitrogen; and
R^{1a}, R^{1l}, R^{1c}, and R^{1d} are each independently hydrogen, halogen, a hydroxyl group, a cyano group, a nitro group, a substituted or unsubstituted C₀-C₁₀ amino group, a substituted or unsubstituted C₁-C₁₀ alkyl group, a substituted or unsubstituted C₂-C₁₀ alkenyl group, a substituted or unsubstituted C₂-C₁₀ alkynyl group, and a substituted or unsubstituted C₁-C₁₀ alkoxy group, a substituted or unsubstituted C₃-C₁₀ cycloalkyl group, a substituted or unsubstituted C₂-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₂₀ aryl group, a substituted or unsubstituted C₆-C₂₀ aryloxy group, a substituted or unsubstituted C₆-C₂₀ arylthio group, a substituted or unsubstituted C₂-C₂₀ heteroaryl group, a substituted or unsubstituted C₂-C₂₀ heteroaryloxy group, or a substituted or unsubstituted C₂-C₂₀ heteroarylthio group.

29. A method for treating or preventing a disease that involves TDP-43, the method comprising administering to a subject an effective amount of at least one compound having formula (IV): 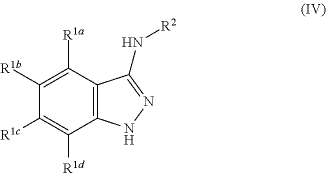
an enantiomer, a diastereomer, a hydrate, a solvate, a pharmaceutically acceptable salt, an isotopic analog, a prodrug or a complex thereof,
wherein, in formula (IV):
R² is a substituted or unsubstituted C₁-C₂₀ linear, branched, or cyclic organic group including at least one nitrogen; and
R^{1a}, R^{1l}, R^{1c}, and R^{1d} are each independently hydrogen, halogen, a hydroxyl group, a cyano group, a nitro group, a substituted or unsubstituted C₀-C₁₀ amino group, a substituted or unsubstituted C₁-C₁₀ alkyl group, a substituted or unsubstituted C₂-C₁₀ alkenyl group, a substituted or unsubstituted C₂-C₁₀ alkynyl group, and a substituted or unsubstituted C₁-C₁₀ alkoxy group, a substituted or unsubstituted C₃-C₁₀ cycloalkyl group, a substituted or unsubstituted C₂-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₂₀ aryl group, a substituted or unsubstituted C₆-C₂₀ aryloxy group, a substituted or unsubstituted C₆-C₂₀ arylthio group, a substituted or unsubstituted C₂-C₂₀ heteroaryl group, a substituted or unsubstituted C₂-C₂₀ heteroaryloxy group, or a substituted or unsubstituted C₂-C₂₀ heteroarylthio group.

30. A compound having formula (V): 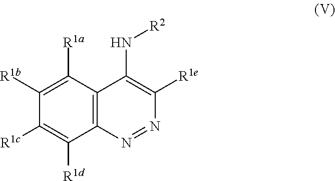
an enantiomer, a diastereomer, a hydrate, a solvate, a pharmaceutically acceptable salt, an isotopic analog, a prodrug or a complex thereof,
wherein, in formula (V):
R² is a substituted or unsubstituted C₁-C₂₀ linear, branched, or cyclic organic group including at least one nitrogen; and
R^{1a}, R^{1b}, R^{ic}, R^{1d}, and R^{1c} are each independently hydrogen, halogen, a hydroxyl group, a cyano group, a nitro group, a substituted or unsubstituted C₀-C₁₀ amino group, a substituted or unsubstituted C₁-C₁₁ alkyl group, a substituted or unsubstituted C₂-C₁₁ alkenyl group, a substituted or unsubstituted C₂-C₁₀ alkynyl group, and a substituted or unsubstituted C₁-C₁₀ alkoxy group, a substituted or unsubstituted C₃-C₁₀ cycloalkyl group, a substituted or unsubstituted C₂-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₂₀ aryl group, a substituted or unsubstituted C₆-C₂₀ aryloxy group, a substituted or unsubstituted C₆-C₂₀ arylthio group, a substituted or unsubstituted C₂-C₂₀ heteroaryl group, a substituted or unsubstituted C₂-C₂₀ heteroaryloxy group, or a substituted or unsubstituted C₂-C₂₀ heteroarylthio group.

31. A method for treating or preventing a disease that involves TDP-43, the method comprising administering to a subject an effective amount of at least one compound having formula (V): 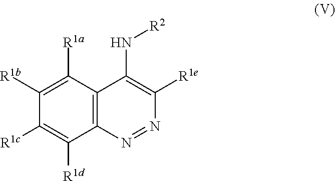
an enantiomer, a diastereomer, a hydrate, a solvate, a pharmaceutically acceptable salt, an isotopic analog, a prodrug or a complex thereof,
wherein, in formula (V):
R² is a substituted or unsubstituted C₁-C₂₀ linear, branched, or cyclic organic group including at least one nitrogen; and
R^{1a}, R^{1b}, R^{1c}, R^{1d}, and R^{1e} are each independently hydrogen, halogen, a hydroxyl group, a cyano group, a nitro group, a substituted or unsubstituted C₀-C₁₀ amino group, a substituted or unsubstituted C₁-C₁₀ alkyl group, a substituted or unsubstituted C₂-C₁₀ alkenyl group, a substituted or unsubstituted C₂-C₁₀ alkynyl group, and a substituted or unsubstituted C₁-C₁₀ alkoxy group, a substituted or unsubstituted C₃-C₁₀ cycloalkyl group, a substituted or unsubstituted C₂-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₂₀ aryl group, a substituted or unsubstituted C₆-C₂₀ aryloxy group, a substituted or unsubstituted C₆-C₂₀ arylthio group, a substituted or unsubstituted C₂-C₂₀ heteroaryl group, a substituted or unsubstituted C₂-C₂₀ heteroaryloxy group, or a substituted or unsubstituted C₂-C₂₀ heteroarylthio group.

32. A pharmaceutical composition comprising the compound of any one of the preceding claims and at least one excipient.

33. The method of any one of the preceding claims, wherein the at least one compound is administered in a composition further comprising at least one excipient.

34. The method of any one of the preceding claims, wherein the diseases that involves TDP-43 is selected from the group consisting of amyotrophic lateral sclerosis (ALS), frontotemporal lobar degeneration (FTLD), chronic traumatic encephalopathy (CTE), hippocampal sclerosis of aging (CARTS), inclusion body myositis (IBM), Alzheimer's disease (AD), and Alzheimer's disease (AD) related disorders.

35. A method for treating or preventing a disease associated with TDP-43 proteinopathies, the method comprising administering to a subject an effective amount of at least one compound recited in any one of the preceding claims.

36. The method of claim 35, wherein the at least one compound is administered in a composition further comprising at least one excipient.

37. A method for treating or preventing diseases that involve excess amounts of TDP-43 in the cytosol, the method comprising administering to a subject an effective amount of at least one compound recited in any one of the preceding claims.

38. The method of claim 37, wherein the at least one compound is administered in a composition further comprising at least one excipient.

39. A method of use of the TDP-43 binders of the present invention as positron emission tomography (PET) imaging agents, wherein the method comprises administering to a subject an effective amount of an isotopically labeled compound recited in any one of the preceding claims.

40. A method of use of the TDP-43 binders of the present invention as single-photon emission computed tomography (SPECT) imaging agents, wherein the method comprises administering to a subject an effective amount of an isotopically labeled compound recited in any one of the preceding claims.

41. The method of any one of the preceding claims, wherein the compound is administered in a combination with at least one other agents known to treat amyotrophic lateral sclerosis (ALS), frontotemporal lobar degeneration (FTLD), chronic traumatic encephalopathy (CTE), hippocampal sclerosis of aging (CARTS), inclusion body myositis (IBM), Alzheimer's disease (AD), and Alzheimer's disease (AD) related disorders.

42. The method of claim 41, wherein the at least one other agent is riluzole, troriluzole, or edavarone.

43. A compound selected from Compounds 1-96 listed in Table 1 of the present application.

44. A method for treating or preventing a disease that involves TDP-43, the method comprising administering to a subject an effective amount of at least one compound selected from Compounds 1-96 listed in Table 1 of the present application, an enantiomer, a diastereomer, a hydrate, a solvate, a pharmaceutically acceptable salt, an isotopic analog, a prodrug or a complex thereof.
